SEQUENCE LISTING (130 sequences)

SEQ ID NO: 1 - protein, 14 aa, synthetic construct
WVSYGNNRNWFFDF

SEQ ID NO: 2 - protein, 8 aa, synthetic construct
IRATGFDS

SEQ ID NO: 3 - protein, 7 aa, synthetic construct
YGNSGAN

SEQ ID NO: 4 - protein, 11 aa, synthetic construct
FQYGDYRYFDV

SEQ ID NO: 5 - protein, 7 aa, synthetic construct
YGGGFAY

SEQ ID NO: 6 - protein, 4 aa, synthetic construct
NYFD

SEQ ID NO: 7 - protein, 11 aa, synthetic construct
GGYDGSREFAY

SEQ ID NO: 8 - protein, 11 aa, synthetic construct
FQYGDYRYFDV

SEQ ID NO: 9 - protein, 14 aa, synthetic construct
RGIYYGYDGYAMDY

SEQ ID NO: 10 - protein, 14 aa, synthetic construct
RGIYYGYEGYAMDY

SEQ ID NO: 11 - protein, 14 aa, synthetic construct
RGIYYGYSGYAMDY

SEQ ID NO: 12 - protein, 14 aa, synthetic construct
RGIYYGYAGYAMDY

SEQ ID NO: 13 - protein, 17 aa, synthetic construct
WINTYTGAPVYAADFKG

SEQ ID NO: 14 - protein, 18 aa, synthetic construct
SFNPNNGGLTNYNQKFKG

SEQ ID NO: 15 - protein, 17 aa, synthetic construct
YINPNNDNINYNEKFKG

SEQ ID NO: 16 - protein, 17 aa, synthetic construct
WINTYTGNPIYAADFKG

SEQ ID NO: 17 - protein, 17 aa, synthetic construct
YINPYSDVTNCNEKFKG

SEQ ID NO: 18 - protein, 17 aa, synthetic construct
YINPYSDVTNYNEKFKG

SEQ ID NO: 19 - protein, 17 aa, synthetic construct
WINTYSGVPTYADDFKG

SEQ ID NO: 20 - protein, 16 aa, synthetic construct
WINTYTGNPIYAADFK

SEQ ID NO: 21 - protein, 17 aa, synthetic construct
TISSGSIYIYYTDGVKG

SEQ ID NO: 22 - protein, 17 aa, synthetic construct
TISSGSIYIYYTDSVKG

SEQ ID NO: 23 - protein, 10 aa, synthetic construct
GYSFRDYGMN

SEQ ID NO: 24 - protein, 10 aa, synthetic construct
GYTFTDYTMH

SEQ ID NO: 25 - protein, 10 aa, synthetic construct
GYTFTSYVMH

SEQ ID NO: 26 - protein, 10 aa, synthetic construct
GYTFTNYGVK

SEQ ID NO: 27 - protein, 10 aa, synthetic construct
GYTVTSYVMH

SEQ ID NO: 28 - protein, 10 aa, synthetic construct
GYTFSSYVMH

SEQ ID NO: 29 - protein, 10 aa, synthetic construct
GFTFTNYPMH

SEQ ID NO: 30 - protein, 10 aa, synthetic construct
GYTFTNYGVK

SEQ ID NO: 31 - protein, 10 aa, synthetic construct
GFTFSSFAMS

SEQ ID NO: 32 - protein, 9 aa, synthetic construct
FQGSHGPWT

SEQ ID NO: 33 - protein, 9 aa, synthetic construct
KQSYNPPWT

SEQ ID NO: 34 - protein, 9 aa, synthetic construct
QHHYGTPWT

SEQ ID NO: 35 - protein, 9 aa, synthetic construct
QQWSSNPLT

SEQ ID NO: 36 - protein, 9 aa, synthetic construct
QHHYDTPYT

SEQ ID NO: 37 - protein, 9 aa, synthetic construct
QHHYNIPFT

SEQ ID NO: 38 - protein, 8 aa, synthetic construct
QHIRELTT

SEQ ID NO: 39 - protein, 9 aa, synthetic construct
QQWSSNPLT

SEQ ID NO: 40 - protein, 9 aa, synthetic construct
SQSTHIPLA

SEQ ID NO: 41 - protein, 7 aa, synthetic construct
KVSNRLS

SEQ ID NO: 42 - protein, 7 aa, synthetic construct
WASTRES

SEQ ID NO: 43 - protein, 7 aa, synthetic construct
HTKTLAA

SEQ ID NO: 44 - protein, 7 aa, synthetic construct
GTANLAS

SEQ ID NO: 45 - protein, 7 aa, synthetic construct
NARTLAG

SEQ ID NO: 46 - protein, 7 aa, synthetic construct
NAKTLAE

SEQ ID NO: 47 - protein, 7 aa, synthetic construct
LVSNLES

SEQ ID NO: 48 - protein, 7 aa, synthetic construct
GTANLAS

SEQ ID NO: 49 - protein, 7 aa, synthetic construct
KVSNRFS

SEQ ID NO: 50 - protein, 16 aa, synthetic construct
RSSQTLVHSNGNTYLE

SEQ ID NO: 51 - protein, 17 aa, synthetic construct
KSSQSLLNSRTRKNYLA

SEQ ID NO: 52 - protein, 11 aa, synthetic construct
RTSKNIFNFLA

SEQ ID NO: 53 - protein, 10 aa, synthetic construct
RANSGISFIN

SEQ ID NO: 54 - protein, 11 aa, synthetic construct
RASKNIYTYLA

SEQ ID NO: 55 - protein, 11 aa, synthetic construct
RASKNVYSYLA

SEQ ID NO: 56 - protein, 15 aa, synthetic construct
RASKSVSTSGYSYMH

SEQ ID NO: 57 - protein, 10 aa, synthetic construct
RANSGISFIN

SEQ ID NO: 58 - protein, 10 aa, synthetic construct
RAQSGISFIN

SEQ ID NO: 59 - protein, 16 aa, synthetic construct
RSSQSLVHSNGNTYLH

SEQ ID NO: 60 - protein, 16 aa, synthetic construct
RSSQSLVHSSGNTYLH

SEQ ID NO: 61 - protein, 16 aa, synthetic construct
RSSQSLVHSTGNTYLH

SEQ ID NO: 62 - protein, 16 aa, synthetic construct
RSSQSLVHSQGNTYLH

SEQ ID NO: 63 - protein, 123 aa, synthetic construct
QIQLVQSGPELKKPGETVKISCKASGYSFRDYGMNWVKQAPGRTFKWMGWINTYTGAPVYAADFKGRFAFSLDTSASAAFLQINNLKNEDTATYFCARWVSYGNNRNWFFDFWGAGTTVTVSS

SEQ ID NO: 64 - protein, 118 aa, synthetic construct
EVQLQQSGPELVKPGASVKISCKTSGYTFTDYTMHWVRQSHGKSLEWIGSFNPNNGGLTNYNQKFKGKATLTVDKSSSTVYMDLRSLTSEDSAVYYCTRIRATGFDSWGQGTTLTVSS

SEQ ID NO: 65 - protein, 116 aa, synthetic construct
EVQVQQSGPELVKPGASVKMSCKASGYTFTSYVMHWVKQKPGQGLEWIGYINPNNDNINYNEKFKGKASLTSDKSSNTVYMELSSLTSEDSAVYYCAGYGNSGANWGQGTLVTVSA

SEQ ID NO: 66 - protein, 120 aa, synthetic construct
QIQLVQSGPELKKPGETVKISCKASGYTFTNYGVKWVKQAPGKDLKWMGWINTYTGNPIYAADFKGRFAFSLETSASTAFLQINNLKNEDTATYFCVRFQYGDYRYFDVWGAGTTVTVSS

SEQ ID NO: 67 - protein, 116 aa, synthetic construct
EVQLQQSGPELVKPGASVKLSCKASGYTVTSYVMHWVKQKPGQGLEWIGYINPYSDVTNCNEKFKGKATLTSDKTSSTAYMELSSLTSEDSAVYYCSSYGGGFAYWGQGTLVTVSA

SEQ ID NO: 68 - protein, 113 aa, synthetic construct
EVQLQQSGPELVKPGASVKMSCKASGYTFSSYVMHWVKQKPGQGLEWIGYINPYSDVTNYNEKFKGKATLTSDRSSNTAYMELSSLTSEDSAVYYCARNYFDWGRGTLVTVSA

SEQ ID NO: 69 - protein, 119 aa, synthetic construct
QIQLVQSGPELKKPGETVKISCKASGFTFTNYPMHWVKQAPGKGLKWMGWINTYSGVPTYADDFKGRFAFSLETSASTAYLQINNLKNEDMATYFCARGGYDGSREFAYWGQGTLVTVS

SEQ ID NO: 70 - protein, 450 aa, synthetic construct
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 450 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 453 aa, synthetic construct
EVQLVESGGGLVKPGGSLRLSCAASGFTFSSFAMSWVRQAPGKGLEWVSTISSGSIYIYYTDGVKGRFTISRDNAKNSLYLQMNSLRAEDTAVYYCARRGIYYGYDGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 453 aa, synthetic construct
EVQLVESGGGLVKPGGSLRLSCAASGFTFSSFAMSWVRQAPGKGLEWVSTISSGSIYIYYTDSVKGRFTISRDNAKNSLYLQMNSLRAEDTAVYYCARRGIYYGYEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 112 aa, synthetic construct
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 75 - protein, 112 aa, synthetic construct
DVLMTQTPLSLPVRLGDQASISCRSSQTLVHSNGNTYLEWYLQKPGQSPKLLIYKVSNRLSGVPDRFSGSGSGTDFTLKISRVETEDLGVYYCFQGSHGPWTFGGGTKLEIK

SEQ ID NO: 76 - protein, 113 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISNVQAEDLTVYYCKQSYNPPWTFGGGTKLEIK

SEQ ID NO: 77 - protein, 107 aa, synthetic construct
DIQMTQSPASLSASVGETVTITCRTSKNIFNFLAWYHQKQGRSPRLLVSHTKTLAAGVPSRFSGSGSGTQFSLKINSLQPEDFGIYYCQHHYGTPWTFGGGTKLEIK

SEQ ID NO: 78 - protein, 106 aa, synthetic construct
QIILSQSPAILSASPGEKVTMTCRANSGISFINWYQQKPGSSPKPWIYGTANLASGVPARFGGSGSGTSYSLTISRVEAEDAATYYCQQWSSNPLTFGAGTKLELR

SEQ ID NO: 79 - protein, 107 aa, synthetic construct
DIQMTQSPASLSASVGEPVTITCRASKNIYTYLAWYHQKQGKSPQFLVYNARTLAGGVPSRLSGSGSVTQFSLNINTLHREDLGTYFCQHHYDTPYTFGGGTNLEIK

SEQ ID NO: 80 - protein, 107 aa, synthetic construct
DIQMTQSPASLSASVGETVTITCRASKNVYSYLAWFQQKQGKSPQLLVYNAKTLAEGVPSRFSGGGSGTQFSLKINSLQPADFGSYYCQHHYNIPFTFGSGTKLEIK

SEQ ID NO: 81 - protein, 110 aa, synthetic construct
DIVLTQSPASLAASLGQRATTSYRASKSVSTSGYSYMHWNQQKPGQPPRLLIYLVSNLESGVPARFSGSGSGTDFTLNIHPVEEEDAATYYCQHIRELTTFGGGTKLEIK

SEQ ID NO: 82 - protein, 213 aa, synthetic construct
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 213 aa, synthetic construct
EIVLTQSPATLSLSPGERATLSCRANSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 213 aa, synthetic construct
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 219 aa, synthetic construct
AIVLTQSPGTLSLSPGERATLSCRSSQSLVHSNGNTYLHWYMQKPGQAPRVLIYKVSNRFSGIPDRFSGSGSGTDFTLTISRLEPDDFAIYYCSQSTHIPLAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 249 aa, synthetic construct
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVSGGGGSGGGGSGGGGSAVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 87 - protein, 250 aa, synthetic construct
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELKGGGGSGGGGSGGGGSEVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVSS

SEQ ID NO: 88 - protein, 122 aa, synthetic construct
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVS

SEQ ID NO: 89 - DNA, 20 bp, synthetic construct
  source 1..20: mol_type = other DNA
agtgcactcggaccatgtgg

SEQ ID NO: 90 - DNA, 20 bp, synthetic construct
  source 1..20: mol_type = other DNA
ggtgtagttccactggccga

SEQ ID NO: 91 - DNA, 20 bp, synthetic construct
  source 1..20: mol_type = other DNA
attagccgcgatgcacagga

SEQ ID NO: 92 - DNA, 369 bp, synthetic construct
  source 1..369: mol_type = other DNA
caaattcagttggttcaatccggccctgagctcaagaagcctggagagacagtgaagataagttgtaaggctagtggctattcatttcgagattatgggatgaattgggtcaagcaggccccagggcggaccttcaaatggatggggtggatcaatacttacactggcgcaccagtatatgcagctgattttaagggtcgctttgcattttcacttgatacttcagccagtgccgcttttttgcaaatcaacaatctcaaaaatgaagacactgctacatatttctgcgccaggtgggtgagctatggcaataacagaaattggttctttgacttttggggcgcaggcaccaccgtcactgtctcatca

SEQ ID NO: 93 - DNA, 354 bp, synthetic construct
  source 1..354: mol_type = other DNA
gaggtacaactgcaacagagtggacctgaacttgtcaaacctggagcaagtgtgaagattagctgtaaaaccagtggctacacatttaccgattatactatgcactgggtaagacagagccacggaaaatcactggagtggattggtagtttcaatcctaacaacggaggattgacaaattacaaccagaagttcaaagggaaagccaccttgacagttgataagtcctcaagtaccgtgtatatggatctgcgttctctcacaagtgaagatagcgcagtttactactgtacccgcatccgagccaccgggttcgattcatggggtcaggggacaacactgactgtttcttct

SEQ ID NO: 94 - DNA, 348 bp, synthetic construct
  source 1..348: mol_type = other DNA
gaagttcaagttcagcaaagcgggcctgagcttgtcaagccaggcgcatcagtcaaaatgagctgtaaggcttccgggtacaccttcaccagttatgtcatgcattgggtaaaacaaaagccaggacagggactcgagtggataggatacattaacccaaataacgacaacattaactacaacgagaaattcaagggcaaagcatcattgacttccgataaatcctctaacaccgtgtacatggagctgagttcattgaccagcgaggattctgccgtgtactactgtgcaggttatggcaactctggtgctaactgggggcaggggactctggtcacagtcagcgca

SEQ ID NO: 95 - DNA, 360 bp, synthetic construct
  source 1..360: mol_type = other DNA
caaatccaacttgtccagagcggtcccgagttgaagaagcctggcgaaaccgtgaaaatctcatgcaaggccagtggatatacatttacaaactatggcgtcaagtgggtgaaacaagccccaggtaaagacttgaaatggatgggatggatcaacacatacacagggaatcctatctatgcagccgactttaaaggcagatttgccttcagtttggagacatctgcctccaccgctttcctgcaaataaataacctgaaaaatgaagataccgctacatacttctgtgtacggttccagtacggagattaccgctatttcgatgtgtggggcgcaggtaccacagtaaccgtctcctca

SEQ ID NO: 96 - DNA, 348 bp, synthetic construct
  source 1..348: mol_type = other DNA
gaagtccagcttcagcaatccggcccagaactggtaaaaccaggcgcaagtgttaagttgagttgcaaagccagtggttataccgttacttcatacgtcatgcattgggtaaaacaaaagcccggccaagggcttgaatggatcggctacatcaacccttactctgacgtcaccaactgcaacgagaaattcaaagggaaagccacattgacctctgacaagacaagcagtaccgcctacatggagctttctagtttgacttctgaagactctgctgtctactactgtagcagctacggcggcggctttgcttactggggccagggtacattggtgactgtgagtgca

SEQ ID NO: 97 - DNA, 339 bp, synthetic construct
  source 1..339: mol_type = other DNA
gaggtacagcttcagcagagtggtccagaactcgtcaagcctggggcaagcgttaagatgagttgtaaagcatccggttacacattcagtagctatgttatgcactgggtcaaacagaagcctgggcaggggttggagtggatcggatatataaatccctattcagacgtaactaattataatgaaaagttcaaggggaaagcaaccttgacaagtgaccggtcatctaataccgcatacatggagctgagctcattgacaagtgaggactctgctgtgtattactgtgcccggaactacttcgactggggtaggggcacactggtaactgttagtgca

SEQ ID NO: 98 - DNA, 356 bp, synthetic construct
  source 1..356: mol_type = other DNA
cagatacaactcgtccagtcaggtccagagttgaagaaacccggagaaactgtgaagatatcctgtaaagccagcggctttactttcacaaactaccccatgcattgggtgaagcaggccccggaaaaggactcaaatggatgggatggatcaacacatacagtggggtgcctacttacgcagacgatttcaaaggaaggttcgcatttagcttggaaactagcgcatctacagcatatctccagattaacaatcttaaaaatgaggatatggcaacatacttctgcgctaggggaggttacgatgggagcagggagttcgcttattgggggcaagggactcttgtgactgtaagt

SEQ ID NO: 99 - DNA, 336 bp, synthetic construct
  source 1..336: mol_type = other DNA
gacgtacttatgacacaaactcccttgagcttgccagtacggcttggcgatcaagcttcaatttcatgtcgttcttctcaaacacttgtccactcaaatgggaatacatatttggaatggtatctccaaaagcccggccaatccccaaaattgttgatttacaaggtgtctaatcgactctcaggcgtccccgaccgattctccgggagcgggtccggtacagacttcaccttgaaaatctccagggtagaaactgaagacctcggagtctactattgtttccaggggtcacacggcccctggacatttggaggaggaactaagctcgagatcaaa

SEQ ID NO: 100 - DNA, 339 bp, synthetic construct
  source 1..339: mol_type = other DNA
gacatagttatgtcccagtctccatccagcttggctgtcagcgccggagagaaagtgactatgagttgtaaatcttcccagtccctgcttaactcacgtactcggaagaattatcttgcctggtatcaacaaaagccaggtcaaagtcctaagctccttatttactgggcctcaacacgggagtcaggtgtccccgatcgcttcacaggtagtgggagtggtactgacttcactctcaccatttcaaatgtccaagcagaagacttgactgtgtattactgtaagcagagttacaaccctccttggacctttggtggggggaccaaactggagatcaag

SEQ ID NO: 101 - DNA, 321 bp, synthetic construct
  source 1..321: mol_type = other DNA
gacattcagatgacccagtcaccagcatctttgagcgcatccgttggggagactgtgacaatcacatgccgaaccagtaagaacatcttcaacttcctcgcatggtaccatcaaaagcagggcaggtctcccagactgcttgtctctcacaccaagacactggcagcaggcgtccccagccggtttagtggtagtggatctggcacacagtttagtttgaaaatcaattccctgcaacccgaagacttcggcatatactattgccagcaccactatgggacaccttggactttcggaggtggtactaaacttgagattaaa

SEQ ID NO: 102 - DNA, 318 bp, synthetic construct
  source 1..318: mol_type = other DNA
caaataattctgtcacagtcccccgctatacttagtgcttcaccaggagaaaaagtgaccatgacttgtagagctaattctggcatatcattcatcaactggtatcaacaaaagccaggttcctcccccaagccatggatttacgggaccgccaaccttgcttctggggtacccgctcgtttcggcggatcaggttcaggaacttcctatagcctcactatcagtcgggttgaagctgaggatgccgctacatattactgccagcaatggtctagtaatccacttacctttggagctggcaccaaattggaacttcgt

SEQ ID NO: 103 - DNA, 321 bp, synthetic construct
  source 1..321: mol_type = other DNA
gacatccagatgacacagtcaccagcatccctgtccgcctcagttggggagcctgttaccataacttgtcgggcaagcaaaaacatatacacctatttggcttggtatcaccaaaagcaaggtaagtcacctcagtttcttgtatataatgcccgcacacttgctggcggagtaccctctcgattgtctggatctggcagcgttacccaattcagcctgaacatcaacaccctccatcgggaagatttgggtacctatttctgtcaacatcactacgacaccccatacaccttcggaggcggcacaaatttggaaattaaa

SEQ ID NO: 104 - DNA, 321 bp, synthetic construct
  source 1..321: mol_type = other DNA
gacatacaaatgacacaaagtcccgctagtctttcagccagtgttggtgagactgtgacaataacctgtagagctagcaaaaatgtctactcctatctggcttggttccagcagaaacaaggaaagagtcctcagttgctcgtatataatgctaaaactttggcagaaggcgtcccttctcgtttcagtggcggaggaagtgggactcaattctcactgaagatcaatagcctccagcccgccgactttgggagctactattgccaacatcattacaacataccattcacctttggctcaggtactaaactcgaaattaaa

SEQ ID NO: 105 - DNA, 330 bp, synthetic construct
  source 1..330: mol_type = other DNA
gacatagtgctcactcagagccctgcatcccttgccgcctccctcggacaacgagctactacaagctaccgggcatcaaagtccgttagcacatcaggatacagctatatgcactggaatcagcaaaagccaggccaaccaccccgtcttctcatctacctcgtaagtaatctggaatcaggcgtgccagcccgattcagtgggtcagggtctgggacagatttcaccctcaacatccatccagtagaggaagaggacgcagcaacatattactgccaacacattagagaacttaccactttcggaggaggaactaaattggagatcaaa

SEQ ID NO: 106 - DNA, 369 bp, synthetic construct
  source 1..369: mol_type = other DNA
cagatccagttggtgcagtctggacctgagctgaagaagcctggagagacagtcaagatctcctgcaaggcttctgggtattccttcagagactatggaatgaactgggtgaagcaggctccaggaaggacttttaagtggatgggctggataaacacctacactggagcgccagtatatgctgctgacttcaagggacggtttgccttctctttggacacctctgccagcgctgcctttttgcagatcaacaacctcaaaaatgaagacacggctacatatttctgtgcaagatgggtctcctacggtaataaccgcaactggttcttcgatttttggggcgcagggaccacggtcaccgtctcctca

SEQ ID NO: 107 - DNA, 354 bp, synthetic construct
  source 1..354: mol_type = other DNA
gaggtccagctgcaacagtctggacctgagctggtgaagcctggggcttcagtgaagatatcctgcaagacttctggatacacattcactgattacaccatgcactgggtgaggcagagccatggaaagagccttgagtggattggaagttttaatcctaacaatggtggtcttactaactacaaccagaagttcaagggcaaggccacattgactgtggacaagtcttccagcacagtgtacatggacctccgcagcctgacatctgaggattctgcagtctattactgtacaagaatccgggctacgggctttgactcctggggccagggcaccactctcacagtctcctca

SEQ ID NO: 108 - DNA, 348 bp, synthetic construct
  source 1..348: mol_type = other DNA
gaggtccaggtacagcagtctggacctgaactggtaaagcctggggcttcagtgaagatgtcctgtaaggcttctggatacacattcactagctatgtcatgcactgggtgaagcagaagcctgggcagggccttgagtggattggatatattaatcctaacaatgataatattaactacaatgagaagttcaaaggcaaggcctcactgacttcagacaaatcctccaacacagtctacatggagctcagcagcctgacctctgaggactctgcggtctattactgtgcaggctatggtaactccggagctaactggggccaagggactctggtcactgtctctgca

SEQ ID NO: 109 - DNA, 360 bp, synthetic construct
  source 1..360: mol_type = other DNA
cagatccagttggtgcagtctggacctgagctgaagaagcctggagagacagtcaagatctcctgcaaggcttctgggtataccttcacaaactatggagtgaagtgggtgaagcaggctccaggaaaggatttaaagtggatgggctggataaacacctacactggaaatccaatttatgctgctgacttcaagggacggtttgccttctctttggagacctctgccagcactgcctttttgcagatcaacaacctcaaaaatgaggacacggctacatatttctgtgtaagattccaatatggcgattaccggtacttcgatgtctggggcgcagggaccacggtcaccgtctcctca

SEQ ID NO: 110 - DNA, 348 bp, synthetic construct
  source 1..348: mol_type = other DNA
gaggtccagctgcagcagtctggacctgagctggtaaagcctggggcttcagtgaagctgtcctgcaaggcttctggatacacagtcactagctatgttatgcactgggtgaagcagaagcctgggcagggccttgagtggattggatatattaatccttacagtgatgttactaactgcaatgagaagttcaaaggcaaggccacactgacttcagacaaaacctccagcacagcctacatggagctcagcagcctgacctctgaggactctgcggtctattactgttcctcctacggtggggggtttgcttactggggccaagggactctggtcactgtctctgca

SEQ ID NO: 111 - DNA, 357 bp, synthetic construct
  source 1..357: mol_type = other DNA
cagatccagttggtgcagtctggacctgagctgaagaagcctggagagacagtcaagatctcctgcaaggcttctgggtttaccttcacaaactatccaatgcactgggtgaagcaggctccaggaaagggtttaaagtggatgggctggataaacacctactctggagtgccaacatatgcagatgacttcaagggacggtttgccttctctttggaaacctctgccagcactgcatatttgcagatcaacaacctcaaaaatgaggacatggctacatatttctgtgcaagagggggctacgatggtagcagggagtttgcttactggggccaagggactctggtcactgtctct

SEQ ID NO: 112 - DNA, 1362 bp, synthetic construct
  source 1..1362: mol_type = other DNA
tctaccggacaggtgcagttggttcagtctggcgccgaagtgaagaaacctggcgcttctgtgaaggtgtcctgcaaggcctctggctacacctttaccaactacggcgtgaaatgggtccgacaggctcctggacaggatctggaatggatgggctggatcaacacctacaccggcaatcctatctacgccgccgacttcaagggcagagtgaccatgaccaccgacacctctacctccaccgccttcatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 113 - DNA, 1353 bp, synthetic construct
  source 1..1353: mol_type = other DNA
gaagtgcagttggtgcagtctggcgccgaagtgaagaaacctggcgcttctgtgaaggtgtcctgcaaggcctctggctacacctttaccaactacggcgtgaaatgggtccgacaggctcctggacaaggcctggaatggatgggctggatcaacacctacaccggcaatcctatctacgccgccgacttcaagggcagagtgaccatgaccaccgacacctctacctccaccgcctacatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaattctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 114 - DNA, 1362 bp, synthetic construct
  source 1..1362: mol_type = other DNA
gaggtgcagctggttgaatctggcggaggacttgtgaagcctggcggctctctgagactgtcttgtgccgcctctggcttcaccttctccagctttgccatgtcctgggtccgacaggctcctggcaaaggactggaatgggtgtccaccatctcctccggctccatctacatctactacaccgacggcgtgaagggcagattcaccatcagcagagacaacgccaagaactccctgtacctgcagatgaacagcctgagagccgaggacaccgccgtgtactattgtgccagacggggcatctactatggctacgacggctacgctatggactattggggacagggcacactggtcaccgtgtcctctgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 115 - DNA, 336 bp, synthetic construct
  source 1..336: mol_type = other DNA
gatgttttgatgacccaaactccactctccctgcctgtccgtcttggagatcaggcctccatctcttgtagatctagtcagacccttgtacatagtaatggaaacacctatttagaatggtacctgcagaaaccaggccagtctccaaaactcttgatctacaaagtttccaatcgactttctggggtcccagacaggttcagtggcagtggatcagggacagatttcacactcaagatcagcagagtggagactgaggatctgggagtttattactgctttcaaggttcacatggtccgtggacgttcggtggaggcaccaagctggaaatcaaa

SEQ ID NO: 116 - DNA, 339 bp, synthetic construct
  source 1..339: mol_type = other DNA
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcaatgtgcaggctgaagacctgacagtttattactgcaagcaatcttataatcctccgtggacgttcggtggaggcaccaagctggaaatcaaa

SEQ ID NO: 117 - DNA, 321 bp, synthetic construct
  source 1..321: mol_type = other DNA
gacatccagatgactcagtctccagcctccctatctgcatctgtgggagaaactgtcaccatcacatgtcgaacaagtaaaaatattttcaattttttagcatggtatcaccagaaacagggaagatctcctcgactcctggtctctcatacaaaaaccttagcagcaggtgtgccatcaaggttcagtggcagtggctcaggcacacagttttctctgaagatcaacagcctgcagcctgaagattttgggatttattactgtcaacatcattatggtactccgtggacgttcggtggaggcaccaaactggaaatcaaa

SEQ ID NO: 118 - DNA, 318 bp, synthetic construct
  source 1..318: mol_type = other DNA
caaattattctctcccagtctccagcaatcctgtctgcatctccaggggagaaggtcacgatgacttgcagggccaactcaggtattagtttcatcaactggtaccagcagaagccaggatcctcccccaaaccctggatttatggcacagccaacctggcttctggagtccctgctcgcttcggtggcagtgggtctgggacttcttactctctcacaatcagcagagtggaggctgaagacgctgccacttattactgccagcagtggagtagtaacccgctcacgttcggtgctgggaccaagctggagttgaga

SEQ ID NO: 119 - DNA, 321 bp, synthetic construct
  source 1..321: mol_type = other DNA
gacatccagatgactcagtctccagcctccctatctgcatctgtgggagaacctgtcaccatcacatgtcgagcaagtaagaatatttacacatatttagcatggtatcaccagaaacagggaaaatctcctcagttcctggtctataatgcaagaaccttagcaggaggtgtgccatcaaggctcagtggcagtggatcagtcacgcagttttctctaaacatcaacaccttgcatcgagaagatttagggacttacttctgtcaacatcattatgatactccgtacacgttcggaggggggaccaacctggaaataaaa

SEQ ID NO: 120 - DNA, 321 bp, synthetic construct
  source 1..321: mol_type = other DNA
gacatccagatgactcagtctccagcctccctatctgcatctgtgggagaaactgtcaccatcacatgtcgagcaagtaaaaatgtttacagttatttagcatggtttcaacagaaacaggggaaatctcctcagctcctggtctataatgctaaaaccttagcagaaggtgtgccatcaaggttcagtggcgggggatcaggcacacagttttctctgaagatcaacagcctgcagcctgcagattttgggagttattactgtcaacatcattataatattccattcacgttcggctcggggacaaagttggaaataaaa

SEQ ID NO: 121 - DNA, 330 bp, synthetic construct
  source 1..330: mol_type = other DNA
gacattgtgctgacacagtctcctgcttccttagctgcatctctggggcagagggccaccacctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttaccacgttcggaggggggaccaagctggaaataaaa

SEQ ID NO: 122 - DNA, 714 bp, synthetic construct
  source 1..714: mol_type = other DNA
aagcttgccaccatggaaaccgacacactgctgctgtgggtgctgttgttgtgggtgccaggatctaccggagagatcatcctgacacagagccccgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagatttggcggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 123 - DNA, 651 bp, synthetic construct
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 124 - DNA, 651 bp, synthetic construct
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 125 - DNA, 651 bp, synthetic construct
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 126 - DNA, 651 bp, synthetic construct
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 127 - DNA, 660 bp, synthetic construct
  source 1..660: mol_type = other DNA
gccatcgtgttgacccagtctccaggcacattgtctctgagccctggcgagagagctaccctgtcctgcagatcttctcagtccctggtgcactccaacggcaacacctacctgcactggtacatgcagaagcccggacaggctcccagagtgctgatctacaaggtgtccaaccggttctctggcatccccgacagattttccggctctggctctggcaccgacttcaccctgaccatctctagactggaacccgacgacttcgccatctactactgctcccagtccacacacatccctctggcttttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggcctgtctagccctgtgaccaagtctttcaaccggggcgagtgttga

SEQ ID NO: 128 - DNA, 11303 bp, synthetic construct
  source 1..11303: mol_type = other DNA
gaggtgatcctggtcgagagtggaggtgggttggttaagcccggaggtagcctgaagttatcctgtgccgccagcggctttactttcagttctttcgctatgtcctgggtgcgccaaacgccggaaaagcggctggagtgggtggctaccatctcatccgggagcatttatatatattatacagatggtgtgaaaggccgatttaccatcagccgggacaacgcgaagaacaccgtccacctgcaaatgtcttctttacgatcagaagacacagccatgtactactgtgctaggcgaggaatctattatggctatgatgggtacgccatggattattggggccagggcaccagtgtgaccgtctcaggcggcggcggatccggaggaggaggcagcgggggcggcggttccgctgtggtgatgactcaaacgcccctctccctgcctgtctcactcggggaccaggcttccattagttgcaggagcagccagtctttagtgcactccaacggcaacacatatctgcattggtatatgcaaaaaccaggacaatcccctaaagtgctcatttacaaagtgtcaaaccgcttcagcggagtgcccgacagattttccggctcaggctcagggaccgacttcactttaaagatttccagagtggaagccgacgatctggggatctacttctgctcccaaagcactcatattcccctggccttcggagctggaactaaactggaactcaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctccctaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgggaagcgggtccggtagcggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatctggagctctcgagaattctcacgcgtcaagtggagcaaggcaggtggacagtgatggccttaccagtgaccgccttgctcctgccgctggccttgctgctccacgccgccaggccggagcagaagctgatcagcgaggaggacctggaggaggacctg

SEQ ID NO: 129 - DNA, 11303 bp, synthetic construct
  source 1..11303: mol_type = other DNA
gcagttgtcatgactcagaccccattaagcctccctgtctcattaggcgaccaggccagcatttcatgtcgctcctcacaaagcctggtgcattccaacggcaacacatatctgcactggtacatgcaaaaaccaggccagagtccaaaggtcctgatctacaaggtgtccaacagattcagtggagtccccgataggttttctgggagcggctcaggtacagacttcacgctgaaaatcagcagagttgaagccgacgacctggggatctacttctgtagccagtccacacatatccccttggcgtttggcgccggaacgaagctcgaactgaaagggggtggaggcagcggaggggggggctcaggcggaggcggcagcgaagtaatcctcgtggaaagtggggggggcctggtaaagccgggcggcagcctcaagttatcatgcgctgccagcggattcaccttcagctccttcgccatgtcctgggtccgtcagactcccgagaagcgcctcgaatgggtcgccaccatctctagcggctcaatatacatttattacactgacggagtcaagggtcgcttcactattagccgggacaacgcaaaaaacactgtacatctgcagatgtcatccctgcggtccgaagacactgctatgtactactgcgcaagacggggtatctactacggctatgatggctacgccatggactattggggccaggggacctctgttaccgtgtctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctccctaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgggaagcgggtccggtagcggatctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttgacttgcggccgcaactcccacctgcaacatgcgtgactgactgaggccgcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatctggagctctcgagaattctcacgcgtcaagtggagcaaggcaggtggacagtgatggccttaccagtgaccgccttgctcctgccgctggccttgctgctccacgccgccaggccggagcagaagctgatcagcgaggaggacctggaggaggacctg

SEQ ID NO: 130 - protein, 339 aa, synthetic construct
GAGGTCCAGCTGCAGCAGTCTGGACCTGAGCTGGTAAAGCCTGGGGCTTCAGTGAAGATGTCCTGCAAGGCTTCTGGATACACATTCTCTAGCTATGTTATGCACTGGGTGAAGCAGAAGCCTGGGCAGGGCCTTGAGTGGATTGGATATATTAATCCTTACAGTGATGTCACTAACTACAATGAGAAGTTCAAAGGCAAGGCCACACTGACTTCAGACAGATCCTCCAACACAGCCTACATGGAACTCAGCAGCCTGACCTCTGAGGACTCTGCGGTCTATTACTGTGCAAGAAATTACTTCGACTGGGGCCGAGGGACTCTGGTCACAGTCTCTGCA

(19) United States
(12) Patent Application Publication
(10) Pub. No.: US 2025/0352648 A1
(43) Pub. Date: Nov. 20, 2025

(54) TM4SF1 CAR CELLS AND METHODS OF USE THEREOF

(71) Applicants: The United States Government As Represented By The Department Of Veterans Affairs, Washington, DC (US); The Regents Of The University Of California, Oakland, CA (US)

(72) Inventors: Franklin W. Huang, San Francisco, CA (US); Sima P. Porten, San Francisco, CA (US)

(21) Appl. No.: 19/207,804

(22) Filed: May 14, 2025

Related U.S. Application Data
(60) Provisional application No. 63/649,157, filed on May 17, 2024.
(60) Provisional application No. 63/649,821, filed on May 20, 2024.

(51) Int. Cl. A61K 40/42; A61K 35/00; A61K 40/11; A61K 40/31; C07K 14/725; C07K 16/28

(52) U.S. Cl. CPC A61K 40/4224; A61K 35/00; A61K 40/11; A61K 40/31; C07K 14/7051; C07K 16/2896; C07K 2317/622

(57) ABSTRACT

Disclosed are CAR polypeptides comprising a TM4SF1 antigen binding domain, a transmembrane domain, and an intracellular signaling domain. Disclosed are nucleic acid sequences capable of encoding a CAR polypeptide comprising a TM4SF1 antigen binding domain, a transmembrane domain, and an intracellular signaling domain. Disclosed are methods of treating bladder cancer comprising administering a therapeutically effective amount of a composition comprising a T cell genetically modified to express one or more of the CAR polypeptides disclosed herein to a subject in need thereof. Disclosed are methods of killing TM4SF1 positive cells comprising administering an effective amount of a T cell genetically modified to express a CAR polypeptide comprising a TM4SF1 antigen binding domain, a hinge and transmembrane domain, and an intracellular signaling domain.

### CROSS-REFERENCE TO RELATED APPLICATIONS

This application claims the benefit of U.S. Provisional Patent Application Nos. 63/649,157, filed May 17, 2024, and 63/649,821, filed May 20, 2024 each of which is incorporated by reference herein in its entirety.

### REFERENCE TO SEQUENCE LISTING

The Sequence Listing submitted May 14, 2025 as a text file named “37759.0637U3.xml,” created on Apr. 28, 2025, and having a size of 158,436 bytes is hereby incorporated by reference pursuant to 37 C.F.R. § 1.52(e)(5).

### BACKGROUND

Histologic variant (HV) subtypes of bladder cancer are found in up to 25% of all bladder tumors. Compared to bladder tumors with pure urothelial carcinoma (UC) histology, tumors with HVs are associated with worse clinical outcomes. Many HV subtypes do not respond well to systemic therapy, so the clinical management for HVs often diverges from the treatment guidelines tailored for pure UC cancers. The limited treatment options presents an immense challenge for patients and providers.

While significant progress has been made to define the molecular characteristics of pure UC, much less is known about the biology of HVs. It remains unclear whether each HV subtype should be regarded as a distinct entity or whether HVs share common features as a group. Some genomic alterations, such as TERT promoter mutations in micropapillary, plasmacytoid, and adenocarcinoma variants, appear to be more associated with HVs than UCs, while others, such as CDH1 truncations in plasmacytoid variants, appear to be subtype defining. The existing evidence does not suggest that HV biology is governed solely at the genomic level. Transcriptional analyses based on bulk RNA sequencing data have not revealed clinically useful insights, however. This use of bulk RNA sequencing is not well suited to study HVs because it requires large sample sizes that are difficult to achieve in HVs, especially when considering the number of individual subtypes.

### BRIEF SUMMARY

To better understand the biology of HVs and to identify exploitable molecular features, single cell RNA sequencing (scRNA-seq) was used to transcriptionally profile HV-containing bladder tumors. This approach allowed for the unveiling of a CA125+ cell state with adverse features specific to HVs and TM4SF1 as broadly enriched surface antigen in HVs that can be targeted with CAR T cells.

Disclosed are CAR polypeptides comprising a TM4SF1 antigen binding domain, a transmembrane domain, and an intracellular signaling domain.

Disclosed are nucleic acid sequences capable of encoding a CAR polypeptide comprising a TM4SF1 antigen binding domain, a transmembrane domain, and an intracellular signaling domain.

Disclosed are methods of treating bladder cancer comprising administering a therapeutically effective amount of a composition comprising a T cell genetically modified to express one or more of the CAR polypeptides disclosed herein to a subject in need thereof.

Disclosed are methods of killing TM4SF1 positive cells comprising administering an effective amount of a T cell genetically modified to express a CAR polypeptide comprising a TM4SF1 antigen binding domain, a hinge and transmembrane domain, and an intracellular signaling domain.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1F show top level clustering analysis of tumor epithelial cells and characterization of a common tumor cluster. (FIG. 1A) Clustering UMAPs of tumor epithelial cells (N=8,553) extracted from the main dataset color-coded by cluster and annotated according to tumor ID. Cluster 13 (ellipse) is annotated separately due to contributions from multiple tumors. (FIG. 1B) Barchart of cluster composition by patient/tumor. (FIG. 1C) Table displaying primary and secondary histologic patterns observed in each tumor. (FIG. 1D) Curated dot plot of top differentially expressed genes (DEGs) by tumor cluster. (FIG. 1E) Immunohistochemistry of CA125 in primary tumor cells (VAR05) and nodal metastases (VAR09). Scale bar=50 μm. (F) Preoperative serum CA125 values in patients with UC and HV bladder tumors.

FIGS. 2A-2D show transcriptional relationship between Cluster 13 and parent tumor cells. (FIG. 2A) Partition-based graphical abstraction of tumor cell clusters. (FIG. 2B) Dot plot of tumor signature scores relative to Cluster13 tumors of origin. (FIG. 2C) UMAP of individual tumors color-coded by Cluster 13 cells (red) and pseudotime using Cluster 13 cells as the starting point. (FIG. 2D) Expression along the pseudotime of Cluster 13 and parent tumor DEGs.

FIGS. 3A-3D show cluster 13 is associated with metastasis and chemotherapeutic resistance. (FIG. 3A). Gene ontology analysis of Cluster 13 gene signature. (FIG. 3B) CA125 immunohistochemistry in a primary HV bladder tumor and the corresponding lymph node metastasis. (FIG. 3C) Drug susceptibility heatmap for gene signature individual tumor clusters and average UC and HV profiles. (FIG. 3D) Kaplan-Meier curves of overall and disease-specific survival according to Cluster 13 signature enrichment in TCGA-BLCA.

FIGS. 4A-4I show nonurothelial transcriptional programs in VAR09 and VAR08. (FIG. 4A) Feature plots of small cell lung cancer (SCLC) molecular subtype-defining genes ASCL1 (SCLC-A), NEUROD1 (SCLC-N), POU2F3 (SCLC-P), and YAP1 (SCLC-Y) expression in VAR09. (FIG. 4B) Expression of POU2F3 downstream targets across tumor clusters. (FIG. 4C) UMAP of VAR09 color-coded by subcluster and pseudotime using subcluster 4 as starting point. (FIG. 4D) Urothelial stemness signature score among VAR09 subclusters. (FIG. 4E) Expression of KRT7 and POU2F3 along the VAR09 pseudotime. (FIG. 4F) Schematic of HOXB genes and transcription factors IRF4, PRDM1, and XBP1 along the plasma cell lineage. (FIG. 4G) Expression of plasma cell-related genes among tumor clusters. (FIG. 4H) Feature plots of HOXB4, HOXB3, PRDM1, and IL6R expression in VAR08 cells. (FIG. 4I) Expression of urothelial and lymphoid genes along the VAR08 pseudotime.

FIGS. 5A-F show the identification of TM4SF1 as a gene enriched in HVs. (FIG. 5A) Volcano plot comparison of all UC and HV cells after downsampling (N=150 per patient). (FIG. 5B) Violin plots of TM4SF1 expression by tumor cluster. (FIG. 5C) Correlation plots of TM4SF1 and EMP1, EZR, CLDN4, and KRT19. (FIG. 5D-FIG. 5E) Immunohistochemistry of TM4SF1 in a validation cohort of HV and UC (FIG. 5D) primary tumors and FIG. 5E lymph node metastases. (FIG. 5F) Semiquantitative comparison of TM4SF1 staining in HV and UCs.

FIGS. 6A-6F show an evaluation of TM4SF1 CAR T cell activity. (FIG. 6A) Schematic for generating TM4SF1-CAR T cells. (FIG. 6B) Bladder cancer cell lines and TM4SF1 expression determined by flow cytometric fluorescent antibody detection and mRNA expression. (FIG. 6C) Quantification of in vitro TM4SF1-CAR1 and CAR2 activity against bladder cancer cell lines using IncuCyte. (FIG. 6D) Schematic for xenograft generation from the UMUC3 cell line and in vivo TM4SF1-CAR1 testing. (FIG. 6E) Tumor size comparisons between TM4SF1-CAR treated and untreated mice. (FIG. 6F) Kaplan-Meier survival analysis of treated and untreated mice.

FIG. 7 shows representative H&E stains from each sequenced tumor.

FIGS. 8A-8B show single cell dataset of variant and pure urothelial tumor epithelial cells. FIG. 8A) Tissue acquisition and scRNA-seq workflow for primary bladder tumors. FIG. 8B) UMAP of full dataset color-coded by broad cell type. FIG. 8C) Bar chart of cell counts obtained from each patient/tumor.

FIGS. 9A-9B show annotation of cell types and confirmation of tumor content. FIG. 9A) Feature plots of genes used to determine top-level annotations for epithelial cells (EPCAM, KRT7), immune cells (PTPRC), fibroblasts (DCN), smooth muscle (ACTA2), and endothelial cells (SELE). FIG. 9B) InferCNV analysis of all tumor epithelial cells using tumor microenvironment components for comparison.

FIG. 10 shows negative CA125 (MUC16) staining in variant and pure UC tumor components. Scale bar=50 μm

FIGS. 11A-11B show detection of Cluster 13 signature in an external scRNAseq data set (Chen et al.). FIG. 11A) Feature plot of Cluster 13 signature gene set enrichment. FIG. 11B) Feature plot demonstrating expression of individual cluster 13-defining genes.

FIG. 12 shows enrichment of bladder cancer stem cell signature (PROM1 (CD133), POU5F1 (Oct4), SOX2, ALDH1A1, SOX4, EZH2, YAP1, CD44, and KRT14 along the pseudotime for VAR01, VAR03, VAR05, VAR06, and VAR07).

FIGS. 13A-13B show assessment of urothelial carcinoma gene signature in each tumor. FIG. 13A) Expression of urothelial carcinoma gene signature (Mo et al.) in each tumor by individual gene. FIG. 13B) Urothelial carcinoma signature enrichment in each tumor.

FIG. 14 shows assessment of HOX gene expression among variant subclusters.

FIGS. 15A-15C show immune cell annotation and signature enrichment in tumor cells. FIG. 15A) Clustering UMAP of immune cells with top-tier annotations. FIG. 15B) Heat map demonstrating Top 5 DEGs in each immune cluster. FIG. 15C) Enrichment of immune cell signatures within each tumor.

FIGS. 16A-16B show plasma cell transcriptional programs in tumor cells. FIG. 16A) Dot plot showing expression of top 100 plasma cell genes in each tumor. FIG. 16B) Gene set enrichment analysis of protein secretion and unfolded protein response in VAR08 tumor cells.

FIGS. 17A-17C show association of TM4SF1 with clinical features and luminal-basal subtypes in TCGA-BLCA. FIG. 17A) Single set gene set enrichment analysis (ssGSEA) for TM4SF1 in TCGA-BLCA. FIG. 17B) TM4SF1 expression stratified by grade, stage, and lymph node status. FIG. 17C) Kaplan-Meier curves showing overall survival according to TM4SF1 expression (pink=high, blue=low) in urothelial cancers, renal cancers, and pancreatic cancers.

FIGS. 18A-18D show genes correlated with TM4SF1. FIG. 18A) Scatter plot showing correlation coefficient and expression of genes associated with TM4SF1. FIGS. 18B-C) Correlation plots between TM4SF1 and CLDN4, EZR, EMPT, and KRT19 with linear regression within (18B) tumor epithelial dataset and (18C) by tumor. FIG. 18D) ssGSEA in TCGA0BLCA between TM4SF1 and EMP1, CLDN4, EZR, and KRT19.

FIG. 19 shows correlation between NECTIN4 and TM4SF1 in CCLE.

FIG. 20 shows IncuCyte growth curve of UMUC3 cell line after TM4SF1 knockdown using CRISPR-Cas9. UMUC3 TM4SF1-KO cells were treated with no T cells, both CARs in a 2:1 effector:tumor cell ratio, and untransduced T-cells.

FIG. 21 shows a schematic for xenograft experiments.

FIG. 22 shows body weights of TM4SF1-CAR T treated mice.

FIG. 23 is a table showing clinical and pathologic characteristics of patients and sequenced bladder cancer tissues.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a TM4SF1 antigen binding domain” includes a plurality of such TM4SF1 antigen binding domains, reference to “the TM4SF1 antigen binding domain” is a reference to one or more TM4SF1 antigen binding domains and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” bladder cancer may refer to inhibiting survival, growth, and/or spread of the cancer cells. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Chimeric Antigen Receptor (CAR) Polypeptide

Disclosed are CAR polypeptides comprising a TM4SF1 antigen binding domain, a transmembrane domain, and an intracellular signaling domain. In some aspects, the TM4SF1 antigen binding domain, transmembrane domain, and intracellular signaling domain can be any of those described herein and any combination of those described herein. For example, disclosed are CAR polypeptides, wherein the CAR polypeptide comprises a TM4SF1 antigen binding domain comprising a heavy chain variable domain comprising a CDR3 domain comprising an amino acid sequence that has at least 75% identity to SEQ ID NO: 1, 2, 3, 4, 5, 6, 7, 8, 9, 10, 11, or 12; a CDR2 domain comprising an amino acid sequence that has at least 75% identity to SEQ ID NO: 13, 14, 15, 16, 17, 18, 19, 20, 21, 22; and a CDR1 domain comprising an amino acid sequence that has at least 75% identity to SEQ ID NO: 23, 24, 25, 26, 27, 28, 29, 30, 31; and a light chain variable domain comprising a CDR3 domain comprising an amino acid sequence that has at least 75% identity to SEQ ID NO: 32, 33, 34, 35, 36, 37, 38, 39, 40; a CDR2 domain comprising an amino acid sequence that has at least 75% identity to SEQ ID NO: 41, 42, 43, 44, 45, 46, 47, 48, 49; and a CDR1 comprising an amino acid sequence that has at least 75% identity to SEQ ID NO: 50, 51, 52, 53, 54, 55, 56, 57, 58, 59, 60, 61, 62; IgG4 spacers, a CD8 hinge, a CD8 transmembrane domain, a 4-1BB costimulatory domain; and a CD3ζ chain.

### 1. TM4SF1 Antigen Binding Domain

In some instances, the TM4SF1 antigen binding domain can be an antibody fragment or an antigen-binding fragment that specifically binds to TM4SF1. In some instances, the TM4SF1 antigen binding domain can be any recombinant or engineered protein domain capable of binding TM4SF1.

In some instances, the TM4SF1 antigen binding domain can be a Fab or a single-chain variable fragment (scFv) of an antibody that specifically binds TM4SF1. In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some instances, the TM4SF1 antigen binding domain comprises an amino acid sequence set forth in SEQ ID NO: 86 or 87. In some instances, the TM4SF1 antigen binding domain can comprise a heavy chain variable region, a light chain variable region, and a linker that links the heavy chain variable region to the light chain variable region. For example, SEQ ID NOs: 86 and 87 comprise the heavy chain variable region, linker, and light chain variable region (see Table 1). In some instances, the linker can be directly involved in the binding of TM4SF1 to the TM4SF1 antigen binding domain. In some instances, the linker can be indirectly involved in the binding of TM4SF1 to the TM4SF1 antigen binding domain.

TABLE 1
TM4SF1 antigen binding domains. Variable heavy chain (bold),
linker (underlined), and variable light chain

| SEQ ID NO: | |
|---|---|
| SEQ ID | EVILVESGGGLVKPGGSLKLSCAASGFTFSSFAMSWVRQTPEKRL |
| NO: 86 | EWVATISSGSIYIYYTDGVKGRFTISRDNAKNTVHLQMSSLRSEDT |
| | AMYYCARRGIYYGYDGYAMDYWGQGTSVTVSGGGGSGGGGSGG |
| | GGSAVVMTQTPLSLPVSLGDQASISCRSSQSLVHSNGNTYLHWYMQK |
| | PGQSPKVLIYKVSNRFSGVPDRFSGSGSGTDFTLKISRVEADDLGIYFC |
| | SQSTHIPLAFGAGTKLELK |
| SEQ ID | AVVMTQTPLSLPVSLGDQASISCRSSQSLVHSNGNTYLHWYMQKPGQ |
| NO: 87 | SPKVLIYKVSNRFSGVPDRFSGSGSGTDFTLKISRVEADDLGIYFCSQST |
| | HIPLAFGAGTKLELKGGGGSGGGGSGGGGSEVILVESGGGLVKPGGS |
| | LKLSCAASGFTFSSFAMSWVRQTPEKRLEWVATISSGSIYIYYTDG |
| | VKGRFTISRDNAKNTVHLQMSSLRSEDTAMYYCARRGIYYGYDG |
| | YAMDYWGQGTSVTVSS |

In some instances, the TM4SF1 antigen binding domain comprises a variable heavy chain comprising a sequence having at least 90% identity to a sequence set forth in SEQ ID NOs:88, 63, 64, 65, 66, 67, 68, 69, 70, 71, 72, or 73 (See Table 2). In some instances, the TM4SF1 antigen binding domain comprises a variable heavy chain comprising a sequence having at least 90, 91, 92, 93, 94, 95, 96, 97, 98, 99, or 100% identity to a sequence set forth in SEQ ID NOs: 88, 63, 64, 65, 66, 67, 68, 69, 70, 71, 72, or 73.

TABLE 2
Variable Heavy Chains

| SEQ ID | |
|---|---|
| NO: | Amino Acid Sequence |
| 88 | EVILVESGGGLVKPGGSLKLSCAASGFTFSSFAMSWVRQTPEKRLEWVAT |
| | ISSGSIYIYYTDGVKGRFTISRDNAKNTVHLQMSSLRSEDTAMYYCARRGI |
| | YYGYDGYAMDYWGQGTSVTVS |
| 63 | QIQLVQSGPELKKPGETVKISCKASGYSFRDYGMNWVKQAPGRTFKWM |
| | GWINTYTGAPVYAADFKGRFAFSLDTSASAAFLQINNLKNEDTATYFCAR |
| | WVSYGNNRNWFFDFWGAGTTVTVSS (3) |
| 64 | EVQLQQSGPELVKPGASVKISCKTSGYTFTDYTMHWVRQSHGKSLEWIG |
| | SFNPNNGGLTNYNQKFKGKATLTVDKSSSTVYMDLRSLTSEDSAVYYCT |
| | RIRATGFDSWGQGTTLTVSS (15) |
| 65 | EVQVQQSGPELVKPGASVKMSCKASGYTFTSYVMHWVKQKPGQGLEWI |
| | GYINPNNDNINYNEKFKGKASLTSDKSSNTVYMELSSLTSEDSAVYYCAG |
| | YGNSGANWGQGTLVTVSA (27) |
| 66 | QIQLVQSGPELKKPGETVKISCKASGYTFTNYGVKWVKQAPGKDLKWM |
| | GWINTYTGNPIYAADFKGRFAFSLETSASTAFLQINNLKNEDTATYFCVRF |
| | QYGDYRYFDVWGAGTTVTVSS (39) |
| 67 | EVQLQQSGPELVKPGASVKLSCKASGYTVTSYVMHWVKQKPGQGLEWI |
| | GYINPYSDVTNCNEKFKGKATLTSDKTSSTAYMELSSLTSEDSAVYYCSS |
| | YGGGFAYWGQGTLVTVSA (51) |
| 68 | EVQLQQSGPELVKPGASVKMSCKASGYTFSSYVMHWVKQKPGQGLEWI |
| | GYINPYSDVTNYNEKFKGKATLTSDRSSNTAYMELSSLTSEDSAVYYCAR |
| | NYFDWGRGTLVTVSA (63) |
| 69 | QIQLVQSGPELKKPGETVKISCKASGFTFTNYPMHWVKQAPGKGLKWMG |
| | WINTYSGVPTYADDFKGRFAFSLETSASTAYLQINNLKNEDMATYFCARG |
| | GYDGSREFAYWGQGTLVTVS (75) |
| 70 | QVQLVQSGAEVKKPGASVKVSCKASGYTFTNYGVKWVRQAPGQDLEW |
| | MGWINTYTGNPIYAADFKGRVTMTTDTSTSTAFMELRSLRSDDTAVYYC |
| | VRFQYGDYRYFDVWGQGTLVTVSSASTKGPSVFPLAPSSKSTSGGTAALG |
| | CLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPEAAGAPSVFLFPP |
| | KPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQP |
| | REPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPGK (90) |
| 71 | EVOLVQSGAEVKKPGASVKVSCKASGYTFTNYGVKWVRQAPGQGLEW |
| | MGWINTYTGNPIYAADFKGRVTMTTDTSTSTAYMELRSLRSDDTAVYYC |
| | VRFQYGDYRYFDVWGQGTLVTVSSASTKGPSVFPLAPSSKSTSGGTAALG |
| | CLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPEAAGAPSVFLFPP |
| | KPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQP |
| | REPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPGK (92) |
| 72 | EVOLVESGGGLVKPGGSLRLSCAASGFTFSSFAMSWVRQAPGKGLEWVS |
| | TISSGSIYIYYTDGVKGRFTISRDNAKNSLYLQMNSLRAEDTAVYYCARR |
| | GIYYGYDGYAMDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPEAAGAPSVFLF |
| | PPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKG |
| | QPREPQVYTLPPSREEMTKNQVSLTCLVKGFYPSDIAVEWESNGQPENNY |
| | KTTPPVLDSDGSFFLYSKLTVDKSRWQQGNVFSCSVMHEALHNHYTQKS |
| | LSLSPGK (112) |
| 73 | EVOLVESGGGLVKPGGSLRLSCAASGFTFSSFAMSWVRQAPGKGLEWVS |
| | TISSGSIYIYYTDSVKGRFTISRDNAKNSLYLQMNSLRAEDTAVYYCARRG |
| | IYYGYEGYAMDYWGQGTLVTVSSASTKGPSVFPLAPSSKSTSGGTAALG |
| | CLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPEAAGAPSVFLFPP |
| | KPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQP |
| | REPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPGK (114) |

In some instances, the TM4SF1 antigen binding domain comprises a variable light chain comprising a sequence having at least 90% identity a sequence set forth in SEQ ID NOs:74, 75, 76, 77, 78, 79, 80, 81, 82, 83, 84, or 85 (see Table 3). In some instances, the TM4SF1 antigen binding domain comprises a variable light chain comprising a sequence having at least 90, 91, 92, 93, 94, 95, 96, 97, 98, 99, or 100% identity to a sequence set forth in SEQ ID NOs: 74, 75, 76, 77, 78, 79, 80, 81, 82, 83, 84, or 85.

TABLE 3
Variable Light Chains.

| SEQ ID | |
|---|---|
| NO: | |
| 74 | AVVMTQTPLSLPVSLGDQASISCRSSQSLVHSNGNTYLHWYMQKPGQSPKVLIYKVSNRF |
| | SGVPDRFSGSGSGTDFTLKISRVEADDLGIYFCSQSTHIPLAFGAGTKLELK |
| 75 | DVLMTQTPLSLPVRLGDQASISCRSSQTLVHSNGNTYLEWYLQKPGQSPKLLIYKVSNRL |
| | SGVPDRFSGSGSGTDFTLKISRVETEDLGVYYCFQGSHGPWTFGGGTKLEIK (9) |
| 76 | DIVMSQSPSSLAVSAGEKVTMSCKSSQSLLNSRTRKNYLAWYQQKPGQSPKLLIYWASTR |
| | ESGVPDRFTGSGSGTDFTLTISNVQAEDLTVYYCKQSYNPPWTFGGGTKLEIK (21) |
| 77 | DIQMTQSPASLSASVGETVTITCRTSKNIFNFLAWYHQKQGRSPRLLVSHTKTLAAGVPS |
| | RFSGSGSGTQFSLKINSLQPEDFGIYYCQHHYGTPWTFGGGTKLEIK (33) |
| 78 | QIILSQSPAILSASPGEKVTMTCRANSGISFINWYQQKPGSSPKPWIYGTANLASGVPAR |
| | FGGSGSGTSYSLTISRVEAEDAATYYCQQWSSNPLTFGAGTKLELR (45) |
| 79 | DIQMTQSPASLSASVGEPVTITCRASKNIYTYLAWYHQKQGKSPQFLVYNARTLAGGVPS |
| | RLSGSGSVTQFSLNINTLHREDLGTYFCQHHYDTPYTFGGGTNLEIK (57) |
| 80 | DIQMTQSPASLSASVGETVTITCRASKNVYSYLAWFQQKQGKSPQLLVYNAKTLAEGVPS |
| | RFSGGGSGTQFSLKINSLQPADFGSYYCQHHYNIPFTFGSGTKLEIK (69) |
| 81 | DIVLTQSPASLAASLGQRATTSYRASKSVSTSGYSYMHWNQQKPGQPPRLLIYLVSNLES |
| | GVPARFSGSGSGTDFTLNIHPVEEEDAATYYCQHIRELTTFGGGTKLEIK (81) |
| 82 | EIILTQSPATLSLSPGERATLSCRANSGISFINWYQQKPGQAPRLLIYGTANLASGIPAR |
| | FGGSGSGRDFTLTISSLEPEDFAVYYCQQWSSNPLTFGGGTKVEIKRTVAAPSVFIFPPS |
| | DEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (97) |
| 83 | EIVLTQSPATLSLSPGERATLSCRANSGISFINWYQQKPGQAPRLLIYGTANLASGIPAR |
| | FSGSGSGRDFTLTISSLEPEDFAVYYCQQWSSNPLTFGGGTKVEIKRTVAAPSVFIFPPS |
| | DEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (99) |
| 84 | EIVLTQSPATLSLSPGERATLSCRAQSGISFINWYQQKPGQAPRLLIYGTANLASGIPAR |
| | FSGSGSGRDFTLTISSLEPEDFAVYYCQQWSSNPLTFGGGTKVEIKRTVAAPSVFIFPPS |
| | DEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (101) |
| 85 | AIVLTQSPGTLSLSPGERATLSCRSSQSLVHSNGNTYLHWYMQKPGQAPRVLIYKVSNRF |
| | SGIPDRFSGSGSGTDFTLTISRLEPDDFAIYYCSQSTHIPLAFGQGTKLEIKRTVAAPSV |
| | FIFPPSDEQLKSGTASVVCLLNNFYPREAKVQWKVDNALQSGNSQESVTEQDSKDSTYSL |
| | SSTLTLSKADYEKHKVYACEVTHQGLSSPVTKSFNRGEC (122) |

In some instances, the TM4SF1 antigen binding domain comprises a heavy chain immunoglobulin variable region comprising a complementarity determining region 1 (CDR1) comprising the sequence of SEQ ID NO:23, 24, 25, 26, 27, 28, 29, 30, 31; a CDR2 comprising the sequence of SEQ ID NO:13, 14, 15, 16, 17, 18, 19, 20, 21, 22; and a CDR3 comprising the sequence of SEQ ID NO:1, 2, 3, 4, 5, 6, 7, 8, 9, 10, 11, or 12.

TABLE 4
CDRs present in the heavy chain

| CDRs | | | |
|---|---|---|---|
| present in | | | |
| the heavy | | | |
| chain of | | | |
| SEQ ID NOs: | CDR1 | CDR2 | CDR3 |
| SEQ ID | GYSFRDYGMN | WINTYTGAPVYAADFKG | WVSYGNNRNWFFDF |
| NO: 63 | (SEQ ID NO: 23) | (SEQ ID NO: 13) | (SEQ ID NO: 1) |
| SEQ ID | GYTFTDYTMH | SFNPNNGGLTNYNQKFKG | IRATGFDS |
| NO: 64 | (SEQ ID NO: 24) | (SEQ ID NO: 14) | (SEQ ID NO: 2) |
| SEQ ID | GYTFTSYVMH | YINPNNDNINYNEKFKG | YGNSGAN |
| NO: 65 | (SEQ ID NO: 25) | (SEQ ID NO: 15) | (SEQ ID NO: 3) |
| SEQ ID | GYTFTNYGVK | WINTYTGNPIYAADFKG | FQYGDYRYFDV |
| NO: 66, | (SEQ ID NO: 26) | (SEQ ID NO: 16) | (SEQ ID NO: 4) |
| 70, 71 | | | |
| SEQ ID | GYTVTSYVMH | YINPYSDVTNCNEKFKG | YGGGFAY |
| NO: 67 | (SEQ ID NO: 27) | (SEQ ID NO: 17) | (SEQ ID NO: 5) |
| SEQ ID | GYTFSSYVMH | YINPYSDVTNYNEKFKG | NYFD |
| NO: 68 | (SEQ ID NO: 28) | (SEQ ID NO: 18) | (SEQ ID NO: 6) |
| SEQ ID | GFTFTNYPMH | WINTYSGVPTYADDFKG | GGYDGSREFAY |
| NO: 69 | (SEQ ID NO: 29) | (SEQ ID NO: 19) | (SEQ ID NO: 7) |
| SEQ ID | GYTFTNYGVK | WINTYTGNPIYAADFK | FQYGDYRYFDV |
| NO: 70 | (SEQ ID NO: 30) | (SEQ ID NO: 20) | (SEQ ID NO: 8) |
| SEQ ID | GFTFSSFAMS | TISSGSIYIYYTDGVKG | RGI |
| NO: 71 | (SEQ ID NO: 31) | (SEQ ID NO: 21), or | YYGYDGYAMDY |
| | | TISSGSIYIYYTDSVKG | (SEQ ID NO: 9), |
| | | (SEQ ID NO: 22) | RGI |
| | | | YYGYEGYAMDY |
| | | | (SEQ ID NO: 10), |
| | | | RGI |
| | | | YYGYSGYAMDY |
| | | | (SEQ ID NO: 11), or |
| | | | RGI |
| | | | YYGYAGYAMDY |
| | | | (SEQ ID NO: 12) |

In some instances, the TM4SF1 antigen binding domain comprises a light chain immunoglobulin variable region comprising a CDR1 comprising the sequence of SEQ ID NO:50, 51, 52, 53, 54, 55, 56, 57, 58, 59, 60, 61, 62; a CDR2 comprising the sequence of SEQ ID NO:41, 42, 43, 44, 45, 46, 47, 48, 49; and a CDR3 comprising the sequence of SEQ ID NO:32, 33, 34, 35, 36, 37, 38, 39, 40.

TABLE 5
CDRs present in the light chain

| CDRs | | | |
|---|---|---|---|
| present in | | | |
| the light | | | |
| chain of | | | |
| SEQ ID NOs: | CDR1 | CDR2 | CDR3 |
| SEQ ID | RSSQTLVHSNGNTYLE | KVSNRLS | FQGSHGPWT |
| NO: 75 | (SEQ ID NO: 50) | (SEQ ID NO: 41) | (SEQ ID NO: 32) |
| SEQ ID | KSSQSLLNSRTRKNYLA | WAS TRES | KQSYNPPWT |
| NO: 76 | (SEQ ID NO: 51) | (SEQ ID NO: 42) | (SEQ ID NO: 33) |
| SEQ ID | RTSKNIFNFLA | HTKTLAA | QHHYGTPWT |
| NO: 77 | (SEQ ID NO: 52) | (SEQ ID NO: 43) | (SEQ ID NO: 34) |
| SEQ ID | RANSGISFIN | GTANLAS | QQWSSNPLT |
| NO: 78 | (SEQ ID NO: 53) | (SEQ ID NO: 44) | (SEQ ID NO: 35) |
| SEQ ID | RASKNIYTYLA | NARTLAG | QHHYDTPYT |
| NO: 79 | (SEQ ID NO: 54) | (SEQ ID NO: 45) | (SEQ ID NO: 36) |
| SEQ ID | RASKNVYSYLA | NAKTLAE | QHHYNIPFT |
| NO: 80 | (SEQ ID NO: 55) | (SEQ ID NO: 46) | (SEQ ID NO: 37) |
| SEQ ID | RASKSVSTSGYSYMH | LVSNLES | QHIRELTT |
| NO: 81 | (SEQ ID NO: 56) | (SEQ ID NO: 47) | (SEQ ID NO: 38) |
| SEQ ID | RANSGISFIN | GTANLAS | QQWSSNPLT |
| NO: 82 | (SEQ ID NO: 57), | (SEQ ID NO: 48) | (SEQ ID NO: 39) |
| | RAQSGISFIN | | |
| | (SEQ ID NO: 58) | | |
| SEQ ID | RSSQS LVHSNGNTYLH, | KVSNRFS | SQSTHIPLA |
| NO: 83 | (SEQ ID NO: 59) | (SEQ ID NO: 49) | (SEQ ID NO: 40) |
| | RSSQSLVHSSGNTYLH | | |
| | (SEQ ID NO: 60), | | |
| | RSSQSLVHSTGNTYLH | | |
| | (SEQ ID NO: 61), | | |
| | RSSQSLVHSQGNTYLH | | |
| | (SEQ ID NO: 62) | | |

In some instances, the TM4SF1 antigen binding domain comprises a heavy chain immunoglobulin variable region comprising a complementarity determining region 1 (CDR1) comprising the sequence of SEQ ID NO:23, 24, 25, 26, 27, 28, 29, 30, 31; a CDR2 comprising the sequence of SEQ ID NO:13, 14, 15, 16, 17, 18, 19, 20, 21, 22; and a CDR3 comprising the sequence of SEQ ID NO:1, 2, 3, 4, 5, 6, 7, 8, 9, 10, 11, or 12 and a light chain immunoglobulin variable region comprising a CDR1 comprising the sequence of SEQ ID NO:50, 51, 52, 53, 54, 55, 56, 57, 58, 59, 60, 61, 62,; a CDR2 comprising the sequence of SEQ ID NO:41, 42, 43, 44, 45, 46, 47, 48, 49; and a CDR3 comprising the sequence of SEQ ID NO:32, 33, 34, 35, 36, 37, 38, 39, 40.

In some aspects, TM4SF1 antigen binding domain comprises any of the variable heavy, variable light, or CDRs from U.S. Pat. No. 11,208,495, incorporated by reference in its entirety herein for its teaching of variable heavy, variable light, and CDRs of a TM4SF1 antigen binding domain.

### 2. Transmembrane Domain

In some instances, the transmembrane domain comprises an immunoglobulin Fc domain. In some instances, the immunoglobulin Fc domain can be an immunoglobulin G Fc domain.

In some aspects, the transmembrane domain comprises a transmembrane domain of a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain. In some instances, the transmembrane domain comprises a CD8α domain, CD3ζ, FcεR1γ, CD4, CD7, CD28, OX40, or H2-Kb.

In some instances, the transmembrane domain can be located between the TM4SF1 antigen binding domain and the intracellular signaling domain.

### 3. Intracellular Signaling Domain

In some instances, the intracellular signaling domain can be a T cell signaling domain. For example, the intracellular signaling domain can comprise a CD3ζ signaling domain. In some instances, CD3ζ signaling domain is the intracellular domain of CD3ζ.

In some instances, the intracellular signaling domain comprises a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain comprises a CD3ζ signaling domain and a co-stimulatory signaling region, wherein the co-stimulatory signaling region comprises the cytoplasmic domain of CD28, 4-1BB, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### 4. Hinge Region

Any of the disclosed CAR polypeptides can further comprise a hinge region. For example, disclosed are CAR polypeptides comprising a TM4SF1 antigen binding domain, a transmembrane domain, and an intracellular signaling domain and further comprising a hinge region.

In some instances, the hinge region can be located between the TM4SF1 antigen binding domain and the transmembrane domain.

In some instances, the hinge region allows for the TM4SF1 antigen binding domain to bind to the antigen. For example, the hinge region can increase the distance of the binding domain to the cell surface and provide flexibility.

In some aspects, the hinge region is from CD3zeta, CD4, CD8, CD28, or heavy chain of immunoglobulin.

### 5. Tag

In some instances, any of the disclosed CAR polypeptides can further comprise a tag. In some instances, the tag can be located between the TM4SF1 antigen binding domain and the transmembrane domain or between the TM4SF1 antigen binding domain and a hinge region. In some instances, the tag can be a hemagglutinin tag, histidine tag, glutathione-S-transferase tag, or fluorescent tag. For example, the tag can be any sequence/molecule/compound capable of aiding in the purification of the CAR polypeptide or capable of detecting the CAR polypeptide.

### C. Nucleic Acid Sequences

Disclosed are nucleic acid sequences capable of encoding any of the disclosed CAR polypeptides. For example, disclosed are nucleic acid sequences capable of encoding a CAR polypeptide comprising a TM4SF1 antigen binding domain, a transmembrane domain, and an intracellular signaling domain.

In some aspects, the disclosed nucleic acid sequences can be DNA or RNA. sequences.

In some aspects, the full vector sequence comprising VHVL or VLVH are shown in SEQ ID NOS:128 and 129

In some instances, the TM4SF1 antigen binding domain comprises an amino acid sequence set forth in SEQ ID NO: **. In some instances, the TM4SF1 antigen binding domain can comprise a heavy chain variable region, a light chain variable region, and a linker that links the heavy chain variable region to the light chain variable region. For example, SEQ ID NOs: ** comprise the heavy chain variable region, linker, and light chain variable region (see Table ***). In some instances, the linker can be directly involved in the binding of TM4SF1 to the TM4SF1 antigen binding domain. In some instances, the linker can be indirectly involved in the binding of TM4SF1 to the TM4SF1 antigen binding domain.

### 1. TM4SF1 Antigen Binding Domain

In some instances, the nucleic acid sequence that encodes the TM4SF1 antigen binding domain comprises one or more of the nucleic acid sequences described in U.S. Pat. No. 11,208,495, hereby incorporated by reference in its entirety.

In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some instances, the TM4SF1 antigen binding domain comprises a variable heavy chain comprising a sequence having at least 90% identity to a sequence set forth in SEQ ID NOs:106, 107, 108, 109, 110, 111, 112, 113, or 114 (See Table 8). In some instances, the TM4SF1 antigen binding domain comprises a variable heavy chain comprising a sequence having at least 90, 91, 92, 93, 94, 95, 96, 97, 98, 99, or 100% identity to a sequence set forth in SEQ ID NOs: 106, 107, 108, 109, 110, 111, 112, 113, or 114.

TABLE 8
Examples of heavy chain sequences of TM4SF1
antigen binding domains

| | TM4SF1 Heavy Chain nucleic |
|---|---|
| SEQ ID NO: | acid sequence |
| SEQ ID NO: 106 | CAGATCCAGTTGGTGCAGTCTGGACCTGAG |
| | CTGAAGAAGCCTGGAGAGACAGTCAAGAT |
| | CTCCTGCAAGGCTTCTGGGTATTCCTTCAG |
| | AGACTATGGAATGAACTGGGTGAAGCAGG |
| | CTCCAGGAAGGACTTTTAAGTGGATGGGCT |
| | GGATAAACACCTACACTGGAGCGCCAGTA |
| | TATGCTGCTGACTTCAAGGGACGGTTTGCC |
| | TTCTCTTTGGACACCTCTGCCAGCGCTGCC |
| | TTTTTGCAGATCAACAACCTCAAAAATGAA |
| | GACACGGCTACATATTTCTGTGCAAGATGG |
| | GTCTCCTACGGTAATAACCGCAACTGGTTC |
| | TTCGATTTTTGGGGCGCAGGGACCACGGTC |
| | ACCGTCTCCTCA |
| SEQ ID NO: 107 | GAGGTCCAGCTGCAACAGTCTGGACCTGA |
| | GCTGGTGAAGCCTGGGGCTTCAGTGAAGA |
| | TATCCTGCAAGACTTCTGGATACACATTCA |
| | CTGATTACACCATGCACTGGGTGAGGCAG |
| | AGCCATGGAAAGAGCCTTGAGTGGATTGG |
| | AAGTTTTAATCCTAACAATGGTGGTCTTAC |
| | TAACTACAACCAGAAGTTCAAGGGCAAGG |
| | CCACATTGACTGTGGACAAGTCTTCCAGCA |
| | CAGTGTACATGGACCTCCGCAGCCTGACAT |
| | CTGAGGATTCTGCAGTCTATTACTGTACAA |
| | GAATCCGGGCTACGGGCTTTGACTCCTGGG |
| | GCCAGGGCACCACTCTCACAGTCTCCTCA |
| SEQ ID NO: 108 | GAGGTCCAGGTACAGCAGTCTGGACCTGA |
| | ACTGGTAAAGCCTGGGGCTTCAGTGAAGA |
| | TGTCCTGTAAGGCTTCTGGATACACATTCA |
| | CTAGCTATGTCATGCACTGGGTGAAGCAG |
| | AAGCCTGGGCAGGGCCTTGAGTGGATTGG |
| | ATATATTAATCCTAACAATGATAATATTAA |
| | CTACAATGAGAAGTTCAAAGGCAAGGCCT |
| | CACTGACTTCAGACAAATCCTCCAACACAG |
| | TCTACATGGAGCTCAGCAGCCTGACCTCTG |
| | AGGACTCTGCGGTCTATTACTGTGCAGGCT |
| | ATGGTAACTCCGGAGCTAACTGGGGCCAA |
| | GGGACTCTGGTCACTGTCTCTGCA |
| SEQ ID NO: 109 | CAGATCCAGTTGGTGCAGTCTGGACCTGAG |
| | CTGAAGAAGCCTGGAGAGACAGTCAAGAT |
| | CTCCTGCAAGGCTTCTGGGTATACCTTCAC |
| | AAACTATGGAGTGAAGTGGGTGAAGCAGG |
| | CTCCAGGAAAGGATTTAAAGTGGATGGGC |
| | TGGATAAACACCTACACTGGAAATCCAATT |
| | TATGCTGCTGACTTCAAGGGACGGTTTGCC |
| | TTCTCTTTGGAGACCTCTGCCAGCACTGCC |
| | TTTTTGCAGATCAACAACCTCAAAAATGAG |
| | GACACGGCTACATATTTCTGTGTAAGATTC |
| | CAATATGGCGATTACCGGTACTTCGATGTC |
| | TGGGGCGCAGGGACCACGGTCACCGTCTC |
| | CTCA |
| SEQ ID NO: 110 | GAGGTCCAGCTGCAGCAGTCTGGACCTGA |
| | GCTGGTAAAGCCTGGGGCTTCAGTGAAGC |
| | TGTCCTGCAAGGCTTCTGGATACACAGTCA |
| | CTAGCTATGTTATGCACTGGGTGAAGCAGA |
| | AGCCTGGGCAGGGCCTTGAGTGGATTGGA |
| | TATATTAATCCTTACAGTGATGTTACTAAC |
| | TGCAATGAGAAGTTCAAAGGCAAGGCCAC |
| | ACTGACTTCAGACAAAACCTCCAGCACAG |
| | CCTACATGGAGCTCAGCAGCCTGACCTCTG |
| | AGGACTCTGCGGTCTATTACTGTTCCTCCT |
| | ACGGTGGGGGGTTTGCTTACTGGGGCCAA |
| | GGGACTCTGGTCACTGTCTCTGCA |
| SEQ ID NO: 130 | GAGGTCCAGCTGCAGCAGTCTGGACCTGA |
| | GCTGGTAAAGCCTGGGGCTTCAGTGAAGA |
| | TGTCCTGCAAGGCTTCTGGATACACATTCT |
| | CTAGCTATGTTATGCACTGGGTGAAGCAGA |
| | AGCCTGGGCAGGGCCTTGAGTGGATTGGA |
| | TATATTAATCCTTACAGTGATGTCACTAAC |
| | TACAATGAGAAGTTCAAAGGCAAGGCCAC |
| | ACTGACTTCAGACAGATCCTCCAACACAGC |
| | CTACATGGAACTCAGCAGCCTGACCTCTGA |
| | GGACTCTGCGGTCTATTACTGTGCAAGAAA |
| | TTACTTCGACTGGGGCCGAGGGACTCTGGT |
| | CACAGTCTCTGCA |
| SEQ ID NO: 111 | CAGATCCAGTTGGTGCAGTCTGGACCTGAG |
| | CTGAAGAAGCCTGGAGAGACAGTCAAGAT |
| | CTCCTGCAAGGCTTCTGGGTTTACCTTCAC |
| | AAACTATCCAATGCACTGGGTGAAGCAGG |
| | CTCCAGGAAAGGGTTTAAAGTGGATGGGC |
| | TGGATAAACACCTACTCTGGAGTGCCAAC |
| | ATATGCAGATGACTTCAAGGGACGGTTTGC |
| | CTTCTCTTTGGAAACCTCTGCCAGCACTGC |
| | ATATTTGCAGATCAACAACCTCAAAAATG |
| | AGGACATGGCTACATATTTCTGTGCAAGAG |
| | GGGGCTACGATGGTAGCAGGGAGTTTGCT |
| | TACTGGGGCCAAGGGACTCTGGTCACTGTC |
| | TCT |
| SEQ ID NO: 112 | TCTACCGGACAGGTGCAGTTGGTTCAGTCT |
| | GGCGCCGAAGTGAAGAAACCTGGCGCTTC |
| | TGTGAAGGTGTCCTGCAAGGCCTCTGGCTA |
| | CACCTTTACCAACTACGGCGTGAAATGGGT |
| | CCGACAGGCTCCTGGACAGGATCTGGAAT |
| | GGATGGGCTGGATCAACACCTACACCGGC |
| | AATCCTATCTACGCCGCCGACTTCAAGGGC |
| | AGAGTGACCATGACCACCGACACCTCTAC |
| | CTCCACCGCCTTCATGGAACTGCGGTCCCT |
| | GAGATCTGACGACACCGCCGTGTACTACTG |
| | CGTGCGGTTTCAGTACGGCGACTACCGGTA |
| | CTTTGATGTGTGGGGCCAGGGCACACTGGT |
| | CACCGTTTCTTCCGCTTCTACCAAGGGACC |
| | CAGCGTGTTCCCTCTGGCTCCTTCCTCTAA |
| | ATCCACCTCTGGCGGAACCGCTGCTCTGGG |
| | CTGTCTGGTCAAGGATTACTTCCCTGAGCC |
| | TGTGACCGTGTCCTGGAACTCTGGTGCTCT |
| | GACATCCGGCGTGCACACCTTTCCAGCTGT |
| | GCTGCAGTCCTCTGGCCTGTACTCTCTGTC |
| | CTCTGTCGTGACCGTGCCTTCTAGCTCTCT |
| | GGGCACCCAGACCTACATCTGCAACGTGA |
| | ACCACAAGCCTTCCAACACCAAGGTGGAC |
| | AAGAAGGTGGAACCCAAGTCCTGCGACAA |
| | GACCCACACCTGTCCTCCATGTCCTGCTCC |
| | AGAAGCTGCTGGCGCTCCCTCTGTGTTCCT |
| | GTTTCCTCCAAAGCCTAAGGACACCCTGAT |
| | GATCTCTCGGACCCCTGAAGTGACCTGCGT |
| | GGTGGTGGATGTGTCTCACGAGGACCCAG |
| | AAGTGAAGTTCAATTGGTACGTGGACGGC |
| | GTGGAAGTGCACAACGCCAAGACCAAGCC |
| | TAGAGAGGAACAGTACAACTCCACCTACA |
| | GAGTGGTGTCCGTGCTGACCGTGCTGCACC |
| | AGGATTGGCTGAACGGCAAAGAGTACAAG |
| | TGCAAGGTGTCCAACAAGGCACTGCCCGC |
| | TCCTATCGAAAAGACCATCTCCAAGGCTAA |
| | GGGCCAGCCTCGGGAACCTCAGGTTTACA |
| | CCCTGCCTCCATCTCGGGAAGAGATGACCA |
| | AGAACCAGGTGTCCCTGACCTGCCTCGTGA |
| | AGGGCTTCTACCCTTCCGATATCGCCGTGG |
| | AATGGGAGTCCAATGGCCAGCCTGAGAAC |
| | AACTACAAGACAACCCCTCCTGTGCTGGAC |
| | TCCGACGGCTCATTCTTCCTGTACTCCAAG |
| | CTGACAGTGGACAAGTCTCGGTGGCAGCA |
| | GGGCAACGTGTTCTCCTGTTCTGTGATGCA |
| | CGAGGCCCTGCACAACCACTACACACAGA |
| | AGTCCCTGTCTCTGTCCCCTGGCAAGTGA |
| SEQ ID NO: 113 | GAAGTGCAGTTGGTGCAGTCTGGCGCCGA |
| | AGTGAAGAAACCTGGCGCTTCTGTGAAGG |
| | TGTCCTGCAAGGCCTCTGGCTACACCTTTA |
| | CCAACTACGGCGTGAAATGGGTCCGACAG |
| | GCTCCTGGACAAGGCCTGGAATGGATGGG |
| | CTGGATCAACACCTACACCGGCAATCCTAT |
| | CTACGCCGCCGACTTCAAGGGCAGAGTGA |
| | CCATGACCACCGACACCTCTACCTCCACCG |
| | CCTACATGGAACTGCGGTCCCTGAGATCTG |
| | ACGACACCGCCGTGTACTACTGCGTGCGGT |
| | TTCAGTACGGCGACTACCGGTACTTTGATG |
| | TGTGGGGCCAGGGCACACTGGTCACCGTTT |
| | CTTCCGCTTCTACCAAGGGACCCAGCGTGT |
| | TCCCTCTGGCTCCTTCCTCTAAATCCACCTC |
| | TGGCGGAACCGCTGCTCTGGGCTGTCTGGT |
| | CAAGGATTACTTCCCTGAGCCTGTGACCGT |
| | GTCCTGGAATTCTGGTGCTCTGACATCCGG |
| | CGTGCACACCTTTCCAGCTGTGCTGCAGTC |
| | CTCTGGCCTGTACTCTCTGTCCTCTGTCGTG |
| | ACCGTGCCTTCTAGCTCTCTGGGCACCCAG |
| | ACCTACATCTGCAACGTGAACCACAAGCCT |
| | TCCAACACCAAGGTGGACAAGAAGGTGGA |
| | ACCCAAGTCCTGCGACAAGACCCACACCT |
| | GTCCTCCATGTCCTGCTCCAGAAGCTGCTG |
| | GCGCTCCCTCTGTGTTCCTGTTTCCTCCAAA |
| | GCCTAAGGACACCCTGATGATCTCTCGGAC |
| | CCCTGAAGTGACCTGCGTGGTGGTGGATGT |
| | GTCTCACGAGGACCCAGAAGTGAAGTTCA |
| | ATTGGTACGTGGACGGCGTGGAAGTGCAC |
| | AACGCCAAGACCAAGCCTAGAGAGGAACA |
| | GTACAACTCCACCTACAGAGTGGTGTCCGT |
| | GCTGACCGTGCTGCACCAGGATTGGCTGA |
| | ACGGCAAAGAGTACAAGTGCAAGGTGTCC |
| | AACAAGGCACTGCCCGCTCCTATCGAAAA |
| | GACCATCTCCAAGGCTAAGGGCCAGCCTC |
| | GGGAACCTCAGGTTTACACCCTGCCTCCAT |
| | CTCGGGAAGAGATGACCAAGAACCAGGTG |
| | TCCCTGACCTGCCTCGTGAAGGGCTTCTAC |
| | CCTTCCGATATCGCCGTGGAATGGGAGTCC |
| | AATGGCCAGCCTGAGAACAACTACAAGAC |
| | AACCCCTCCTGTGCTGGACTCCGACGGCTC |
| | ATTCTTCCTGTACTCCAAGCTGACAGTGGA |
| | CAAGTCTCGGTGGCAGCAGGGCAACGTGT |
| | TCTCCTGTTCTGTGATGCACGAGGCCCTGC |
| | ACAACCACTACACACAGAAGTCCCTGTCTC |
| | TGTCCCCTGGCAAGTGA |
| SEQ ID NO: 114 | GAGGTGCAGCTGGTTGAATCTGGCGGAGG |
| | ACTTGTGAAGCCTGGCGGCTCTCTGAGACT |
| | GTCTTGTGCCGCCTCTGGCTTCACCTTCTCC |
| | AGCTTTGCCATGTCCTGGGTCCGACAGGCT |
| | CCTGGCAAAGGACTGGAATGGGTGTCCAC |
| | CATCTCCTCCGGCTCCATCTACATCTACTA |
| | CACCGACGGCGTGAAGGGCAGATTCACCA |
| | TCAGCAGAGACAACGCCAAGAACTCCCTG |
| | TACCTGCAGATGAACAGCCTGAGAGCCGA |
| | GGACACCGCCGTGTACTATTGTGCCAGACG |
| | GGGCATCTACTATGGCTACGACGGCTACGC |
| | TATGGACTATTGGGGACAGGGCACACTGG |
| | TCACCGTGTCCTCTGCTTCTACCAAGGGAC |
| | CCAGCGTGTTCCCTCTGGCTCCTTCCTCTA |
| | AATCCACCTCTGGCGGAACCGCTGCTCTGG |
| | GCTGTCTGGTCAAGGATTACTTCCCTGAGC |
| | CTGTGACCGTGTCCTGGAACTCTGGTGCTC |
| | TGACATCCGGCGTGCACACCTTTCCAGCTG |
| | TGCTGCAGTCCTCTGGCCTGTACTCTCTGT |
| | CCTCTGTCGTGACCGTGCCTTCTAGCTCTCT |
| | GGGCACCCAGACCTACATCTGCAACGTGA |
| | ACCACAAGCCTTCCAACACCAAGGTGGAC |
| | AAGAAGGTGGAACCCAAGTCCTGCGACAA |
| | GACCCACACCTGTCCTCCATGTCCTGCTCC |
| | AGAAGCTGCTGGCGCTCCCTCTGTGTTCCT |
| | GTTTCCTCCAAAGCCTAAGGACACCCTGAT |
| | GATCTCTCGGACCCCTGAAGTGACCTGCGT |
| | GGTGGTGGATGTGTCTCACGAGGACCCAG |
| | AAGTGAAGTTCAATTGGTACGTGGACGGC |
| | GTGGAAGTGCACAACGCCAAGACCAAGCC |
| | TAGAGAGGAACAGTACAACTCCACCTACA |
| | GAGTGGTGTCCGTGCTGACCGTGCTGCACC |
| | AGGATTGGCTGAACGGCAAAGAGTACAAG |
| | TGCAAGGTGTCCAACAAGGCACTGCCCGC |
| | TCCTATCGAAAAGACCATCTCCAAGGCTAA |
| | GGGCCAGCCTCGGGAACCTCAGGTTTACA |
| | CCCTGCCTCCATCTCGGGAAGAGATGACCA |
| | AGAACCAGGTGTCCCTGACCTGCCTCGTGA |
| | AGGGCTTCTACCCTTCCGATATCGCCGTGG |
| | AATGGGAGTCCAATGGCCAGCCTGAGAAC |
| | AACTACAAGACAACCCCTCCTGTGCTGGAC |
| | TCCGACGGCTCATTCTTCCTGTACTCCAAG |
| | CTGACAGTGGACAAGTCTCGGTGGCAGCA |
| | GGGCAACGTGTTCTCCTGTTCTGTGATGCA |
| | CGAGGCCCTGCACAACCACTACACACAGA |
| | AGTCCCTGTCTCTGTCCCCTGGCAAGTGA |

In some instances, the TM4SF1 antigen binding domain comprises a variable light chain comprising a sequence having at least 90% identity to a sequence set forth in SEQ ID NOs:115, 116, 117, 118, 119, 120, 121, 122, 123, 124, 125, 126, or 127 (See Table 9). In some instances, the TM4SF1 antigen binding domain comprises a variable light chain comprising a sequence having at least 90, 91, 92, 93, 94, 95, 96, 97, 98, 99, or 100% identity to a sequence set forth in SEQ ID NOs: 115, 116, 117, 118, 119, 120, 121, 122, 123, 124, 125, 126, or 127.

TABLE 9
Examples of light chain sequences of TM4SF1
antigen binding domains

| | TM4SF1 nucleic acid light |
|---|---|
| SEQ ID NO: | chain sequences |
| SEQ ID NO: 115 | GATGTTTTGATGACCCAAACTCCACTCTCC |
| | CTGCCTGTCCGTCTTGGAGATCAGGCCTCC |
| | ATCTCTTGTAGATCTAGTCAGACCCTTGTA |
| | CATAGTAATGGAAACACCTATTTAGAATG |
| | GTACCTGCAGAAACCAGGCCAGTCTCCAA |
| | AACTCTTGATCTACAAAGTTTCCAATCGAC |
| | TTTCTGGGGTCCCAGACAGGTTCAGTGGCA |
| | GTGGATCAGGGACAGATTTCACACTCAAG |
| | ATCAGCAGAGTGGAGACTGAGGATCTGGG |
| | AGTTTATTACTGCTTTCAAGGTTCACATGG |
| | TCCGTGGACGTTCGGTGGAGGCACCAAGC |
| | TGGAAATCAAA |
| SEQ ID NO: 116 | GACATTGTGATGTCACAGTCTCCATCCTCC |
| | CTGGCTGTGTCAGCAGGAGAGAAGGTCAC |
| | TATGAGCTGCAAATCCAGTCAGAGTCTGCT |
| | CAACAGTAGAACCCGAAAGAACTACTTGG |
| | CTTGGTACCAGCAGAAACCAGGGCAGTCT |
| | CCTAAACTGCTGATCTACTGGGCATCCACT |
| | AGGGAATCTGGGGTCCCTGATCGCTTCACA |
| | GGCAGTGGATCTGGGACAGATTTCACTCTC |
| | ACCATCAGCAATGTGCAGGCTGAAGACCT |
| | GACAGTTTATTACTGCAAGCAATCTTATAA |
| | TCCTCCGTGGACGTTCGGTGGAGGCACCAA |
| | GCTGGAAATCAAA |
| SEQ ID NO: 117 | GACATCCAGATGACTCAGTCTCCAGCCTCC |
| | CTATCTGCATCTGTGGGAGAAACTGTCACC |
| | ATCACATGTCGAACAAGTAAAAATATTTTC |
| | AATTTTTTAGCATGGTATCACCAGAAACAG |
| | GGAAGATCTCCTCGACTCCTGGTCTCTCAT |
| | ACAAAAACCTTAGCAGCAGGTGTGCCATC |
| | AAGGTTCAGTGGCAGTGGCTCAGGCACAC |
| | AGTTTTCTCTGAAGATCAACAGCCTGCAGC |
| | CTGAAGATTTTGGGATTTATTACTGTCAAC |
| | ATCATTATGGTACTCCGTGGACGTTCGGTG |
| | GAGGCACCAAACTGGAAATCAAA |
| SEQ ID NO: 118 | CAAATTATTCTCTCCCAGTCTCCAGCAATC |
| | CTGTCTGCATCTCCAGGGGAGAAGGTCAC |
| | GATGACTTGCAGGGCCAACTCAGGTATTA |
| | GTTTCATCAACTGGTACCAGCAGAAGCCA |
| | GGATCCTCCCCCAAACCCTGGATTTATGGC |
| | ACAGCCAACCTGGCTTCTGGAGTCCCTGCT |
| | CGCTTCGGTGGCAGTGGGTCTGGGACTTCT |
| | TACTCTCTCACAATCAGCAGAGTGGAGGCT |
| | GAAGACGCTGCCACTTATTACTGCCAGCAG |
| | TGGAGTAGTAACCCGCTCACGTTCGGTGCT |
| | GGGACCAAGCTGGAGTTGAGA |
| SEQ ID NO: 119 | GACATCCAGATGACTCAGTCTCCAGCCTCC |
| | CTATCTGCATCTGTGGGAGAACCTGTCACC |
| | ATCACATGTCGAGCAAGTAAGAATATTTAC |
| | ACATATTTAGCATGGTATCACCAGAAACA |
| | GGGAAAATCTCCTCAGTTCCTGGTCTATAA |
| | TGCAAGAACCTTAGCAGGAGGTGTGCCAT |
| | CAAGGCTCAGTGGCAGTGGATCAGTCACG |
| | CAGTTTTCTCTAAACATCAACACCTTGCAT |
| | CGAGAAGATTTAGGGACTTACTTCTGTCAA |
| | CATCATTATGATACTCCGTACACGTTCGGA |
| | GGGGGGACCAACCTGGAAATAAAA |
| SEQ ID NO: 120 | GACATCCAGATGACTCAGTCTCCAGCCTCC |
| | CTATCTGCATCTGTGGGAGAAACTGTCACC |
| | ATCACATGTCGAGCAAGTAAAAATGTTTAC |
| | AGTTATTTAGCATGGTTTCAACAGAAACAG |
| | GGGAAATCTCCTCAGCTCCTGGTCTATAAT |
| | GCTAAAACCTTAGCAGAAGGTGTGCCATC |
| | AAGGTTCAGTGGCGGGGGATCAGGCACAC |
| | AGTTTTCTCTGAAGATCAACAGCCTGCAGC |
| | CTGCAGATTTTGGGAGTTATTACTGTCAAC |
| | ATCATTATAATATTCCATTCACGTTCGGCT |
| | CGGGGACAAAGTTGGAAATAAAA |
| SEQ ID NO: 121 | GACATTGTGCTGACACAGTCTCCTGCTTCC |
| | TTAGCTGCATCTCTGGGGCAGAGGGCCACC |
| | ACCTCATACAGGGCCAGCAAAAGTGTCAG |
| | TACATCTGGCTATAGTTATATGCACTGGAA |
| | CCAACAGAAACCAGGACAGCCACCCAGAC |
| | TCCTCATCTATCTTGTATCCAACCTAGAAT |
| | CTGGGGTCCCTGCCAGGTTCAGTGGCAGTG |
| | GGTCTGGGACAGACTTCACCCTCAACATCC |
| | ATCCTGTGGAGGAGGAGGATGCTGCAACC |
| | TATTACTGTCAGCACATTAGGGAGCTTACC |
| | ACGTTCGGAGGGGGGACCAAGCTGGAAAT |
| | AAAA |
| SEQ ID NO: 122 | AAGCTTGCCACCATGGAAACCGACACACT |
| | GCTGCTGTGGGTGCTGTTGTTGTGGGTGCC |
| | AGGATCTACCGGAGAGATCATCCTGACAC |
| | AGAGCCCCGCCACATTGTCTCTGAGTCCTG |
| | GCGAGAGAGCTACCCTGTCCTGTAGAGCC |
| | AACTCCGGCATCTCCTTCATCAACTGGTAT |
| | CAGCAGAAGCCCGGCCAGGCTCCTAGACT |
| | GCTGATCTATGGCACCGCTAACCTGGCCTC |
| | TGGCATCCCTGCTAGATTTGGCGGCTCTGG |
| | CTCTGGCAGAGACTTCACCCTGACCATCTC |
| | TAGCCTGGAACCTGAGGACTTCGCCGTGTA |
| | CTACTGCCAGCAGTGGTCTAGCAACCCTCT |
| | GACCTTTGGCGGAGGCACCAAGGTGGAAA |
| | TCAAGAGAACCGTGGCCGCTCCTTCCGTGT |
| | TCATCTTCCCACCATCTGACGAGCAGCTGA |
| | AGTCTGGCACAGCCTCTGTCGTGTGCCTGC |
| | TGAACAACTTCTACCCTCGGGAAGCCAAG |
| | GTGCAGTGGAAGGTGGACAATGCCCTGCA |
| | GTCCGGCAACTCCCAAGAGTCTGTGACCG |
| | AGCAGGACTCCAAGGACTCTACCTACAGC |
| | CTGTCCTCCACACTGACCCTGTCTAAGGCC |
| | GACTACGAGAAGCACAAGGTGTACGCCTG |
| | TGAAGTGACCCACCAGGGACTGTCTAGCC |
| | CCGTGACCAAGTCTTTCAACCGGGGCGAGT |
| | GCTGA |
| SEQ ID NO: 123 | TCTACAGGCGAGATCGTGCTGACCCAGTCT |
| | CCTGCCACATTGTCTCTGAGTCCTGGCGAG |
| | AGAGCTACCCTGTCCTGTAGAGCCAACTCC |
| | GGCATCTCCTTCATCAACTGGTATCAGCAG |
| | AAGCCCGGCCAGGCTCCTAGACTGCTGATC |
| | TATGGCACCGCTAACCTGGCCTCTGGCATC |
| | CCTGCTAGATTTTCCGGCTCTGGCTCTGGC |
| | AGAGACTTCACCCTGACCATCTCTAGCCTG |
| | GAACCTGAGGACTTCGCCGTGTACTACTGC |
| | CAGCAGTGGTCTAGCAACCCTCTGACCTTT |
| | GGCGGAGGCACCAAGGTGGAAATCAAGAG |
| | AACCGTGGCCGCTCCTTCCGTGTTCATCTT |
| | CCCACCATCTGACGAGCAGCTGAAGTCTG |
| | GCACAGCCTCTGTCGTGTGCCTGCTGAACA |
| | ACTTCTACCCTCGGGAAGCCAAGGTGCAGT |
| | GGAAGGTGGACAATGCCCTGCAGTCCGGC |
| | AACTCCCAAGAGTCTGTGACCGAGCAGGA |
| | CTCCAAGGACTCTACCTACAGCCTGTCCTC |
| | CACACTGACCCTGTCTAAGGCCGACTACGA |
| | GAAGCACAAGGTGTACGCCTGTGAAGTGA |
| | CCCACCAGGGACTGTCTAGCCCCGTGACCA |
| | AGTCTTTCAACCGGGGCGAGTGCTGA |
| SEQ ID NO: 124 | TCTACAGGCGAGATCGTGCTGACCCAGTCT |
| | CCTGCCACATTGTCTCTGAGTCCTGGCGAG |
| | AGAGCTACCCTGTCTTGTAGAGCCCAGTCC |
| | GGCATCTCCTTCATCAACTGGTATCAGCAG |
| | AAGCCCGGCCAGGCTCCTAGACTGCTGATC |
| | TATGGCACCGCTAACCTGGCCTCTGGCATC |
| | CCTGCTAGATTTTCCGGCTCTGGCTCTGGC |
| | AGAGACTTCACCCTGACCATCTCTAGCCTG |
| | GAACCTGAGGACTTCGCCGTGTACTACTGC |
| | CAGCAGTGGTCTAGCAACCCTCTGACCTTT |
| | GGCGGAGGCACCAAGGTGGAAATCAAGAG |
| | AACCGTGGCCGCTCCTTCCGTGTTCATCTT |
| | CCCACCATCTGACGAGCAGCTGAAGTCTG |
| | GCACAGCCTCTGTCGTGTGCCTGCTGAACA |
| | ACTTCTACCCTCGGGAAGCCAAGGTGCAGT |
| | GGAAGGTGGACAATGCCCTGCAGTCTGGC |
| | AACTCCCAAGAGTCTGTGACCGAGCAGGA |
| | CTCCAAGGACTCTACCTACAGCCTGTCCTC |
| | CACACTGACCCTGTCTAAGGCCGACTACGA |
| | GAAGCACAAGGTGTACGCCTGTGAAGTGA |
| | CCCACCAGGGACTGTCTAGCCCCGTGACCA |
| | AGTCTTTCAACCGGGGCGAGTGCTGA |
| SEQ ID NO: 125 | TCTACAGGCGAGATCGTGCTGACCCAGTCT |
| | CCTGCCACATTGTCTCTGAGTCCTGGCGAG |
| | AGAGCTACCCTGTCCTGTAGAGCCAACTCC |
| | GGCATCTCCTTCATCAACTGGTATCAGCAG |
| | AAGCCCGGCCAGGCTCCTAGACTGCTGATC |
| | TATGGCACCGCTAACCTGGCCTCTGGCATC |
| | CCTGCTAGATTTTCCGGCTCTGGCTCTGGC |
| | AGAGACTTCACCCTGACCATCTCTAGCCTG |
| | GAACCTGAGGACTTCGCCGTGTACTACTGC |
| | CAGCAGTACAGCAGCAACCCTCTGACCTTT |
| | GGCGGAGGCACCAAGGTGGAAATCAAGAG |
| | AACCGTGGCCGCTCCTTCCGTGTTCATCTT |
| | CCCACCATCTGACGAGCAGCTGAAGTCTG |
| | GCACAGCCTCTGTCGTGTGCCTGCTGAACA |
| | ACTTCTACCCTCGGGAAGCCAAGGTGCAGT |
| | GGAAGGTGGACAATGCCCTGCAGTCCGGC |
| | AACTCCCAAGAGTCTGTGACCGAGCAGGA |
| | CTCCAAGGACTCTACCTACAGCCTGTCCTC |
| | CACACTGACCCTGTCTAAGGCCGACTACGA |
| | GAAGCACAAGGTGTACGCCTGTGAAGTGA |
| | CCCACCAGGGACTGTCTAGCCCCGTGACCA |
| | AGTCTTTCAACCGGGGCGAGTGCTGA |
| SEQ ID NO: 126 | TCTACAGGCGAGATCGTGCTGACCCAGTCT |
| | CCTGCCACATTGTCTCTGAGTCCTGGCGAG |
| | AGAGCTACCCTGTCTTGTAGAGCCCAGTCC |
| | GGCATCTCCTTCATCAACTGGTATCAGCAG |
| | AAGCCCGGCCAGGCTCCTAGACTGCTGATC |
| | TATGGCACCGCTAACCTGGCCTCTGGCATC |
| | CCTGCTAGATTTTCCGGCTCTGGCTCTGGC |
| | AGAGACTTCACCCTGACCATCTCTAGCCTG |
| | GAACCTGAGGACTTCGCCGTGTACTACTGC |
| | CAGCAGTACAGCAGCAACCCTCTGACCTTT |
| | GGCGGAGGCACCAAGGTGGAAATCAAGAG |
| | AACCGTGGCCGCTCCTTCCGTGTTCATCTT |
| | CCCACCATCTGACGAGCAGCTGAAGTCTG |
| | GCACAGCCTCTGTCGTGTGCCTGCTGAACA |
| | ACTTCTACCCTCGGGAAGCCAAGGTGCAGT |
| | GGAAGGTGGACAATGCCCTGCAGTCTGGC |
| | AACTCCCAAGAGTCTGTGACCGAGCAGGA |
| | CTCCAAGGACTCTACCTACAGCCTGTCCTC |
| | CACACTGACCCTGTCTAAGGCCGACTACGA |
| | GAAGCACAAGGTGTACGCCTGTGAAGTGA |
| | CCCACCAGGGACTGTCTAGCCCCGTGACCA |
| | AGTCTTTCAACCGGGGCGAGTGCTGA |
| SEQ ID NO: 127 | GCCATCGTGTTGACCCAGTCTCCAGGCACA |
| | TTGTCTCTGAGCCCTGGCGAGAGAGCTACC |
| | CTGTCCTGCAGATCTTCTCAGTCCCTGGTG |
| | CACTCCAACGGCAACACCTACCTGCACTGG |
| | TACATGCAGAAGCCCGGACAGGCTCCCAG |
| | AGTGCTGATCTACAAGGTGTCCAACCGGTT |
| | CTCTGGCATCCCCGACAGATTTTCCGGCTC |
| | TGGCTCTGGCACCGACTTCACCCTGACCAT |
| | CTCTAGACTGGAACCCGACGACTTCGCCAT |
| | CTACTACTGCTCCCAGTCCACACACATCCC |
| | TCTGGCTTTTGGCCAGGGCACCAAGCTGGA |
| | AATCAAGAGAACCGTGGCCGCTCCTTCCGT |
| | GTTCATCTTCCCACCATCTGACGAGCAGCT |
| | GAAGTCCGGCACAGCTTCTGTCGTGTGCCT |
| | GCTGAACAACTTCTACCCTCGGGAAGCCA |
| | AGGTGCAGTGGAAGGTGGACAATGCCCTG |
| | CAGTCCGGCAACTCCCAAGAGTCTGTGACC |
| | GAGCAGGACTCCAAGGACTCTACCTACAG |
| | CCTGTCCTCCACACTGACCCTGTCTAAGGC |
| | CGACTACGAGAAGCACAAGGTGTACGCCT |
| | GTGAAGTGACCCACCAGGGCCTGTCTAGC |
| | CCTGTGACCAAGTCTTTCAACCGGGGCGAG |
| | TGTTGA |

In some embodiments are provided nucleic acid sequences that are codon optimized for expression in a host cell, e.g., a bacterium, such as E. coli, or a eukaryotic cell, such as a CHO cell. In some aspects, the nucleic acid sequences are codon optimized for expression in CHO cells. In some examples, a TM4SF1 antigen binding domain of the present disclosure comprises a heavy chain variable domain encoded by a codon optimized nucleic acid sequence as set forth in any one of SEQ ID NOs: 92, 93, 94, 95, 96, 97, or 98. In some examples, an anti-TM4SF1 antibody of the present disclosure comprises a light chain variable domain encoded by a codon optimized nucleic acid sequence as set forth in any one of SEQ ID NOs: 99, 100, 101, 102, 103, 104, or 105. In certain instances, the nucleic acid sequence ofany one of SEQ ID NOs: 92, 93, 94, 95, 96, 97, or 98 is a nucleic acid sequence codon optimized for expression in CHO cell. In certain instances, the nucleic acid sequence of any one of SEQ ID NOs: 99, 100, 101, 102, 103, 104, or 105 is a nucleic acid sequence codon optimized for expression in CHO cell.

TABLE 6
Examples of codon optimized heavy chain sequences of TM4SF1 antigen binding
domains

| SEQ ID NO: | Codon Optimized Sequence |
|---|---|
| SEQ ID NO: 92 | caaattcagt tggttcaatc cggccctgag ctcaagaagc ctggagagac |
| | agtgaagata agttgtaagg ctagtggcta ttcatttcga gattatggga tgaattgggt |
| | caagcaggcc ccagggcgga ccttcaaatg gatggggtgg atcaatactt |
| | acactggcgc accagtatat gcagctgatt ttaagggtcg ctttgcattt tcacttgata |
| | cttcagccag tgccgctttt ttgcaaatca acaatctcaa aaatgaagac actgctacat |
| | atttctgcgc caggtgggtg agctatggca ataacagaaa ttggttcttt gacttttggg |
| | gcgcaggcac caccgtcact gtctcatca |
| SEQ ID NO: 93 | gaggtacaac tgcaacagag tggacctgaa cttgtcaaac ctggagcaag |
| | tgtgaagatt agctgtaaaa ccagtggcta cacatttacc gattatacta tgcactgggt |
| | aagacagagc cacggaaaat cactggagtg gattggtagt ttcaatccta |
| | acaacggagg attgacaaat tacaaccaga agttcaaagg gaaagccacc |
| | ttgacagttg ataagtcctc aagtaccgtg tatatggatc tgcgttctct cacaagtgaa |
| | gatagcgcag tttactactg tacccgcatc cgagccaccg ggttcgattc |
| | atggggtcag gggacaacac tgactgtttc ttct |
| SEQ ID NO: 94 | gaagttcaag ttcagcaaag cgggcctgag cttgtcaagc caggcgcatc |
| | agtcaaaatg agctgtaagg cttccgggta caccttcacc agttatgtca tgcattgggt |
| | aaaacaaaag ccaggacagg gactcgagtg gataggatac attaacccaa |
| | ataacgacaa cattaactac aacgagaaat tcaagggcaa agcatcattg acttccgata |
| | aatcctctaa caccgtgtac atggagctga gttcattgac cagcgaggat tctgccgtgt |
| | actactgtgc aggttatggc aactctggtg ctaactgggg gcaggggact |
| | ctggtcacag tcagcgca |
| SEQ ID NO: 95 | caaatccaac ttgtccagag cggtcccgag ttgaagaagc ctggcgaaac |
| | cgtgaaaatc tcatgcaagg ccagtggata tacatttaca aactatggcg tcaagtgggt |
| | gaaacaagcc ccaggtaaag acttgaaatg gatgggatgg atcaacacat |
| | acacagggaa tcctatctat gcagccgact ttaaaggcag atttgccttc agtttggaga |
| | catctgcctc caccgctttc ctgcaaataa ataacctgaa aaatgaagat accgctacat |
| | acttctgtgt acggttccag tacggagatt accgctattt cgatgtgtgg ggcgcaggta |
| | ccacagtaac cgtctcctca |
| SEQ ID NO: 96 | gaagtccagc ttcagcaatc cggcccagaa ctggtaaaac caggcgcaag |
| | tgttaagttg agttgcaaag ccagtggtta taccgttact tcatacgtca tgcattgggt |
| | aaaacaaaag cccggccaag ggcttgaatg gatcggctac atcaaccctt |
| | actctgacgt caccaactgc aacgagaaat tcaaagggaa agccacattg |
| | acctctgaca agacaagcag taccgcctac atggagcttt ctagtttgac ttctgaagac |
| | tctgctgtct actactgtag cagctacggc ggcggctttg cttactgggg ccagggtaca |
| | ttggtgactg tgagtgca |
| SEQ ID NO: 97 | gaggtacagc ttcagcagag tggtccagaa ctcgtcaagc ctggggcaag |
| | cgttaagatg agttgtaaag catccggtta cacattcagt agctatgtta tgcactgggt |
| | caaacagaag cctgggcagg ggttggagtg gatcggatat ataaatccct |
| | attcagacgt aactaattat aatgaaaagt tcaaggggaa agcaaccttg acaagtgacc |
| | ggtcatctaa taccgcatac atggagctga gctcattgac aagtgaggac tctgctgtgt |
| | attactgtgc ccggaactac ttcgactggg gtaggggcac actggtaact gttagtgca |
| SEQ ID NO: 98 | cagatacaac tcgtccagtc aggtccagag ttgaagaaac ccggagaaac |
| | tgtgaagata tcctgtaaag ccagcggctt tactttcaca aactacccca tgcattgggt |
| | gaagcaggcc ccggaaaag gactcaaatg gatgggatgg atcaacacat |
| | acagtggggt gcctacttac gcagacgatt tcaaaggaag gttcgcattt agcttggaaa |
| | ctagcgcatc tacagcatat ctccagatta acaatcttaa aaatgaggat atggcaacat |
| | acttctgcgc taggggaggt tacgatggga gcagggagtt cgcttattgg |
| | gggcaaggga ctcttgtgac tgtaagt |

TABLE 7
Examples of codon optimized light chain sequences of TM4SF1 antigen binding
domains

| SEQ ID NO: | Codon Optimized Sequence |
|---|---|
| SEQ ID NO: 99 | gacgtactta tgacacaaac tcccttgagc ttgccagtac ggcttggcga |
| | tcaagcttca atttcatgtc gttcttctca aacacttgtc cactcaaatg |
| | ggaatacata tttggaatgg tatctccaaa agcccggcca atccccaaaa |
| | ttgttgattt acaaggtgtc taatcgactc tcaggcgtcc ccgaccgatt |
| | ctccgggagc gggtccggta cagacttcac cttgaaaatc |
| | tccagggtag aaactgaaga cctcggagtc tactattgtt tccaggggtc |
| | acacggcccc tggacatttg gaggaggaac taagctcgag atcaaa |
| SEQ ID NO: 100 | gacatagtta tgtcccagtc tccatccagc ttggctgtca gcgccggaga |
| | gaaagtgact atgagttgta aatcttccca gtccctgctt aactcacgta |
| | ctcggaagaa ttatcttgcc tggtatcaac aaaagccagg tcaaagtcct |
| | aagctcctta tttactgggc ctcaacacgg gagtcaggtg tccccgatcg |
| | cttcacaggt agtgggagtg gtactgactt cactctcacc atttcaaatg |
| | tccaagcaga agacttgact gtgtattact gtaagcagag ttacaaccct |
| | ccttggacct ttggtggggg gaccaaactg gagatcaag |
| SEQ ID NO: 101 | gacattcaga tgacccagtc accagcatct ttgagcgcat ccgttgggga |
| | gactgtgaca atcacatgcc gaaccagtaa gaacatcttc aacttcctcg |
| | catggtacca tcaaaagcag ggcaggtctc ccagactgct tgtctctcac |
| | accaagacac tggcagcagg cgtccccagc cggtttagtg gtagtggatc |
| | tggcacacag tttagtttga aaatcaattc cctgcaaccc gaagacttcg |
| | gcatatacta ttgccagcac cactatggga caccttggac tttcggaggt |
| | ggtactaaac ttgagattaa a |
| SEQ ID NO: 102 | caaataattc tgtcacagtc ccccgctata cttagtgctt caccaggaga |
| | aaaagtgacc atgacttgta gagctaattc tggcatatca ttcatcaact |
| | ggtatcaaca aaagccaggt tcctccccca agccatggat ttacgggacc |
| | gccaaccttg cttctggggt acccgctcgt ttcggcggat caggttcagg |
| | aacttcctat agcctcacta tcagtcgggt tgaagctgag gatgccgcta |
| | catattactg ccagcaatgg tctagtaatc cacttacctt tggagctggc |
| | accaaattgg aacttcgt |
| SEQ ID NO: 103 | gacatccaga tgacacagtc accagcatcc ctgtccgcct cagttgggga |
| | gcctgttacc ataacttgtc gggcaagcaa aaacatatac acctatttgg |
| | cttggtatca ccaaaagcaa ggtaagtcac ctcagtttct tgtatataat |
| | gcccgcacac ttgctggcgg agtaccctct cgattgtctg gatctggcag |
| | cgttacccaa ttcagcctga acatcaacac cctccatcgg gaagatttgg |
| | gtacctattt ctgtcaacat cactacgaca ccccatacac cttcggaggc |
| | ggcacaaatt tggaaattaa a |
| SEQ ID NO: 104 | gacatacaaa tgacacaaag tcccgctagt ctttcagcca gtgttggtga |
| | gactgtgaca ataacctgta gagctagcaa aaatgtctac tcctatctgg |
| | cttggttcca gcagaaacaa ggaaagagtc ctcagttgct cgtatataat |
| | gctaaaactt tggcagaagg cgtcccttct cgtttcagtg gcggaggaag |
| | tgggactcaa ttctcactga agatcaatag cctccagccc gccgactttg |
| | ggagctacta ttgccaacat cattacaaca taccattcac ctttggctca |
| | ggtactaaac tcgaaattaa a |
| SEQ ID NO: 105 | gacatagtgc tcactcagag ccctgcatcc cttgccgcct ccctcggaca |
| | acgagctact acaagctacc gggcatcaaa gtccgttagc acatcaggat |
| | acagctatat gcactggaat cagcaaaagc caggccaacc accccgtctt |
| | ctcatctacc tcgtaagtaa tctggaatca ggcgtgccag cccgattcag |
| | tgggtcaggg tctgggacag atttcaccct caacatccat ccagtagagg |
| | aagaggacgc agcaacatat tactgccaac acattagaga acttaccact |
| | ttcggaggag gaactaaatt ggagatcaaa |

### 2. Transmembrane Domain

In some instances, the transmembrane domain comprises a nucleic acid sequence that encodes an immunoglobulin Fc domain. In some instances, the immunoglobulin Fc domain can be an immunoglobulin G Fc domain.

In some instances, the transmembrane domain comprises a nucleic acid sequence that encodes a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain.

In some instances, the transmembrane domain comprises a nucleic acid sequence that encodes a CD8α domain, CD3ζ, FcεR1γ, CD4, CD7, CD28, OX40, or H2-Kb.

In some instances, the nucleic acid sequence that encodes the transmembrane domain can be located between the nucleic acid sequence that encodes the TM4SF1 antigen binding domain and the nucleic acid sequence that encodes the intracellular signaling domain.

### 3. Intracellular Domain

In some instances, the intracellular signaling domain can be a nucleic acid sequence encoding a T cell signaling domain. For example, the intracellular signaling domain can comprise a nucleic acid sequence that encodes a CD3ζ signaling domain. In some instances, CD3ζ signaling domain is the intracellular domain of CD3ζ.

In some instances, the intracellular signaling domain comprises a nucleic acid that encodes a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain comprises a nucleic acid sequence encoding a CD3ζ signaling domain and a co-stimulatory signaling region, wherein the co-stimulatory signaling region comprises the cytoplasmic domain of CD28, 4-1BB, CD27, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### 4. Hinge Region

In some instances, the hinge region can be a nucleic acid sequence encoding a hinge region. For example, disclosed are nucleic acid sequences that encode the hinge region portion of CD3zeta, CD4, CD8, CD28, or heavy chain of immunoglobulin.

In some instances, the nucleic acid sequence that encodes the hinge region can be located between the nucleic acid sequence that encodes the TM4SF1 antigen binding domain and the nucleic acid sequence that encodes the transmembrane domain.

### D. Vectors

Disclosed are vectors comprising the nucleic acid sequence of the disclosed CAR nucleic acid sequences. In some instances, the vector can be selected from the group consisting of a DNA, a RNA, a plasmid, and a viral vector. In some instances, the vector can comprise a promoter.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be a lentiviral vector. In some aspects, the vector can be a non-viral vector, such as a DNA based vector.

i. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding one or more of the disclosed CAR polypeptides.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Cells

Disclosed are cells comprising any of the CAR polypeptides, CAR nucleic acid sequences, or vectors disclosed herein. These cells can be considered genetically modified.

In some instances, the cell can be a T cell. For example, the T cell can be a CD8+ T cell. In some instances, the cell can be a mammalian cell, such as a human cell.

Thus, disclosed are T cells expressing one of the CAR polypeptides disclosed herein.

In some aspects, the cells can be eukaryotic or prokaryotic. In some aspects, the cells are mammalian cells. In some aspects, the cell is a human cell. In some aspects, the cell is a αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof. In some aspects, the regulatory T cell is a CD8+ T cell or CD4+ T cell.

Disclosed are T cells expressing the one or more of the CAR polypeptides disclosed herein. Disclosed are T cells expressing one or more of the CAR polypeptides disclosed herein that bind human TM4SF1, wherein the T cell has increased specificity to bladder cancer cells.

### F. Compositions

Disclosed are compositions comprising the disclosed CAR polypeptides, nucleic acid sequences, vectors, or cells. Disclosed are compositions comprising a CAR polypeptide comprising a TM4SF1 antigen binding domain, a transmembrane domain, and an intracellular signaling domain. Disclosed are compositions comprising a nucleic acid construct encoding a CAR polypeptide comprising a TM4SF1 antigen binding domain, a transmembrane domain, and an intracellular signaling domain. Also disclosed are compositions comprising a vector comprising a nucleic acid construct, wherein the nucleic acid construct comprises a nucleic acid sequence encoding a CAR polypeptide comprising a TM4SF1 antigen binding domain, a transmembrane domain, and an intracellular signaling domain.

The disclosed compositions can further comprise a pharmaceutically acceptable carrier.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed peptides can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed peptides described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed peptides. That is, a pharmaceutical composition can be provided comprising a therapeutically effective amount of at least one disclosed peptide or at least one product of a disclosed method and a pharmaceutically acceptable carrier.

In certain aspects, the disclosed pharmaceutical compositions comprise the disclosed peptides (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The peptides described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed peptide (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### G. Methods of Treating

Disclosed are methods of treating a subject having a cancer with high TM4SF1 expression comprising administering a therapeutically effective amount of a composition comprising a T cell genetically modified to express one or more of the CAR polypeptides disclosed herein to a subject in need thereof. In some aspects, cancers having high TM4SF1 expression can be, but are not limited to, bladder cancer, ovarian, cholangiocarcinomas, colorectal cancers. In some aspects, an increase in TM4SF1 expression is compared to urothelial cancer cells, or any other cancer known to not overexpress TM4SF1. In some aspects, an increase in TM4SF1 expression is compared to non-cancerous tissue from a subject with the same cancer.

Disclosed are methods of treating bladder cancer comprising administering a therapeutically effective amount of a composition comprising a T cell genetically modified to express one or more of the CAR polypeptides disclosed herein to a subject in need thereof. In some aspects, the T cell comprises a CAR polypeptide that binds human TM4SF1, wherein the T cell has increased specificity to bladder cancer cells.

Also disclosed are methods of treating bladder cancer comprising administering an effective amount of a T cell genetically modified to express a CAR polypeptide comprising a TM4SF1 antigen binding domain, a hinge and transmembrane domain, and an intracellular signaling domain.

In some aspects, the bladder cancer is a histologic variant subtype of bladder cancer.

In some aspects, the subject in need thereof has higher levels of expression of TM4SF1 compared to subjects with urothelial cancer

Disclosed are methods of treating a subject having a cancer with high TM4SF1 expression comprising administering an effective amount of at least one of the disclosed vectors to the subject in need thereof. For example, disclosed are methods of treating bladder cancer comprising administering an effective amount of a vector comprising the nucleic acid sequence capable of encoding a disclosed CAR polypeptide to a subject in need thereof. In some instances, the vectors can comprise targeting moieties. In some instances, the targeting moieties target T cells.

Disclosed are methods of treating a subject having a cancer with high TM4SF1 expression comprising administering an effective amount of a composition comprising one or more of the disclosed antibodies or fragments thereof. For example, disclosed are methods of treating bladder cancer comprising administering an effective amount of a composition comprising an antibody or fragment thereof comprising SEQ ID NO:87. Disclosed are methods of treating bladder cancer comprising administering an effective amount of a composition comprising an antibody or fragment thereof comprising a variable heavy chain comprising a sequence having at least 90% identity to a sequence set forth in SEQ ID NOs: 88, 63, 64, 65, 66, 67, 68, 69, 70, 71, 72, or 73; a variable light chain comprising a sequence having at least 90% identity to a sequence set forth in SEQ ID NOs: 74, 75, 76, 77, 78, 79, 80, 81, 82, 83, 84, or 85; or both.

Disclosed are methods of treating bladder cancer comprising administering an effective amount of a composition comprising a CAR polypeptide comprising a TM4SF1 antigen binding domain, a transmembrane domain, and an intracellular signaling domain, wherein the TM4SF1 binding domain comprises a heavy chain variable domain comprising a CDR3 domain comprising an amino acid sequence that has at least 75% identity to SEQ ID NO: 1, 2, 3, 4, 5, 6, 7, 8, 9, 10, 11, or 12; a CDR2 domain comprising an amino acid sequence that has at least 75% identity to SEQ ID NO: 13, 14, 15, 16, 17, 18, 19, 20, 21, 22; and a CDR1 domain comprising an amino acid sequence that has at least 75% identity to SEQ ID NO: 23, 24, 25, 26, 27, 28, 29, 30, 31; and a light chain variable domain comprising a CDR3 domain comprising an amino acid sequence that has at least 75% identity to SEQ ID NO: 32, 33, 34, 35, 36, 37, 38, 39, 40; a CDR2 domain comprising an amino acid sequence that has at least 75% identity to SEQ ID NO: 41, 42, 43, 44, 45, 46, 47, 48, 49; and a CDR1 comprising an amino acid sequence that has at least 75% identity to SEQ ID NO: 50, 51, 52, 53, 54, 55, 56, 57, 58, 59, 60, 61, 62.

In some aspects, the disclosed methods of treating can comprise administering any one or more of the disclosed vectors, nucleic acid sequences, compositions or CAR polypeptides.

In some instances, the disclosed methods of treating bladder cancer further comprise administering a therapeutic agent. In some instances, the therapeutic agent can be, but is not limited to, chemotherapy (e.g., gemcitabine, methotrexate, vinblastine, doxorubicin, cisplatin, and mitomycin C), radiation, immunotherapy (e.g., avelumab, nivolumab, pembrolizumab, dostarlimab (Jemperli), and bacille Calmette-Guerin (BCG)), targeted therapy (e.g., atezolizumab (Tecentriq), avelumab (Bavencio), enfortumab vedotin-ejfv (Padcev), erdafitinib (Balversa), nivolumab (Opdivo), pembrolizumab (Keytruda), and sacituzumab govitecan-hziy (Trodelvy)), gene therapy (e.g., Nadofaragene firadenovec (Adstiladrin)).

### H. Methods of Killing

Disclosed are methods of killing TM4SF1 positive cells comprising administering an effective amount of a T cell genetically modified to one or more of the CAR polypeptides disclosed herein to a sample comprising TM4SF1 positive cells. In some aspects, TM4SF1 positive cells can be cancer cells having high TM4SF1 expression which can be, but are not limited to, cells from bladder cancer, ovarian, cholangiocarcinomas, colorectal cancers. In some aspects, the T cell comprises a CAR polypeptide that binds human TM4SF1, wherein the T cell has increased specificity to bladder cancer cells.

Disclosed are methods of killing TM4SF1 positive cells comprising administering an effective amount of a T cell genetically modified to express a CAR polypeptide comprising a TM4SF1 antigen binding domain, a hinge and transmembrane domain, and an intracellular signaling domain. In some aspects, the T cell comprises a CAR polypeptide that binds human TM4SF1, wherein the T cell has increased specificity to bladder cancer cells.

In some aspects, the sample is derived from a subject having bladder cancer. In some aspects, the sample is derived from a subject having any cancer having high TM4SF1 expression. In some aspects, cancers having high TM4SF1 expression can be, but are not limited to, ovarian, cholangiocarcinomas, colorectal cancers.

In some aspects, the disclosed methods of killing can comprise administering any one or more of the disclosed vectors, nucleic acid sequences, compositions or CAR polypeptides.

### I. Methods of Activating T cells

Disclosed are methods of activating a T cell expressing one of the CAR polypeptides disclosed herein comprising culturing the T cell with a cell expressing TM4SF1 and detecting the presence or absence of IFN-γ after culturing, wherein the presence of IFN-γ indicates the activation of the T cell. In some aspects, the T cell comprises a CAR polypeptide that binds human TM4SF1, wherein the T cell has increased specificity to bladder cancer cells

### J. Methods of Making Cells

Disclosed are methods of making a cell comprising transducing a cell with one or more of the disclosed vectors. In some instances, the cell can be, but is not limited to, T cells or NK cells. In some instances, the T cell can be a γδ T cell or an αβ T cell. In some aspects, the cell is a αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Disclosed are methods of making a cell comprising transducing a T cell with one or more of the disclosed vectors. For example, disclosed are methods of making a cell comprising transducing a T cell with a vector comprising the nucleic acid sequence capable of encoding a disclosed CAR polypeptide to a subject in need thereof.

### K. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the CAR polypeptides or nucleic acid sequences disclosed herein. In some aspects, disclosed are kits comprising one or more of the cells disclosed herein.

Also disclosed are kits comprising one or more of the CAR polypeptides, nucleic acids, vectors or cells disclosed herein.

### EXAMPLES

### a. Single Cell Analysis of Histologic Variants in Bladder Cancer Reveals an Aggressive CA125+ Tumor Cell State and TM4SF1 as Targetable Molecular Feature

### 1. Methods

i. Sample Collection.

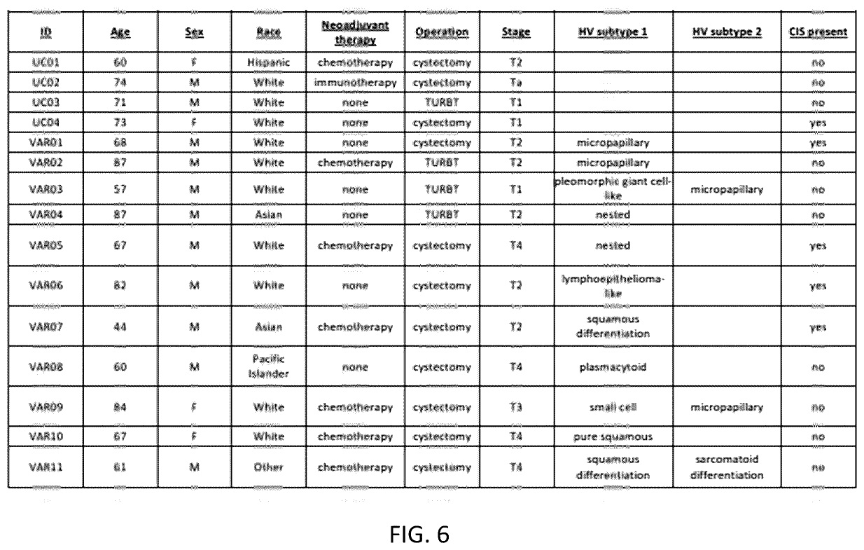

We obtained a total of 15 fresh bladder tumor samples from patients undergoing surgery at our institution under IRB 10-04057. In patients undergoing transurethral resection of bladder tumor (TURBT), specimens were obtained using cold biopsy forceps. In patients undergoing radical cystectomy, specimens were obtained immediately upon removal of the bladder to minimize the effects of ischemia. Visible tumor was excised from the specimen after the bladder was opened according to standard pathology protocol. Clinical and pathological data are shown in FIG. 6. All tissue was immediately placed in RPMI 1640 media on ice.

ii. Tissue Dissociation.

Mechanical tissue dissociation was performed using scissors and enzymatic dissociation was performed using 1000 U/mL Type IV collagenase (Worthington, Cat: LS004188) at 37° C. for 30 minutes. A single cell suspension was isolated using a 40 μm strainer, pelleted at 300 g, and reconstituted in RPMI 1640 media with 10% FBS. Viability and concentration were determined using acridine orange/propidium iodide on a LUNA automated cell counter (Logos Biosystems). The suspension was then adjusted for a target loading concentration of ˜50,000-100,000 live cells/mL.

iii. Single-Cell RNA Sequencing.

cDNA library preparation was performed using the Seq-Well platform as previously described.15,16 Briefly, 10,000-20,000 cells were loaded onto a Seq-Well array containing 110,000 barcoded mRNA capture beads (ChemGenes, Ct: MACOSKO-2011-10(V+)). Arrays were sealed using a polycarbonate membrane (Sterlitech, Cat: PCT00162X22100) at 37° C. for 40 minutes. Cells were then lysed in lysis buffer (5 M guanidine thiocyanate, 1 mM EDTA, 0.5% sarkosyl, 1% BME) for 20 minutes at room temperature. Hybridization of mRNA to the beads was performed in hybridization buffer (2 M NaCL, 4% PEG8000) for 40 minutes. The beads were then collected and washed with 2 M NaCl, 3 mM MgCl2, 20 mM Tris-HCl pH 8.0, 4% PEG8000.

Reverse transcription was then performed using Maxima H Minus Reverse Transcriptase (ThermoFisher, Cat: EP0753) in Maxima RT buffer, PEG8000, template switch Oligo dNTPs (NEB, Cat: No447L), and RNase inhibitor (Life Technologies, Cat: AM2696) at room temperature for 15 minutes and then 52° C. overnight. Second strand synthesis was performed using Klenow Exo-(NEB, Cat: M0212L) in Maxima RT buffer, PEG8000, dNTPs, and dN-SMRT oligo for 1 hr at 37° C. Whole transcriptome amplification was performed with KAPA HiFi Hotstart Readymix PCR kit (Kapa Biosystems, Cat: KK2602) and SMART PCR Primer (Supplementary Data). The reactions were purified using SPRI beads (Beckman Coulter) at 0.6× and then 0.8× volumetric ratio.

Libraries were prepared using 800-1000 pg of DNA and the Nextera DNA Library Preparation Kit. Dual-indexing was performed using N700 and N500 oligonucleotides. Library products were purified using SPRI beads at 0.6× and then 1× volumetric ratio. A final 3 nM dilution was prepared for each library and sequenced on an Illumina NovaSeq S4 flow cell.

iv. Sequencing and Alignment

Sequencing results were returned as paired FASTQ reads. These paired FASTQ files were then aligned against the hg19 reference genome (GRCh37.p13) using the dropseq workflow. The alignment pipeline output for each pair of FASTQ files included an aligned and corrected bam files, a digital gene expression (DGE) matrix text which was used for downstream analysis, and text-file reports of basic sample qualities such as the number of beads used in the sequencing run, total number of reads, alignment logs.

v. Single-Cell Quality Control and Clustering Analysis

Cells were clustered and analyzed using Seurat (v4.3.0) in R (v.4.3.1). Cells with fewer than 300 genes, 500 transcripts, or a mitochondrial gene content of 20% or greater were removed. Doublets were removed using DoubletFinder (v.2.0.3). UMI-collapsed read-count matrices for each cell were used for clustering analysis in Seurat. We followed a standard workflow by using the “LogNormalize” method that normalized the gene expression for each cell by the total expression, multiplying by a scale factor 10,000. To identify different cell types, we computed the standard deviation for each gene and returned the top 2,000 most variably expressed genes among the cells before applying a linear scaling by shifting the expression of each gene in the dataset so that the mean expression across cells was 0 and the variance was 1. Principal components analysis (PCA) was run using the previously determined most variably expressed genes for linear dimensional reduction and the first 100 principal components (PCs) were stored, which accounted for 47.04% of the total variance. For graph-based clustering, the top 75 PCs and a resolution of 0.5 were selected, yielding 36 cell clusters. Differentially expressed genes (DEGs) in each cluster were identified using the FindAllMarker function within the Seurat package and a corresponding p-value was given by the Wilcoxon's Rank Sum test followed by an FDR correction. In the downstream analysis, tumor cells from each patient were further clustered in a similar manner. For the individual patient clustering analysis, the number of PCs was determined by the statistical permutation test and the straw plot, and clustering resolution was selected accordingly.

vi. Cell-Type Annotation and Copy Number Variation

To annotate each cell type from the previous clustering, we referred to canonical markers and signature gene sets developed from established studies for each cell type. We computed the signature scores of these established gene sets for each cell in our dataset using the AddModuleScore function in Seurat. Each cluster in our dataset was assigned with an annotation of its cell type by top signature scores within the cluster. To validate the identities of the tumor cell populations, we estimated copy number variants (CNV) via InferCNV (Version 1.4.0), using all the non-tumor populations as reference. During the inferCNV run, genes expressed in fewer than five cells were filtered from the data set and the cut off was fixed at 0.1. Hidden Markov model (HMM) based CNV prediction was generated and estimated CNV events were shown in a heatmap.

vii. Pseudotime Analysis

To further investigate the differential trajectories of tumor cells in each patient, a pseudotime analysis was conducted in Monocle. To analyze gene expression relative to the Cluster 13 cell state, Cluster 13 cells were selected as the starting point for the pseudotime trajectory. Pseudotime trajectories were computed accordingly and visualizations were made to illustrate specific gene expression levels along the pseudotime trajectory in each patient.

viii. Gene Ontology and Gene Set Enrichment Analysis

Within the tumor cells, we created a customized gene set signature for each variant tumor cell population of interest. Using the DEGs obtained from FindAllMarker function, genes with log 2 fold change >2 and statistical significance (FDR q<0.05) were included in the customized signature gene set.

To assess the in silico functional roles of Cluster 13 cells, the signature gene sets derived from the scRNA-seq data to run gene ontology (GO) analysis against known signature gene set collections such as Hallmark, C2CP, C2CGP, C5GO and C6 oncogene collections was used. The gene ratio and statistical significance levels from the overexpression test were calculated. Normalized gene expression data and variant tumor types as metadata were used in the GSEA analysis run on the GSEA software.

To examine the association between signature gene sets or marker expression derived from the dataset and known basal/luminal signatures or canonical marker expression in the TCGA-BLCA bulk RNA sequencing dataset for validation, ssGSEA (single set Gene Set Enrichment Analysis) was performed by projecting the TCGA sample expression data onto the transcriptomic space defined by marker expression and established signature gene sets. For each target marker expression of target signature gene set, association was quantified via IC (information coefficient) and statistical significance was computed.

ix. Survival Analysis.

Within the TCGA-BLCA bulk RNA-seq dataset, the Cluster 13 signature score was computed on the normalized gene expression data for each sample. Samples were then divided into high and low groups based on the 20% percentile cutoff of the Cluster13 signature score. The overall survival (OS) distribution of both groups was compared by means of log-rank tests using the survfit function from the survival package (v3.3-1). A the Kaplan-Meier (KM) survival curve was plotted using the survminer (v0.4.7) package.

x. Histology and Immunohistochemistry.

FFPE bladder cancer tissue (from the scRNAseq cohort and additional specimens) banked under IRB 10-04057 was sliced to 4 μm and mounted on positively charged Superfrost microscope slides. Hematoxylin and eosin (H&E) staining was performed using a standard method. CA125 immunohistochemistry (IHC) was performed using a clinically validated mouse monoclonal antibody (Signet, clone OC125) on an automated Ventana Benchmark Ultra IHC system using CC1 cell conditioning solution. TM4SF1 IHC was performed using a rabbit polyclonal antibody (Abcam, ab113504) at a 1:500 dilution after a 10-minute citrate antigen retrieval at 100° C. on a Leica Bond III platform. A tissue microarray including pancreas, vascular endothelium, adipose, and lymphoid tissue was used for positive and negative control. The signal in tumor cells was compared with that of endothelial cells on the same slide; tumor cells that stained equally or darker than endothelial cells were scored as “strong” while those that stained lighter were scored as “weak.” All TM4SF1 and CA125 stains were reviewed by a pathologist.

xi. CA125 Serology

Serum CA125 levels were prospectively measured in patients undergoing TURBT or cystectomy for bladder tumors using the Abbott Architect Chemiluminescent Microparticle Immunoassay (CMIA) and reviewed under IRB 10-04057. Blood samples were drawn in the preoperative area prior to surgery. Pathologic diagnoses were reviewed. Tumors with >5% HV components were categorized as “HVs” while tumors with no mention of HV were categorized as “UC.” Tumors with equivocal or negligible HV components were excluded from the analysis; patients with “no tumor” on final pathology were also excluded.

xii. CAR Constructs

The heavy (VH) and light (VL) chains of the TM4SF1 scFv binder was obtained from antibody AGX-A01 (patent U.S. Ser. No. 01/208,495B2). The VH and VL sequences were cloned in two configurations using the Gibson Assembly protocol (Twist) into a CAR backbone containing IgG4 spacers, CD8 hinge and transmembrane domain, 4-1BB costimulatory domain, CD3ζ chain, and EGFP. Plasmids were prepared using the NucleoBond Xtra Midi Plus kit (Takara Bio).

xiii. CAR Lentivirus Production

For TM4SF1-CAR lentivirus production, HEK293T-Lenti-X cells (Takara Bio) were thawed, cultured, and expanded in DMEM media supplemented with 10% FBS. HEK293T-Lenti-X cells were transfected with the TM4SF1-CAR lentiviral plasmid and the packaging plasmids psPAX2 and pVSVg using the TransIT-LT1 transfection reagent (Mirus Bio). Cell supernatant was collected at 48 hours and 72 h. The virus was filtered and concentrated using the Lenti-X Concentrator (Takara Bio) according to manufacturer's instructions and resuspended in serum-free media.

xiv. TM4SF1-CAR T Generation

Human T cells were isolated from a leukopak (Stemcell Technologies) using the Easy Sep Human T cell enrichment kit (Stemcell technologies). T cells were then plated on retronectin coated plates (Takara, T100A), stimulated with Human CD3/CD28 T Cell Activator (Stemcell Technologies, 10971) per million cells, and concentrated lentivirus was added. Cells with virus were spun at 1000 rpm for 45 minutes. After 72 hours of incubation, virus was removed, and cells were allowed to recover for 2-3 days. Transduction efficiency was evaluated via flow cytometry by GFP expression. If less than 30% of the T cells were GFP positive, the cells were MACs sorted using a biotinylated c-myc antibody (Miltenyi Biotec, 130-124-877) and isolated using the MiniMACS separator and columns (Miltenyi) according to manufacturer's protocol. The CAR-T cells were grown in either ImmunoCult-XF T Cell Expansion Medium (Stemell Technologies, 01981) or TexMACS™ Medium (Miltenyi Biotech, 130-097-196). Human recombinant IL-15 (Stemcell Technologies, 78031) and IL-7 (Stemcell Technologies, 78053), 10 ng/mL final concentration each was freshly added to the cells every 2-3 days, with cells grown at a concentration of 1×10⁶ per mL and used between day 14-20.

xv. Cell Culture

5637 cells were obtained from the UCSF Cell Culture Facility. UMUC-3 cells were a gift from Bradley Stohr (UCSF). T24, UMUC-1 and 253JBV cells were gifts from Peter Black (University of British Columbia) and David McConkey (Pathology Core, Bladder Cancer SPORE, MD Anderson Cancer Center). Cells were grown in standard MEM media (Corning) supplemented with 10% FBS (Seradigm) and penicillin/streptomycin. All experiments were conducted within 20 passages from the parental stock. Cells were validated by STR profiling and routinely tested for mycoplasma (Lonza).

xvi. TM4SF1 Knockout Cells

UMUC-3 TM4SF1-KO cells were generated by transient transfection (Lipofectamine 3000) of UMUC-3 cells with PX458 (Addgene, #48138). Each plasmid contained one of three different sgRNA targeting sequences: 1) AGTGCACTCGGACCATGTGG (SEQ ID NO:89); 2) GGTGTAGTTCCACTGGCCGA (SEQ ID NO:90); 3) ATTAGCCGCGATGCACAGGA (SEQ ID NO:91). 48-72 h after transfection, GFP-positive cells were sorted by FACS (BD Fusion) and expanded. Cells were then stained with a TM4SF1 antibody (Miltenyi, clone REA851, 1:100), sorted a second time by FACS (BD Fusion), and negative cells were collected and expanded.

xvii. TM4SF1 Flow Cytometry

Flow cytometric quantification of TM4SF1 expression across human bladder cancer cell lines was performed by incubating with anti-TM4SF1-PE antibody (Miltenyi, clone REA851, 1:100) for 30-60 minutes on ice. Cells were analyzed using an Attune NxT Flow and the median fluorescence intensity (MFI) was calculated and data were analyzed using FlowJo software.

xviii. IncuCyte Co-Culture Assays

Bladder cancer cells labeled with NucLightRed (Sartorius) were co-cultured with human non-transduced (NTD) T cells or TM4SF1-CAR T cells at variable effector-to-target (E:T) ratios. On day 0, 2000-5000 target cells were plated and allowed to adhere overnight. On day 1, effector T cells were added and tumor cell killing was monitored on an IncuCyte S3 (Sartorius). Images were obtained every 3-6 hours over 72-96 hours, and target cells were quantified based on the red object count or red area confluence and normalized to the starting day 1 values, and plotted on Prism (GraphPad, v10).

xix. Animal Studies

NSG (NOD/SCID/gamma) mice were housed in the UCSF barrier facility under pathogen-free conditions and were obtained through an in-house breeding core. For subcutaneous xenografts, 1×10⁶ cells were injected into the left flank of 8-10 week old male NSG mice. The injected cells were resuspended in 1:1 serum-free media and Matrigel (BD Biosciences). Mice were enrolled into treatment groups once tumor volumes reached between 50-100 mm3, typically 10-14 days after tumor cell inoculation. An intravenous injection of 3-5×10⁶ nontransduced (NTD) control or TM4SF1 CAR T cells was then delivered through the tail vein. Tumors were measured with digital calipers and mice were weighed twice weekly in a blinded fashion. Tumor volumes were recorded using Studylog Animal Study Workflow software and plotted using Prism (GraphPad, v10). Mice were euthanized when tumors reached 20 mm in any direction. For survival analysis, a log-rank test was used to compare the overall survival of mice in each cohort.

### 2. Results

i. Single Cell Analysis of Tumor Epithelial Cells Reveals a Novel CA125+ Tumor Cell State in Histologic Variants.

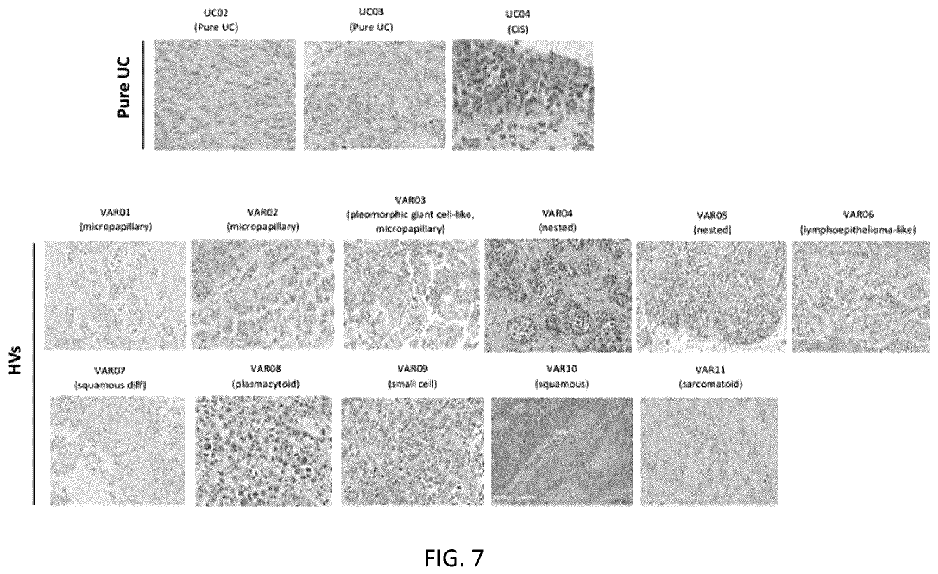

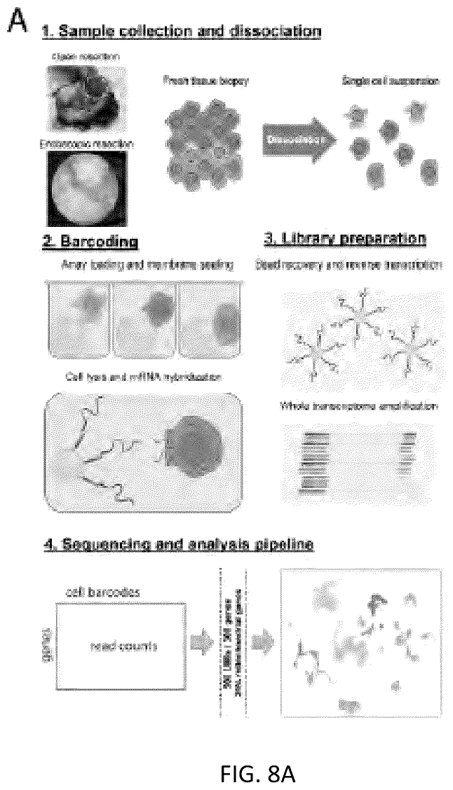

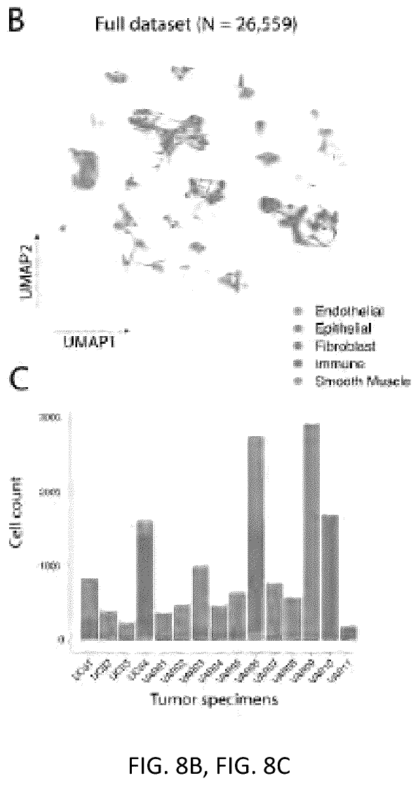

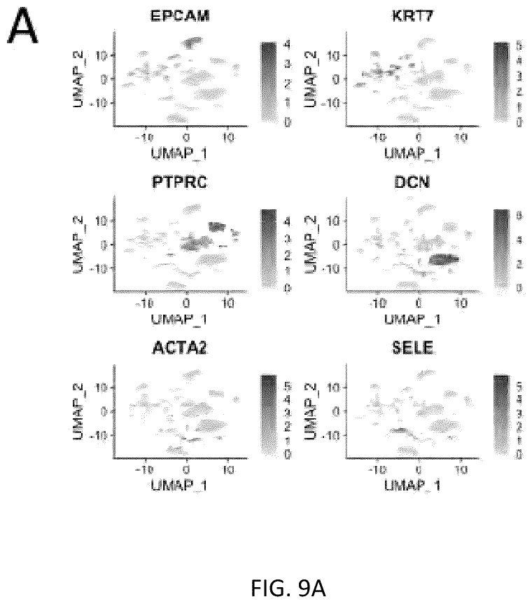

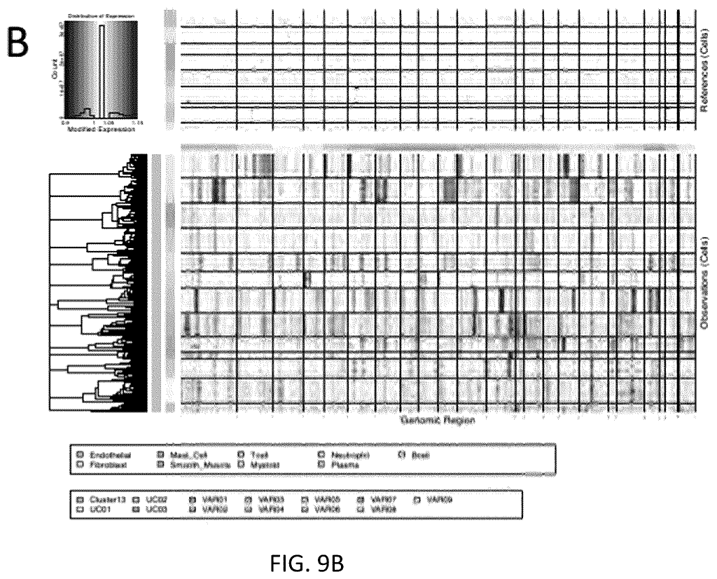

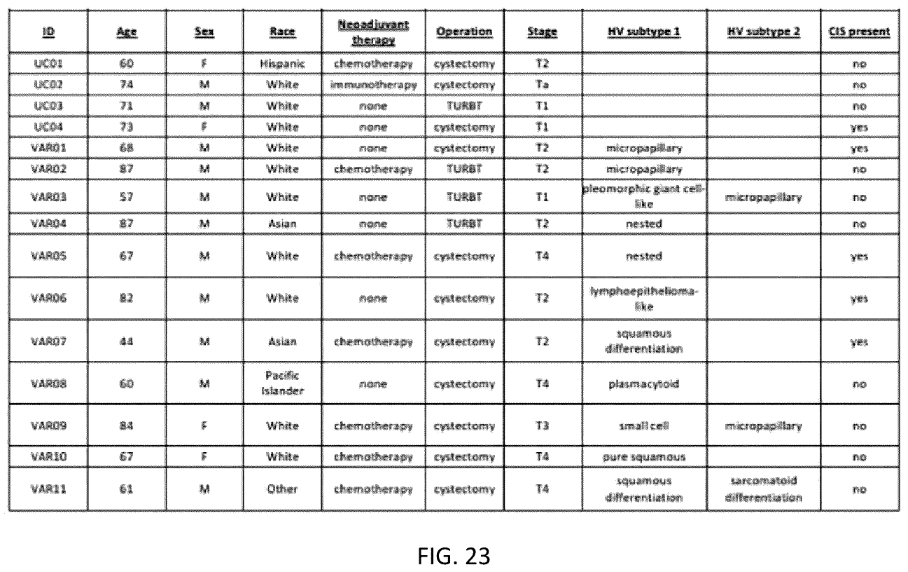

Tissue and dissociated single cells were collected from 4 pure urothelial carcinomas (UC) and 11 variant tumors. Detailed clinical information is displayed in FIG. 23; pathologic diagnoses were confirmed in specimens collected for sequencing (FIG. 7). Single-cell RNA sequencing (scRNA-seq) was performed using the Seq-well platform, and the sequencing results were processed in the customized analytical pipeline (FIG. 8A). After ambient RNA decontamination and removal of low-quality cells, 21,533 cells in total were captured for downstream analysis from these specimens (FIG. 8B). While tumor epithelial cells were captured from almost all tumors, the capture rate for stromal and immune cells was highly variable among the specimens (FIG. 8C) per the annotation based on graphical clustering patterns and canonical cell-type specific markers for tumor epithelial/urothelial cells (EPCAM, KRT7), immune cells (PTPRC), stromal cells (DCN, ACTA2), and endothelial cells (SELE) (FIG. 9A).

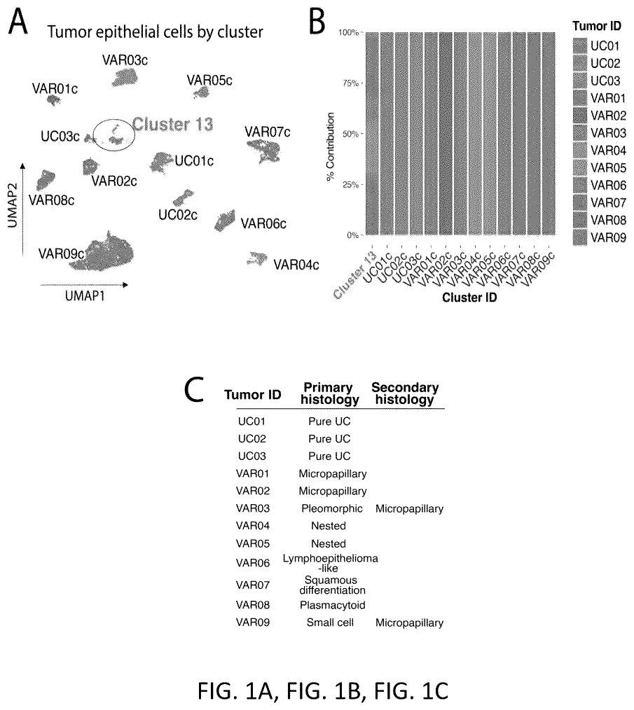

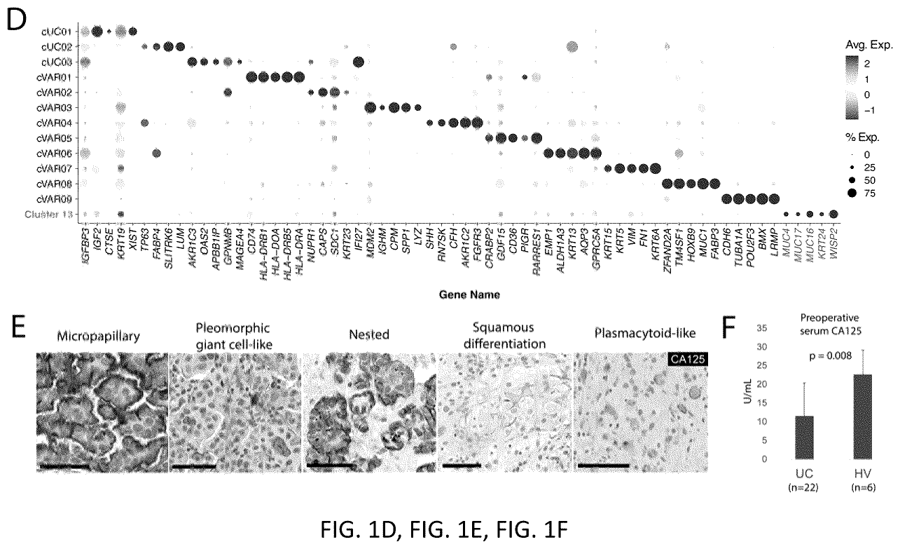

The analysis was focused on tumor cell biology by subsetting and re-clustering the tumor epithelial cells from the main dataset (FIG. 1A). Three tumors were excluded that did not meet a threshold of 150 tumor epithelial cells for analysis (UC04, VAR10, VAR11). Although neuroendocrine tumors are generally considered non-urothelial cancers, the tumor with small cell HV (VAR09) was included due to the presence of urothelial components within the tumor (carcinoma in situ and micropapillary variant). The final tumor epithelial dataset thus included three pure UCs (UC01-UC03) and nine HVs (VAR01-VAR09). To confirm the tumor content in this dataset, InferCNV was used to estimate the copy number profiles of all epithelial cell clusters using stromal and immune cells as reference (FIG. 9B).

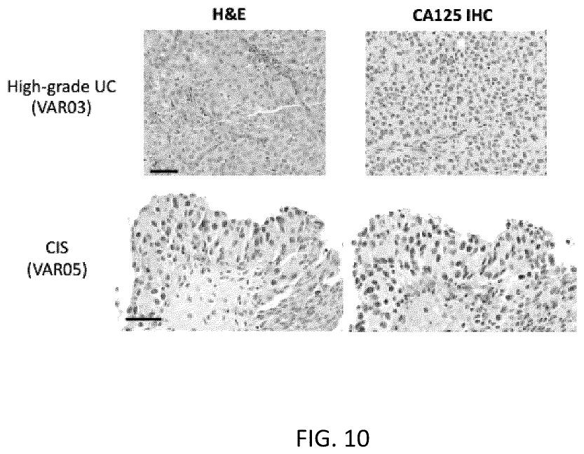

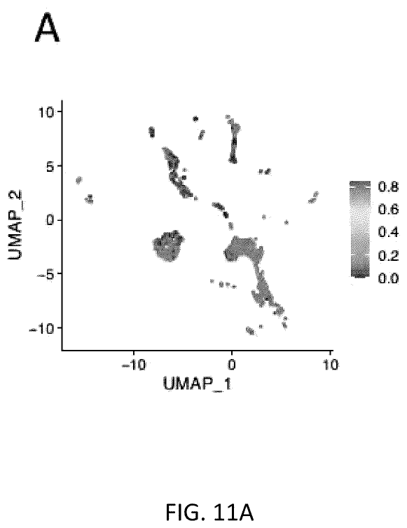

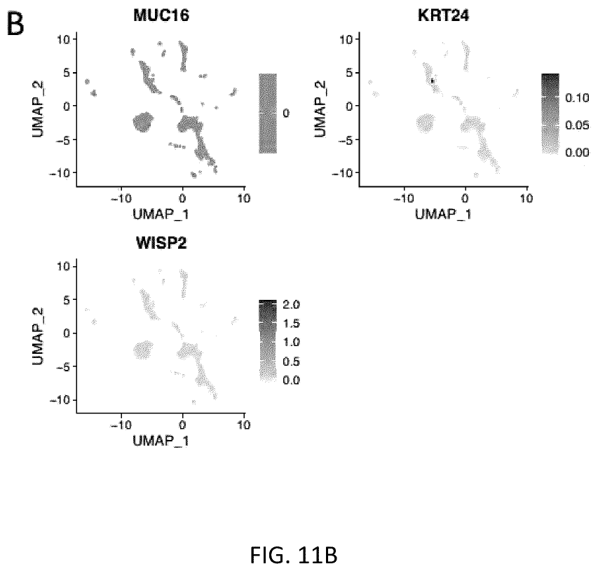

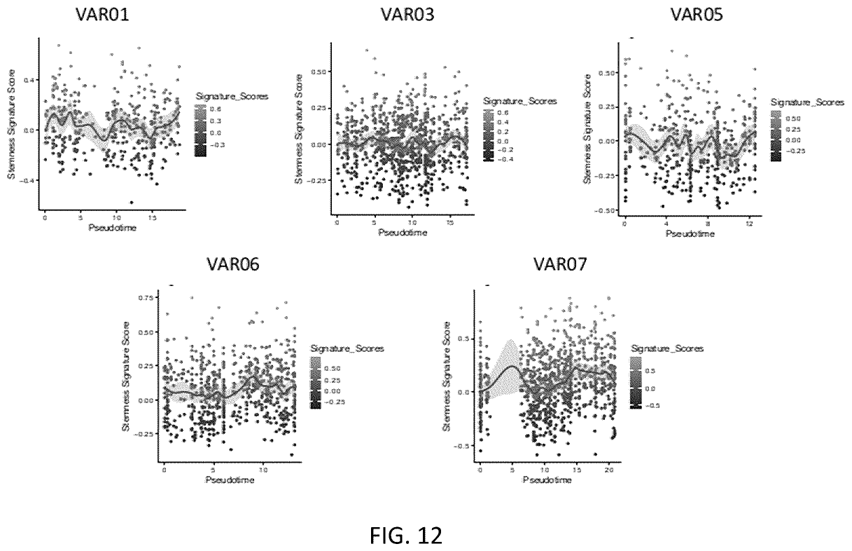

Most tumor cells formed their own clusters corresponding to the tumor of origin and were named accordingly, i.e. VAR01c is the predominant cluster obtained from the VAR01 tumor (FIG. 1A). Interestingly, one cluster, which we named “Cluster 13” based on the number assigned by the clustering algorithm, was comprised of cells from multiple HV tumors (FIG. 1B-C). Differentially expressed genes (DEGs) for each tumor cluster were computed and curated, and MUC16, WISP2, KRT24, MUC17, and MUC4 were among the top DEGs for Cluster 13 (FIG. 1D). To validate the existence of Cluster 13 cells histologically, we performed immunostaining of CA125 (encoded by MUC16) in HV (N=14) and UC (high-grade invasive and carcinoma in situ) tumors (N=20). A subpopulation of CA125+ cells was found in a variety of HV tumors with different subtypes (13/14) (FIG. 1E) but rarely in tumors with UC (1/11) or carcinoma in situ (CIS) histology (1/9). In tumors with mixed HV and UC components such as VAR03 and VAR05, CA125+ cells were present in the HV regions (FIG. 1E, pleomorphic giant cell-like, nested) but absent in the high-grade UC and CIS regions (FIG. 10). The Cluster 13 signature or expression of MUC16, KRT24, and WISP2 was not detected in a previously published bladder cancer scRNA-seq dataset derived from UC bladder tumors (FIG. 11A-B). The results indicate that the cancer cells found in Cluster 13 represents a tumor cell state highly specific to -, but not exclusive to -, HV-containing tumors. To explore whether CA125 expression in these cells could be useful as a clinical biomarker, preoperative serum CA125 levels were assayed in bladder cancer patients undergoing surgery and found CA125 levels to be higher in those with HV components in their final pathology compared to those with UC only (22.7±6.6 U/mL vs 11.6±8.8 U/mL, p=0.008) (FIG. 1F).

ii. Cluster 13 Cells Exhibit Hallmarks of Transcriptional Convergence.

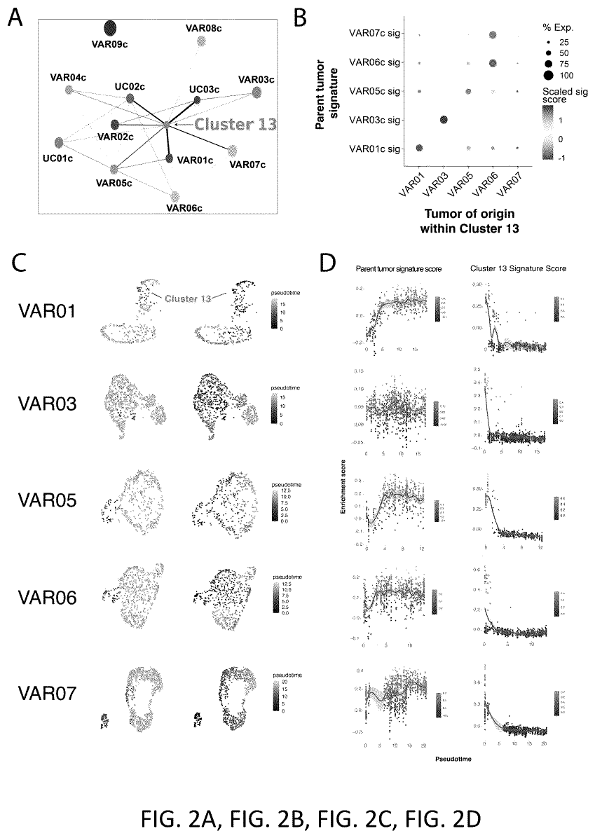

To investigate the overall transcriptomic relationship among tumor clusters and to test whether similar HV subtypes share gene expression programs (e.g. micropapillary to micropapillary, nested to nested), an unsupervised partition-based graphical abstraction (PAGA) graph was generated. While no prominent subtype-specific associations were seen, Cluster 13 cells formed a central node with an association to almost every other tumor cluster, even to those whose parent tumor did not contribute any cells to Cluster 13 (FIG. 2A). This result raised the possibility that Cluster 13 represents either a convergent cell state or a common progenitor cell state.

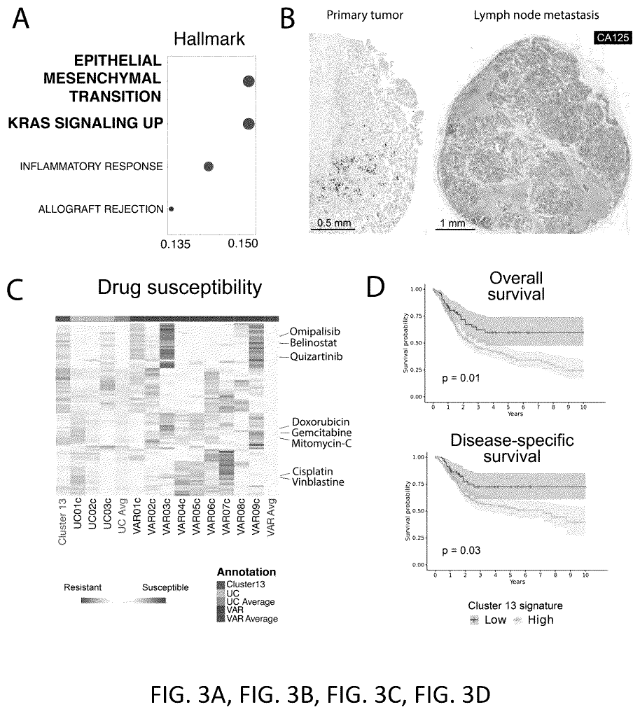

The relationship between the Cluster 13 cells and the parent tumor cells was investigated. Cluster 13 cells bear the signature of the parent tumor with a high degree of specificity, indicating the likelihood that all cells within these tumors are clonally related (FIG. 2B). Pseudotime analysis was performed for VAR01, VAR03, VAR05, VAR06, and VAR07 (FIG. 2C). The Cluster 13 cells were arbitrarily selected as the starting point for the pseudotime trajectory in each tumor to evaluate the relative signature enrichment between Cluster 13 cells and parent tumor cells (FIG. 3C). The Cluster 13 signature was anticorrelated with the parent tumor signature in four of five tumors (FIG. 3D), and the marked contrast of the Cluster 13 signature along the pseudotime in all five tumors indicates that Cluster 13 arises as a derivative of the parent tumor rather than vice versa. To further test the possibility that Cluster 13 is a progenitor cell state rather than a derivative tumor cell state, a nine-gene bladder stem cell signature (PROM1 (CD133), POU5F1 (Oct4), SOX2, ALDH1A1, SOX4, EZH2, YAP1, CD44, and KRT14) was generated based on previous studies in bladder cancer stem cells; and found no significant enrichment of this signature in Cluster 13 cells (FIG. 12). While scRNAseq alone cannot prove the temporal relationship between these cells, the results support the idea that cancer cells found in Cluster 13 are a convergent cell state in HV tumors.

iii. Cluster 13 Cells Harbor Adverse Molecular Features.

Gene ontology (GO) analysis was performed on the DEGs for Cluster 13 cells and revealed a significant enrichment in epithelial-to-mesenchymal transition (EMT) and KRAS signaling gene sets (FIG. 3A). These findings raised the possibility that the Cluster 13 cells have more aggressive metastatic potential compared to non-Cluster 13 cells. Using CA125 again as a putative marker for Cluster 13 cells, CA125 staining was examined in five HV tumors with lymph node metastases and observed a striking homogeneous enrichment of CA125+ cells in the lymph nodes compared to the primary tumor in 4 of 5 cases (FIG. 3B).

The susceptibility of Cluster 13 cells to chemotherapy and targeted agents in silico was examined. By training a drug response model using the Cancer Drug Response prediction using a Recommender System (CaDRReS) based on the Cancer Cell Line Encyclopedia (CCLE) database and Genomics of Drug Sensitivity in Cancer (GDSC) database, the estimated efficiency (percentage of tumor cells killed) for drugs from the GDSC database was inferred for each tumor cluster in the scRNA-seq dataset. The analyses revealed that Cluster 13 cells were more predicted to be more resistant to most chemotherapeutic agents, particularly in the case of conventional bladder cancer agents such as cisplatin, gemcitabine, and mitomycin C, compared to UC and non-Cluster 13 HV cells (FIG. 3C). Consistent with these adverse features, tumors that harbor higher Cluster 13 signature scores in TCGA-BLCA had worse overall survival and disease-specific survival (FIG. 3D).

Taken together, these results indicate that HV tumors contain a cancer cell state that is enriched in metastases and are predicted to be more resistant to chemotherapy. This cell state offers a potential mechanism to help explain why HV tumors are more aggressive than UC tumors.

iv. Histologic Variants Exhibit Transcriptional Hallmarks of Histologically Similar Non-Urothelial Cell Types

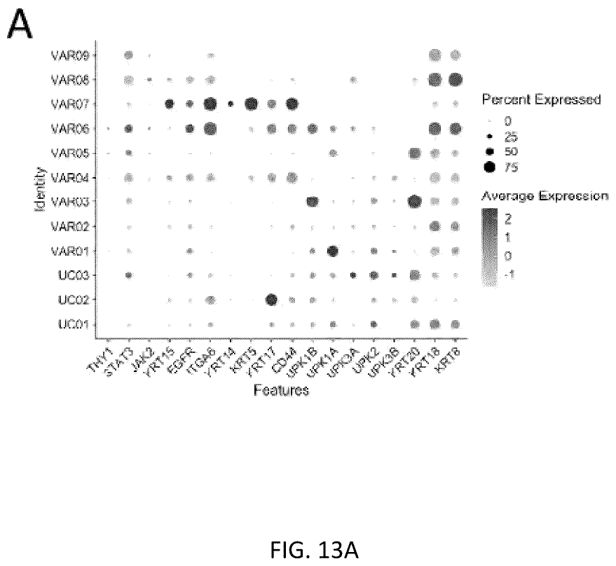

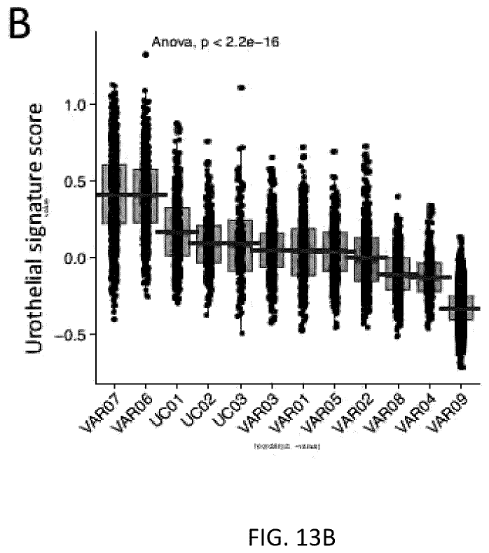

The analyses enabled us to examine whether HVs share molecular features with other histologically similar but non-urothelial cell types. Specifically, this possibility was investigated in tumor cells from VAR09 (small cell) and VAR08 (plasmacytoid), which exhibited low enrichment of urothelial differentiation genes (FIG. 13A-B).

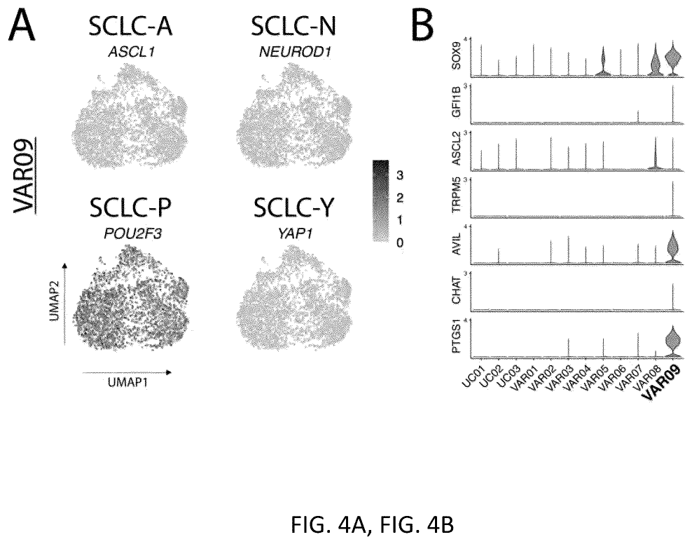

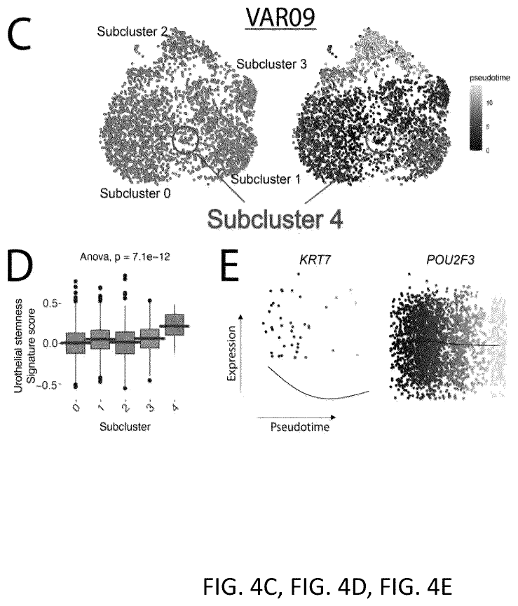

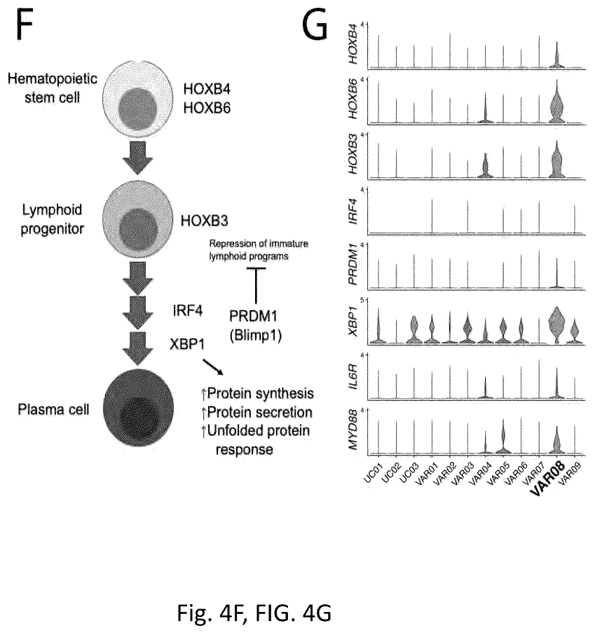

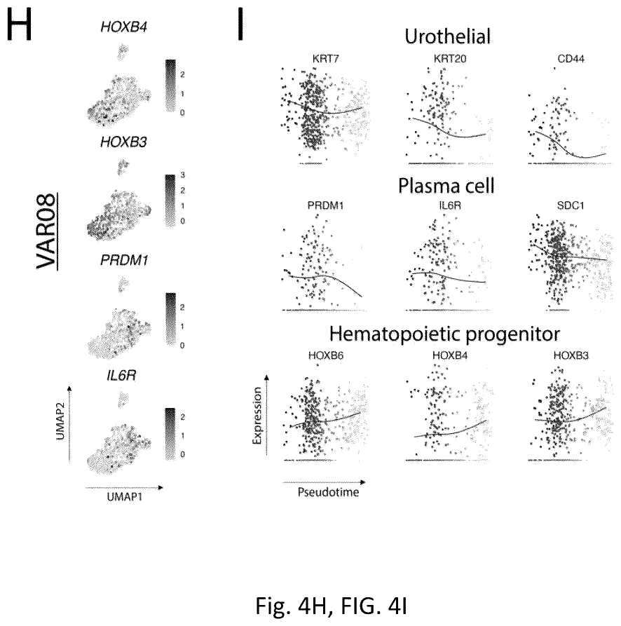

It has been proposed that small cell bladder cancers (SCBC) exhibit similarities with small cell lung cancers (SCLC) based on similar genomic alterations, but transcriptomic evidence has been limited. For VAR09, we applied a molecular subtyping schema based on the expression of ASCL1, NEUROD1, POU2F3, and YAP1 to the VAR09 tumor cells and noted enrichment in POU2F3, a gene associated with the SCLC-P subtype, or the “tuft cell-like variant” (FIG. 4A). POU2F3 expression was highly expressed throughout the VAR09 tumor cells along with downstream targets AVIL, SOX9, and PTGS1, expression of which was specific to VAR09 compared to other HVs. (FIG. 4B).

Although most VAR09 cells lacked expression of canonical urothelial markers, KRT7 expression was detected and was primarily localized to subcluster 4 (FIG. 4C). This cluster also harbored the highest stemness signature (FIG. 4D), so pseudotime analysis was performed using cells from subcluster 4 as a starting point, which showed a decrease in KRT7 expression along the pseudotime trajectory while POU2F3 largely remained constant (FIG. 4C, 4E). The coexistence of a KRT7+ and POU2F3+ cluster with progenitor-like features indicates the hypothesis that small cell bladder cancer cells arise from a urothelial origin.

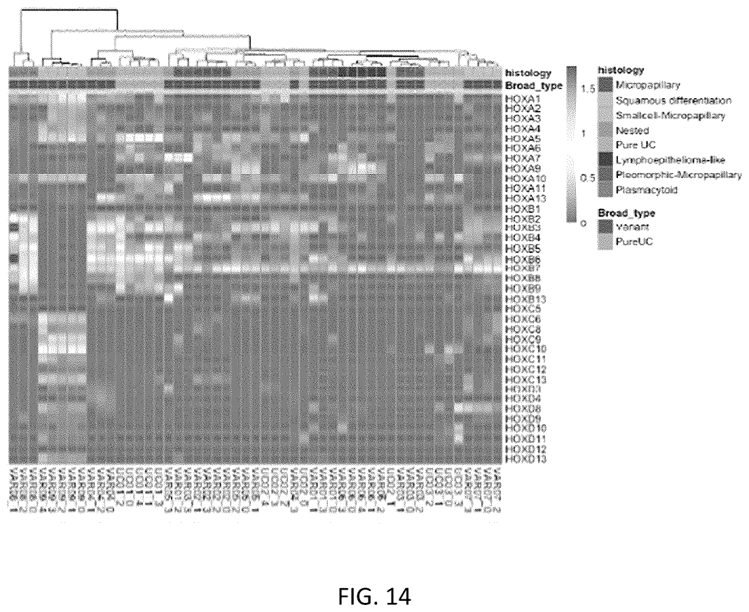

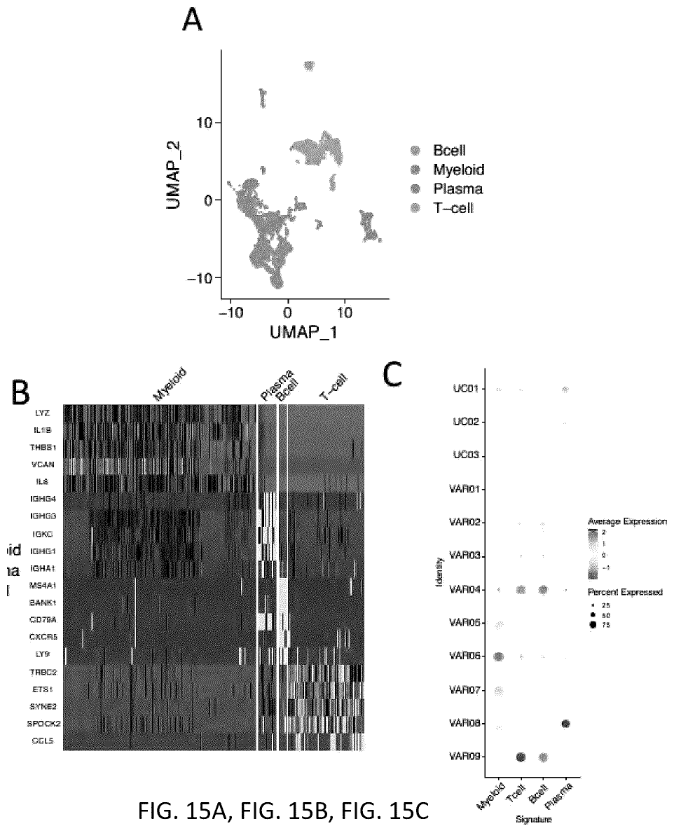

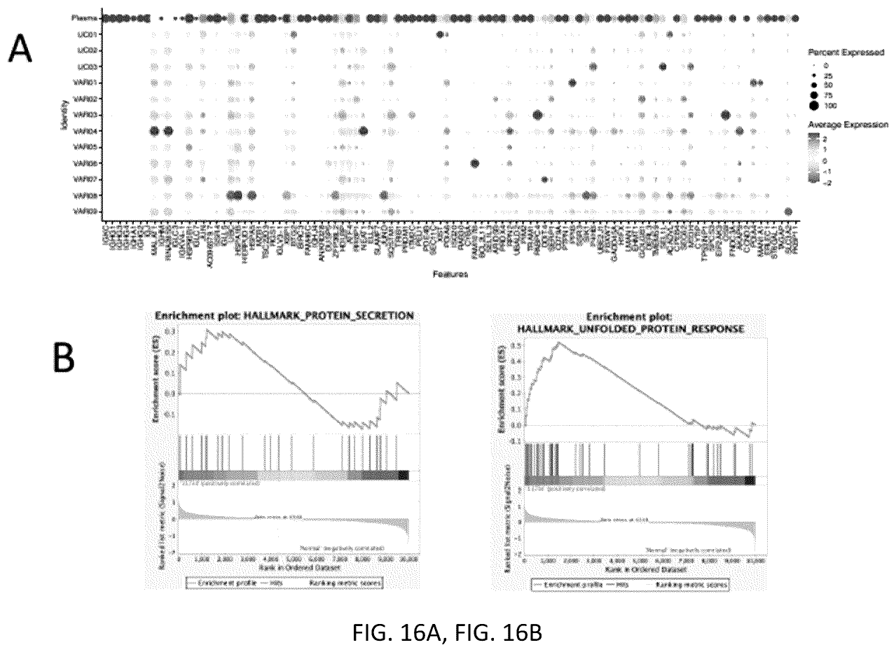

Due to their plasmacytoid appearance, whether tumor cells from VAR08 exhibited hallmarks of hematopoiesis and plasma cell differentiation due to their plasmacytoid appearance was investigated. HOX genes, transcription factors important for hematopoiesis that are known to be upregulated in some bladder cancers, were among the top DEGs for VAR08 (FIG. 2D). HOXB genes, which are required for hematopoietic stem cell (HSC) maintenance (HOXB4, HOXB6) and B-cell maturation (HOXB3), were enriched in VAR08 compared to other tumors (FIG. 14). Immune cell signatures derived from our scRNA-seq dataset were generated (Myeloid, T-cell, B-cell, and Plasma cell) (FIG. 15A-B), and it was found that VAR08 cells were enriched for the plasma cell signature (FIG. 15C). Expression of plasma cell-specific transcription factors PRDM1 and XBP1 and surface marker IL6R was elevated in VAR08 tumor cells, although the upstream activator IRF4 was notably absent (FIG. 16A). MYD88, a gene associated with lymphoplasmacytic lymphomas, was also detected in VAR08 tumor cells. The major determinants of plasma cell differentiation harbored within VAR08 were thus identified (FIG. 4F-G). Protein chaperones (HSPA1B, HSPA5) and protein synthesis genes (ELL2, EIF2AK3) were also highly expressed in tumor cells from VAR08 (FIG. 16A), and gene sets related to the unfolded protein response and protein secretion were also enriched (FIG. 16B). These features are consistent with the upregulation of downstream targets of PRDM1 and XBP1 similar to the transcriptional programs found in plasma cells.

Whether different stages of plasma cell maturation could be observed in this tumor was investigated. It was observed that HOXB4 and HOXB3, typically expressed earlier in the lineage, were anticorrelated with late genes IL6R and PRDM1, a broad repressor of immature transcriptional programs (FIG. 4H). This indicated the coexistence of a HSC-like state (HOXhigh) along with a more differentiated plasma-cell like state (PRDM1high/IL6Rhigh). When pseudotime analysis was performed starting from VAR08 cells with highest KRT7 expression, a surrogate for urothelial differentiation, a rise in HOX gene expression and a concomitant fall in the expression of KRT20, CD44, and SDC1 (CD138) (FIG. 4I) along the pseudotime trajectory was observed, indicating that VAR08 tumor cells may transition from a plasma cell-like urothelial state towards a more dedifferentiated HSC-like state. Of note, neither plasma cell lineage (CD34, PTPRC (CD45), CD19, MS4A1 (CD20), CD27, CD38) nor immunoglobulin gene expression was detected in VAR08, indicating that activation of hematopoietic transcriptional programs in urothelial cells does not necessarily result in expression of hematopoietic surface lineage markers.

v. TM4SF1 is a Surface Antigen Protein Broadly Enriched in Histologic Variant Tumor Cells

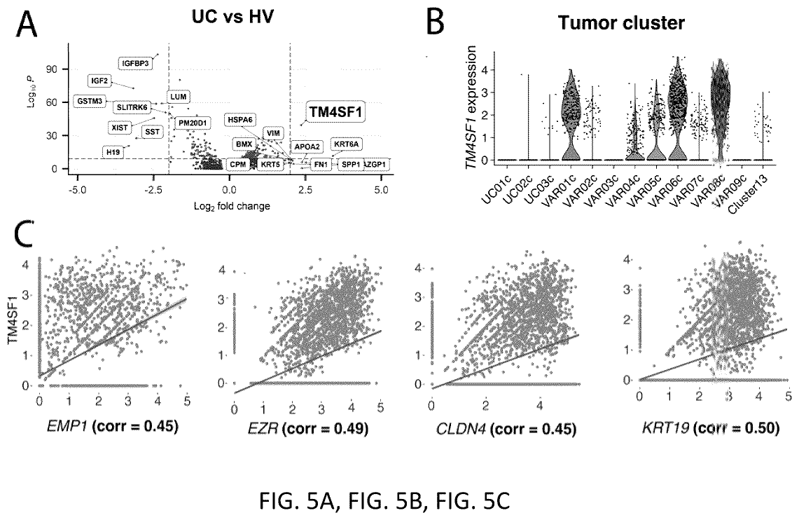

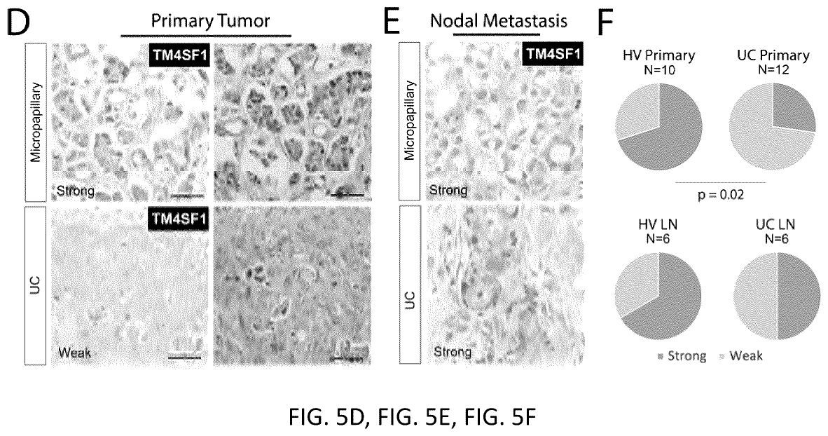

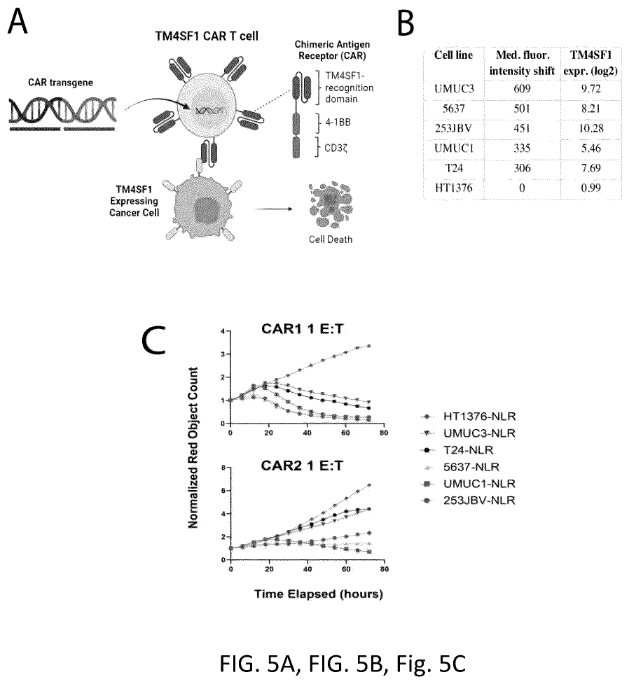

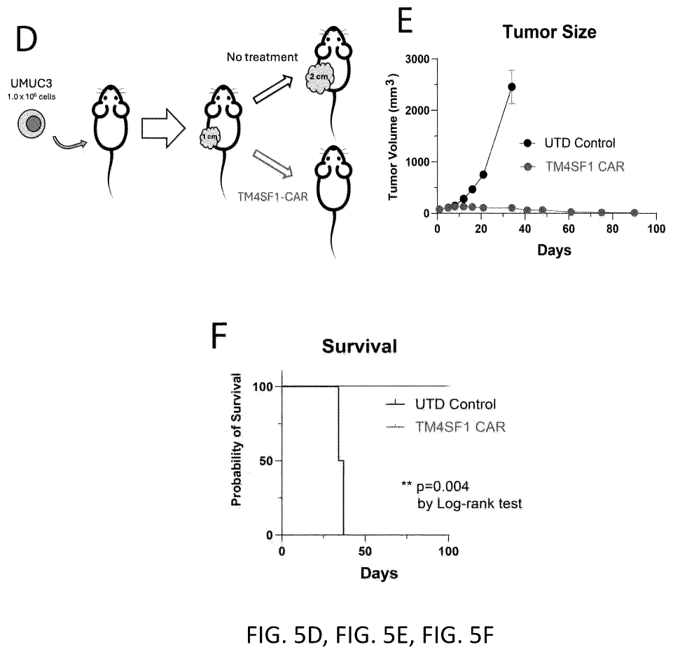

Having identified and characterized the Cluster 13 cell state in HVs, it was next asked whether our scRNA-seq results could help identify any molecular features broadly enriched in HV tumor cells compared to UCs; defining such features would facilitate the development of HV-specific targeted therapies. All tumor cells were categorized as HV or UC according to the histology of the parent tumor and computed the DEGs (FIG. 5A). TM4SF1, a gene implicated in bladder cancer as a cell cycle and apoptosis regulator, was the top DEG in the HV group. Most HV tumor clusters, including Cluster 13, exhibited higher expression of TM4SF1 compared to those from pure UC tumors (FIG. 5B).

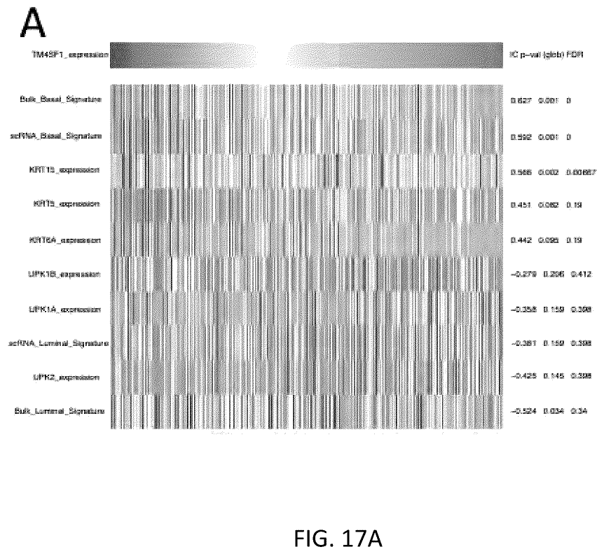

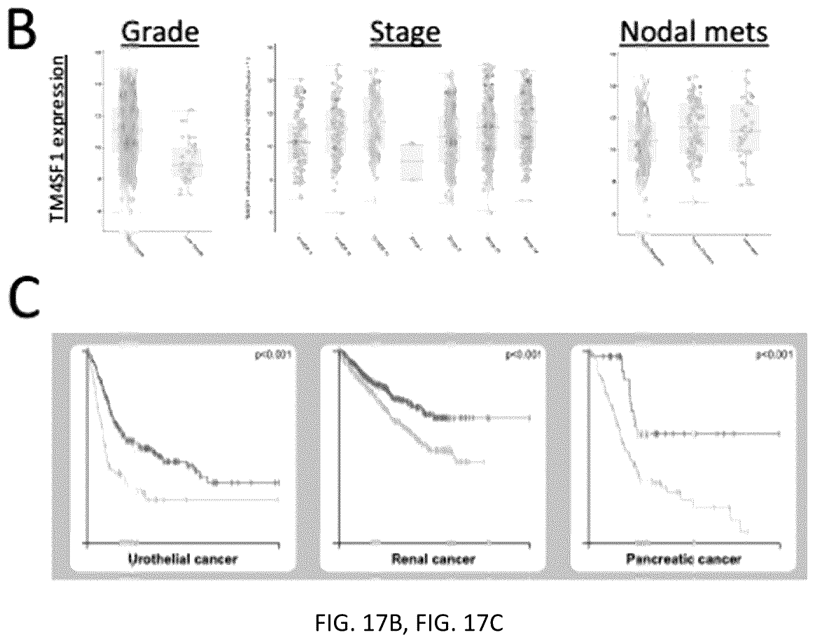

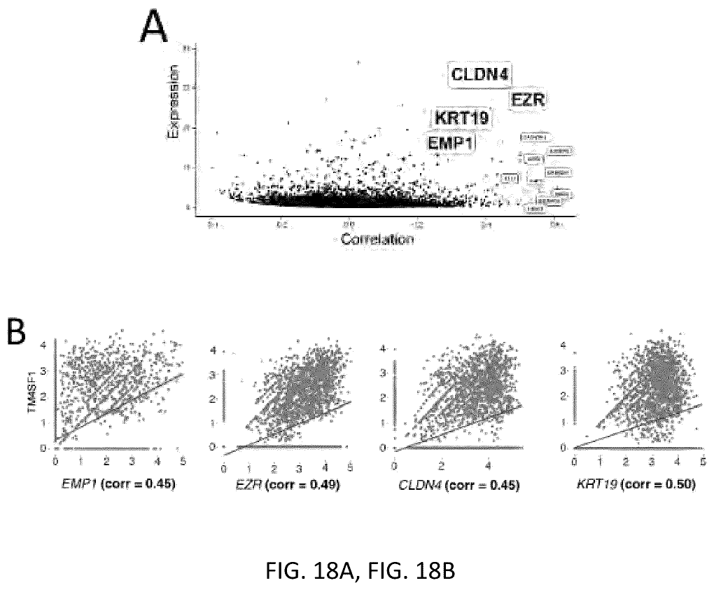

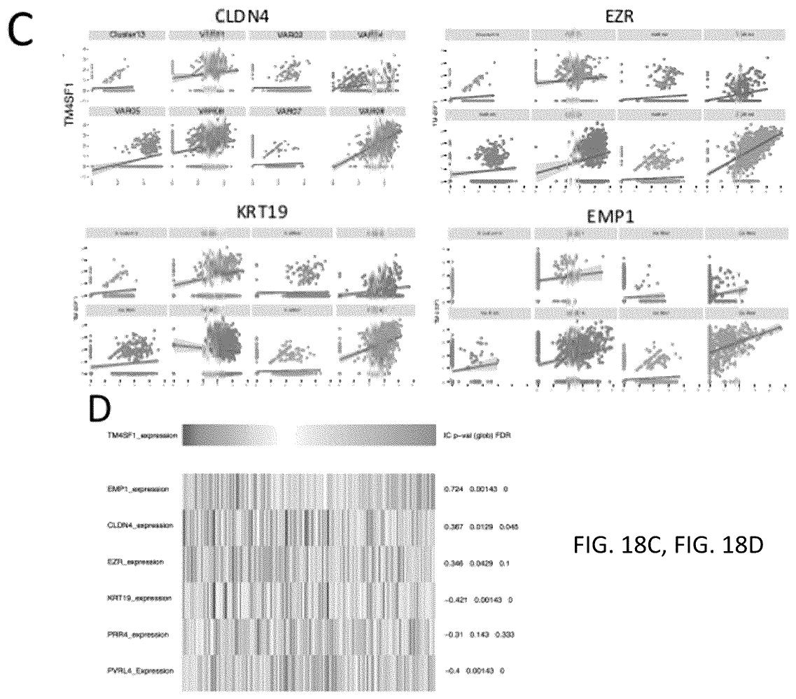

Consistent with previous reports, it was confirmed that high TM4SF1 expression is associated with basal tumor signatures (FIG. 17A) and adverse clinical outcomes in TCGA-BLCA (FIG. 17B-C).45 In our tumor epithelial data set, genes with the strongest positive correlation with TM4SF1 expression within the HV tumor cells were EMP1, CLDN4, EZR, and KRT19 (FIG. 18A-B). The associations within each TM4SF1-expressing tumor in our scRNA-seq dataset were checked and these were found to be positive and statistically significant in each case (FIG. 18C). EMP1, a gene implicated in cisplatin resistance and cancer recurrence, and CLDN4, a tight junction gene implicated in facilitating aggressive biology in bladder cancer, were also positively associated with TM4SF1 in TCGA-BLCA (FIG. 18D). Interestingly, a statistically significant association was not observed between TM4SF1 expression and SOX2, DDR1, MMP2, or MMP9 expression (data not shown), indicating that the expression of TM4SF1 in HVs may be regulated differently than what has been previously described in cell lines and nonurothelial cancers.

Using immunohistochemistry, TM4SF1 protein expression was validated in HV and UC cells, both in primary tumors and lymph node metastases (FIG. 5C-D). Consistent with the sequencing results, quantification of TM4SF1 staining using a binary “strong” and “weak” scoring system (see methods) demonstrated more frequent strong staining in HV primary tumors compared to UC primary tumors (p=0.02) (FIG. 5E).

vi. TM4SF1-CAR T Cells Demonstrate In Vitro and In Vivo Activity Against Bladder Cancer Cells.

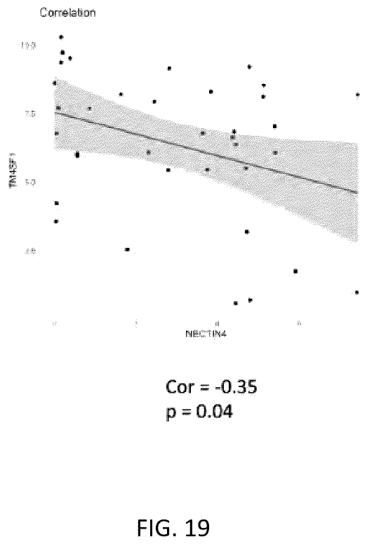

The enrichment of TM4SF1 expression in HVs and its cell surface expression made it a compelling candidate for developing a targeted therapeutic strategy. Expression of TM4SF1 is high across a number of tumor types, and its inverse correlation with PVRL4 (NECTIN4) expression in TCGA-BLCA and CCLE (FIG. 18D, FIG. 19) indicates that TM4SF1-directed therapies might be complementary to enfortumab vedotin (EV) therapy, an antibody-drug conjugate that targets NECTIN4 that was recently approved for frontline treatment of patients with locally-advanced/metastatic urothelial cancers.

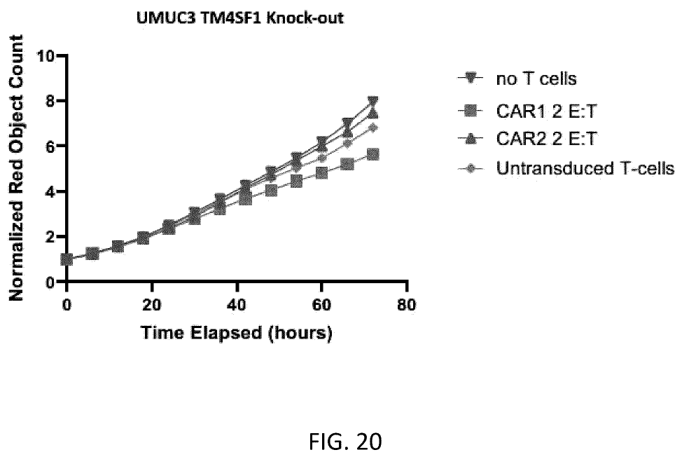

Given that there are no FDA-approved TM4SF1-directed therapeutic agents, it was next asked whether TM4SF1 could be targeted by chimeric antigen receptor (CAR) T cell therapy. To test this, a previously published TM4SF1 single-chain variable fragment (scFv) binder was used and incorporated this into a 41BB-based CAR bone in two configurations (VH-VL (CAR1) and VL-VH (CAR2)) (FIG. 6A). Both CAR T candidates were used against six bladder cancer cell lines with variable levels of endogenous TM4SF1 mRNA expression and surface protein expression (FIG. 6B). Whereas the TM4SF1-CAR T cells exhibited anti-tumor activity against bladder cancer lines expressing TM4SF1 (including UMUC3, T24, 5637, 253JBV and UMUC1), the TM4SF1-CAR T cells did not kill HT1376, which are negative for TM4SF1 (FIG. 6C). CAR1 had slightly better activity in vitro. To validate the specificity of the CARs, CRISPR/Cas9 was used to generate TM4SF1 knockouts (KO) in the UMUC3 cell line, which abolished the anti-tumor activity of TM4SF1-CAR T cells (FIG. 20).

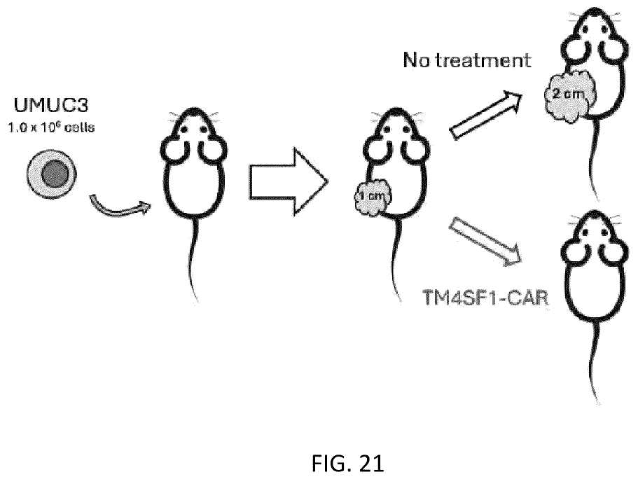

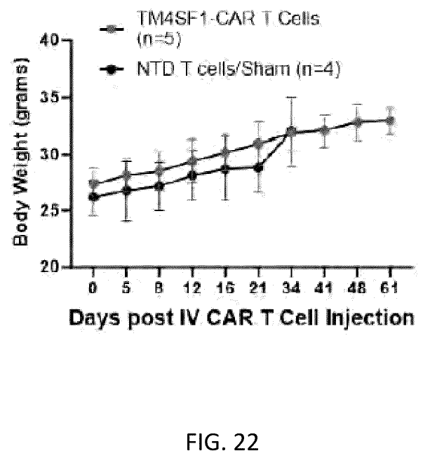

Finally, CAR1 was tested against xenografts derived from the UMUC3 cell line (FIG. 21), which was selected for its high TM4SF1 expression and negative absent NECTIN4 expression. CAR1 exhibited potent anti-tumor activity against these tumors in vivo. Whereas control mice all died by day 37, mice treated with TM4SF1-CAR1 cells had complete and durable responses, even up to day 100 (n=5 mice, FIG. 6D-E). Importantly, mice treated with TM4SF1-CAR1 cells had stable weights (FIG. 22) and no overt pulmonary toxicity. Taken together, these data demonstrate that TM4SF1 could be a new therapeutic target for HV bladder cancers, including tumors lacking NECTIN4 expression, and can be successfully targeted using CAR T cell therapy.

### 3. Discussion

scRNA-seq can be used to identify molecular features for rare, understudied cancer types such has HV bladder cancers. Here, a cancer cell state (Cluster 13) is described with clinical and mechanistic significance and a targetable protein (TM4SF1) in HV bladder cancers. As HVs are poorly understood in part because they are heterogenous and uncommon, scRNAseq enabled significant insights about HV cancer biology in a relatively small cohort of tumors. The study underscores the potential of scRNAseq technologies in precision cancer medicine.

The identification of a distinct “Cluster 13” cell state, which was found in more than half of the sequenced HV tumors and can be detected using MUC16 (CA125) as a marker, has potentially important clinical implications for HV bladder cancers. Since CA125+ cells are found in most HVs and are enriched in metastatic disease, a deeper characterization of this cell state may lead to new unified strategies to treat tumors that otherwise exhibit a great degree of heterogeneity. Although tumor cells harboring this cell state are predicted to be more resistant to conventional chemotherapeutics used for bladder cancer such as cisplatin, gemcitabine, doxorubicin, vinblastine, and mitomycin C, several United States Food and Drug Administration (FDA)-approved agents including omipalisib (PI3K/mTOR inhibitor), belinostat (histone deacetylase inhibitor), and quizartinib (FLT3 inhibitor) were predicted to be more effective against this group of cells (Cluster 13) compared to other tumor cells.

The specific expression of MUC16 (CA125) and other mucin genes in this cell state is intriguing. CA125, a well described gene more commonly associated with ovarian and pancreatic cancers, is a membrane-bound mucin protein that can promote cancer invasion and metastasis, and it has also been associated with therapeutic resistance in bladder cancer. Here it is shown that patients with HV tumors have higher serum CA125 levels compared to patients with UC tumors, indicating its use as a biomarker in bladder cancer and could be useful for serological monitoring of HV tumors.

The origin of the cancer cell state identified in Cluster 13 remains an important question. While the data indicate that Cluster 13 cells are a common state that is found in different HV tumors, the temporal relationship between Cluster 13 cells and non-Cluster 13 other cancer cells within each tumor cannot be determined using scRNA-seq alone. It remains possible that the Cluster 13 cell state represents a common precursor cell state for HV tumors. The existence of a common cell state that could be highly metastatic and chemotherapy-resistant among diverse HV tumors argues that a common mechanism may underlie their aggressive behavior.

HV tumors exhibit transcriptional programs characteristic of the non-urothelial cell types to which they share histologic resemblance. This raises the possibility that more HVs could be treated using agents targeting those other tumor cell types. Appropriating therapies designed for other cancers has been shown empirically to be effective in the case of SCBC and SCLC, and this provides evidence for how SCBC and SCLC can have overlapping transcriptional programs. The existence of plasma cell transcriptional programs is demonstrated in the plasmacytoid HV. This provides a rationale to test whether therapies designed for plasma cell neoplasms could be effective for this HV in future studies.

The discovery that HV tumor cells broadly express TM4SF1, a gene that encodes a surface protein that has already been implicated in the pathogenesis of aggressive bladder cancers and other cancer cell types, has therapeutic implications. TM4SF1 is a promising target because its expression is not limited to HV bladder cancers and its negative association with PVRL4/PRR4 (NECTIN4) expression suggests that targeted therapy against TM4SF1 could complement existing targeted agents. Antibody-mediated inhibition of TM4SF1 has been previously shown to have therapeutic potential against cancer stem cells in vitro; now it is demonstrated that durable anti-tumor responses in mice bearing xenografts with minimal toxicity.

In conclusion, the study demonstrates that HV subtypes in bladder cancer contain harbor a clinically significant CA125+ cell state, express a surface antigen that is targetable using CAR T cells, and share transcriptional features with histologically similar non-urothelial cancers.

Those skilled in the art will recognize, or be able to ascertain using no more than routine experimentation, many equivalents to the specific embodiments of the method and compositions described herein. Such equivalents are intended to be encompassed by the following claims.

| | |
|---|---|
| LOCUS | TM4SF1_01VHVL in NB-ready-CAR-bb 11303 bp DNA circular |
| | 22-JUN-2023 |
| DEFINITION | AGX01VHVL in NB-ready-CAR-bb |
| COMMENT | Resistance markers: Ampicillin |
| COMMENT | This file was created using tools provided by Twist |
| | Bioscience |
| FEATURES | Location/Qualifiers |
| source | 1..11303 |
| | /organism=″TM4SF1_01VHVL in NB-ready-CAR-bb″ |
| misc_feature_ | 1..747 |
| | /label=″insert″ |
| misc_feature_ | 748.. 11303 |
| | /label=″vector_backbone-747-11303″ |
| misc_feature_ | 1450..2166 |
| | /label=″EGFP″ |
| misc_feature_ | 2973..3025 |
| | /label=″delta_U3″ |
| misc_feature_ | 3233..3249 |
| | /label=″Sp6″ |
| misc_feature_ | 4037..4141 |
| | /label=″AmpR_promoter″ |
| misc_feature_ | 4142..5002 |
| | /label=″ampR″ |
| misc_feature_ | 5173..5761 |
| | /label=″colE1_high_copy″ |
| misc_feature_ | 5997..6233 |
| | /label=″SV40ER″ |
| misc_feature_ | 6020..6288 |
| | /label=″SV40″ |
| misc_feature_ | 7469..7484 |
| | /label=″SV40_int″ |
| misc_feature_ | 7480..7537 |
| | /label=″SV40_3_splice″ |
| misc_feature_ | 8004..8244 |
| | /label=″SV40_PA″ |
| misc_feature_ | 8412..9045 |
| | /label=″3_prime_LTR″ |
| misc_feature_ | 9092..9217 |
| | /label=″HIV-1_pack″ |
| misc_feature_ | 9156..9200 |
| | /label=″HIV-1_psi_pack″ |
| misc_feature_ | 9714..9947 |
| | /label=″RRE″ |

ORIGIN

| | |
|---|---|
| 1 | GAGGTGATCC TGGTCGAGAG TGGAGGTGGG TTGGTTAAGC CCGGAGGTAG CCTGAAGTTA |
| 61 | TCCTGTGCCG CCAGCGGCTT TACTTTCAGT TCTTTCGCTA TGTCCTGGGT GCGCCAAACG |
| 121 | CCGGAAAAGC GGCTGGAGTG GGTGGCTACC ATCTCATCCG GGAGCATTTA TATATATTAT |
| 181 | ACAGATGGTG TGAAAGGCCG ATTTACCATC AGCCGGGACA ACGCGAAGAA CACCGTCCAC |
| 241 | CTGCAAATGT CTTCTTTACG ATCAGAAGAC ACAGCCATGT ACTACTGTGC TAGGCGAGGA |
| 301 | ATCTATTATG GCTATGATGG GTACGCCATG GATTATTGGG GCCAGGGCAC CAGTGTGACC |
| 361 | GTCTCAGGCG GCGGCGGATC CGGAGGAGGA GGCAGCGGGG GCGGCGGTTC CGCTGTGGTG |
| 421 | ATGACTCAAA CGCCCCTCTC CCTGCCTGTC TCACTCGGGG ACCAGGCTTC CATTAGTTGC |
| 481 | AGGAGCAGCC AGTCTTTAGT GCACTCCAAC GGCAACACAT ATCTGCATTG GTATATGCAA |
| 541 | AAACCAGGAC AATCCCCTAA AGTGCTCATT TACAAAGTGT CAAACCGCTT CAGCGGAGTG |
| 601 | CCCGACAGAT TTTCCGGCTC AGGCTCAGGG ACCGACTTCA CTTTAAAGAT TTCCAGAGTG |
| 661 | GAAGCCGACG ATCTGGGGAT CTACTTCTGC TCCCAAAGCA CTCATATTCC CCTGGCCTTC |
| 721 | GGAGCTGGAA CTAAACTGGA ACTCAAAACC ACGACGCCAG CGCCGCGACC ACCAACACCG |
| 781 | GCGCCCACCA TCGCGTCGCA GCCCCTGTCC CTGCGCCCAG AGGCGTGCCG GCCAGCGGCG |
| 841 | GGGGGCGCAG TGCACACGAG GGGGCTGGAC TTCGCCTGTG ATATCTACAT CTGGGCGCCC |
| 901 | TTGGCCGGGA CTTGTGGGGT CCTTCTCCTG TCACTGGTTA TCACCCTTTA CTGCTCCCTA |
| 961 | AAACGGGGCA GAAAGAAACT CCTGTATATA TTCAAACAAC CATTTATGAG ACCAGTACAA |
| 1021 | ACTACTCAAG AGGAAGATGG CTGTAGCTGC CGATTTCCAG AAGAAGAAGA AGGAGGATGT |
| 1081 | GAACTGAGAG TGAAGTTCAG CAGGAGCGCA GACGCCCCCG CGTACAAGCA GGGCCAGAAC |
| 1141 | CAGCTCTATA ACGAGCTCAA TCTAGGACGA AGAGAGGAGT ACGATGTTTT GGACAAGAGA |
| 1201 | CGTGGCCGGG ACCCTGAGAT GGGGGGAAAG CCGAGAAGGA AGAACCCTCA GGAAGGCCTG |
| 1261 | TACAATGAAC TGCAGAAAGA TAAGATGGCG GAGGCCTACA GTGAGATTGG GATGAAAGGC |
| 1321 | GAGCGCCGGA GGGGCAAGGG GCACGATGGC CTTTACCAGG GTCTCAGTAC AGCCACCAAG |
| 1381 | GACACCTACG ACGCCCTTCA CATGCAGGCC CTGCCTCCTC GCTCGGGAAG CGGGTCCGGT |
| 1441 | AGCGGATCTA TGGTGAGCAA GGGCGAGGAG CTGTTCACCG GGGTGGTGCC CATCCTGGTC |
| 1501 | GAGCTGGACG GCGACGTAAA CGGCCACAAG TTCAGCGTGT CCGGCGAGGG CGAGGGCGAT |
| 1561 | GCCACCTACG GCAAGCTGAC CCTGAAGTTC ATCTGCACCA CCGGCAAGCT GCCCGTGCCC |
| 1621 | TGGCCCACCC TCGTGACCAC CCTGACCTAC GGCGTGCAGT GCTTCAGCCG CTACCCCGAC |
| 1681 | CACATGAAGC AGCACGACTT CTTCAAGTCC GCCATGCCCG AAGGCTACGT CCAGGAGCGC |
| 1741 | ACCATCTTCT TCAAGGACGA CGGCAACTAC AAGACCCGCG CCGAGGTGAA GTTCGAGGGC |
| 1801 | GACACCCTGG TGAACCGCAT CGAGCTGAAG GGCATCGACT TCAAGGAGGA CGGCAACATC |
| 1861 | CTGGGGCACA AGCTGGAGTA CAACTACAAC AGCCACAACG TCTATATCAT GGCCGACAAG |
| 1921 | CAGAAGAACG GCATCAAGGT GAACTTCAAG ATCCGCCACA ACATCGAGGA CGGCAGCGTG |
| 1981 | CAGCTCGCCG ACCACTACCA GCAGAACACC CCCATCGGCG ACGGCCCCGT GCTGCTGCCC |
| 2041 | GACAACCACT ACCTGAGCAC CCAGTCCGCC CTGAGCAAAG ACCCCAACGA GAAGCGCGAT |
| 2101 | CACATGGTCC TGCTGGAGTT CGTGACCGCC GCCGGGATCA CTCTCGGCAT GGACGAGCTG |
| 2161 | TACAAGTAAC TTGACTTGCG GCCGCAACTC CCACCTGCAA CATGCGTGAC TGACTGAGGC |
| 2221 | CGCGACTCTA GAGTCGACCT GCAGGCATGC AAGCTTGATA TCAAGCTTAT CGATAATCAA |
| 2281 | CCTCTGGATT ACAAAATTTG TGAAAGATTG ACTGGTATTC TTAACTATGT TGCTCCTTTT |
| 2341 | ACGCTATGTG GATACGCTGC TTTAATGCCT TTGTATCATG CTATTGCTTC CCGTATGGCT |
| 2401 | TTCATTTTCT CCTCCTTGTA TAAATCCTGG TTGCTGTCTC TTTATGAGGA GTTGTGGCCC |
| 2461 | GTTGTCAGGC AACGTGGCGT GGTGTGCACT GTGTTTGCTG ACGCAACCCC CACTGGTTGG |
| 2521 | GGCATTGCCA CCACCTGTCA GCTCCTTTCC GGGACTTTCG CTTTCCCCCT CCCTATTGCC |
| 2581 | ACGGCGGAAC TCATCGCCGC CTGCCTTGCC CGCTGCTGGA CAGGGGCTCG GCTGTTGGGC |
| 2641 | ACTGACAATT CCGTGGTGTT GTCGGGGAAA TCATCGTCCT TTCCTTGGCT GCTCGCCTGT |
| 2701 | GTTGCCACCT GGATTCTGCG CGGGACGTCC TTCTGCTACG TCCCTTCGGC CCTCAATCCA |
| 2761 | GCGGACCTTC CTTCCCGCGG CCTGCTGCCG GCTCTGCGGC CTCTTCCGCG TCTTCGCCTT |
| 2821 | CGCCCTCAGA CGAGTCGGAT CTCCCTTTGG GCCGCCTCCC CGCATCGATA CCGTCGACCT |
| 2881 | CGAGGGAATT AATTCGAGCT CGGTACCTTT AAGACCAATG ACTTACAAGG CAGCTGTAGA |
| 2941 | TCTTAGCCAC TTTTTAAAAG AAAAGGGGGG ACTGGAAGGG CTAATTCACT CCCAACGAAG |
| 3001 | ACAAGATCTG CTTTTTGCTT GTACTGGGTC TCTCTGGTTA GACCAGATCT GAGCCTGGGA |
| 3061 | GCTCTCTGGC TAACTAGGGA ACCCACTGCT TAAGCCTCAA TAAAGCTTGC CTTGAGTGCT |
| 3121 | TCAAGTAGTG TGTGCCCGTC TGTTGTGTGA CTCTGGTAAC TAGAGATCCC TCAGACCCTT |
| 3181 | TTAGTCAGTG TGGAAAATCT CTAGCAGCAT CTAGAATTAA TTCCGTGTAT TCTATAGTGT |
| 3241 | CACCTAAATC GTATGTGTAT GATACATAAG GTTATGTATT AATTGTAGCC GCGTTCTAAC |
| 3301 | GACAATATGT ACAAGCCTAA TTGTGTAGCA TCTGGCTTAC TGAAGCAGAC CCTATCATCT |
| 3361 | CTCTCGTAAA CTGCCGTCAG AGTCGGTTTG GTTGGACGAA CCTTCTGAGT TTCTGGTAAC |
| 3421 | GCCGTCCCGC ACCCGGAAAT GGTCAGCGAA CCAATCAGCA GGGTCATCGC TAGCCAGATC |
| 3481 | CTCTACGCCG GACGCATCGT GGCCGGCATC ACCGGCGCCA CAGGTGCGGT TGCTGGCGCC |
| 3541 | TATATCGCCG ACATCACCGA TGGGGAAGAT CGGGCTCGCC ACTTCGGGCT CATGAGCGCT |
| 3601 | TGTTTCGGCG TGGGTATGGT GGCAGGCCCC GTGGCCGGGG GACTGTTGGG CGCCATCTCC |
| 3661 | TTGCATGCAC CATTCCTTGC GGCGGCGGTG CTCAACGGCC TCAACCTACT ACTGGGCTGC |
| 3721 | TTCCTAATGC AGGAGTCGCA TAAGGGAGAG CGTCGAATGG TGCACTCTCA GTACAATCTG |
| 3781 | CTCTGATGCC GCATAGTTAA GCCAGCCCCG ACACCCGCCA ACACCCGCTG ACGCGCCCTG |
| 3841 | ACGGGCTTGT CTGCTCCCGG CATCCGCTTA CAGACAAGCT GTGACCGTCT CCGGGAGCTG |
| 3901 | CATGTGTCAG AGGTTTTCAC CGTCATCACC GAAACGCGCG AGACGAAAGG GCCTCGTGAT |
| 3961 | ACGCCTATTT TTATAGGTTA ATGTCATGAT AATAATGGTT TCTTAGACGT CAGGTGGCAC |
| 4021 | TTTTCGGGGA AATGTGCGCG GAACCCCTAT TTGTTTATTT TTCTAAATAC ATTCAAATAT |
| 4081 | GTATCCGCTC ATGAGACAAT AACCCTGATA AATGCTTCAA TAATATTGAA AAAGGAAGAG |
| 4141 | TATGAGTATT CAACATTTCC GTGTCGCCCT TATTCCCTTT TTTGCGGCAT TTTGCCTTCC |
| 4201 | TGTTTTTGCT CACCCAGAAA CGCTGGTGAA AGTAAAAGAT GCTGAAGATC AGTTGGGTGC |
| 4261 | ACGAGTGGGT TACATCGAAC TGGATCTCAA CAGCGGTAAG ATCCTTGAGA GTTTTCGCCC |
| 4321 | CGAAGAACGT TTTCCAATGA TGAGCACTTT TAAAGTTCTG CTATGTGGCG CGGTATTATC |
| 4381 | CCGTATTGAC GCCGGGCAAG AGCAACTCGG TCGCCGCATA CACTATTCTC AGAATGACTT |
| 4441 | GGTTGAGTAC TCACCAGTCA CAGAAAAGCA TCTTACGGAT GGCATGACAG TAAGAGAATT |
| 4501 | ATGCAGTGCT GCCATAACCA TGAGTGATAA CACTGCGGCC AACTTACTTC TGACAACGAT |
| 4561 | CGGAGGACCG AAGGAGCTAA CCGCTTTTTT GCACAACATG GGGGATCATG TAACTCGCCT |
| 4621 | TGATCGTTGG GAACCGGAGC TGAATGAAGC CATACCAAAC GACGAGCGTG ACACCACGAT |
| 4681 | GCCTGTAGCA ATGGCAACAA CGTTGCGCAA ACTATTAACT GGCGAACTAC TTACTCTAGC |
| 4741 | TTCCCGGCAA CAATTAATAG ACTGGATGGA GGCGGATAAA GTTGCAGGAC CACTTCTGCG |
| 4801 | CTCGGCCCTT CCGGCTGGCT GGTTTATTGC TGATAAATCT GGAGCCGGTG AGCGTGGGTC |
| 4861 | TCGCGGTATC ATTGCAGCAC TGGGGCCAGA TGGTAAGCCC TCCCGTATCG TAGTTATCTA |
| 4921 | CACGACGGGG AGTCAGGCAA CTATGGATGA ACGAAATAGA CAGATCGCTG AGATAGGTGC |
| 4981 | CTCACTGATT AAGCATTGGT AACTGTCAGA CCAAGTTTAC TCATATATAC TTTAGATTGA |
| 5041 | TTTAAAACTT CATTTTTAAT TTAAAAGGAT CTAGGTGAAG ATCCTTTTTG ATAATCTCAT |
| 5101 | GACCAAAATC CCTTAACGTG AGTTTTCGTT CCACTGAGCG TCAGACCCCG TAGAAAAGAT |
| 5161 | CAAAGGATCT TCTTGAGATC CTTTTTTTCT GCGCGTAATC TGCTGCTTGC AAACAAAAAA |
| 5221 | ACCACCGCTA CCAGCGGTGG TTTGTTTGCC GGATCAAGAG CTACCAACTC TTTTTCCGAA |
| 5281 | GGTAACTGGC TTCAGCAGAG CGCAGATACC AAATACTGTT CTTCTAGTGT AGCCGTAGTT |
| 5341 | AGGCCACCAC TTCAAGAACT CTGTAGCACC GCCTACATAC CTCGCTCTGC TAATCCTGTT |
| 5401 | ACCAGTGGCT GCTGCCAGTG GCGATAAGTC GTGTCTTACC GGGTTGGACT CAAGACGATA |
| 5461 | GTTACCGGAT AAGGCGCAGC GGTCGGGCTG AACGGGGGGT TCGTGCACAC AGCCCAGCTT |
| 5521 | GGAGCGAACG ACCTACACCG AACTGAGATA CCTACAGCGT GAGCTATGAG AAAGCGCCAC |
| 5581 | GCTTCCCGAA GGGAGAAAGG CGGACAGGTA TCCGGTAAGC GGCAGGGTCG GAACAGGAGA |
| 5641 | GCGCACGAGG GAGCTTCCAG GGGGAAACGC CTGGTATCTT TATAGTCCTG TCGGGTTTCG |
| 5701 | CCACCTCTGA CTTGAGCGTC GATTTTTGTG ATGCTCGTCA GGGGGGCGGA GCCTATGGAA |
| 5761 | AAACGCCAGC AACGCGGCCT TTTTACGGTT CCTGGCCTTT TGCTGGCCTT TTGCTCACAT |
| 5821 | GTTCTTTCCT GCGTTATCCC CTGATTCTGT GGATAACCGT ATTACCGCCT TTGAGTGAGC |
| 5881 | TGATACCGCT CGCCGCAGCC GAACGACCGA GCGCAGCGAG TCAGTGAGCG AGGAAGCGGA |
| 5941 | AGAGCGCCCA ATACGCAAAC CGCCTCTCCC CGCGCGTTGG CCGATTCATT AATGCAGCTG |
| 6001 | TGGAATGTGT GTCAGTTAGG GTGTGGAAAG TCCCCAGGCT CCCCAGCAGG CAGAAGTATG |
| 6061 | CAAAGCATGC ATCTCAATTA GTCAGCAACC AGGTGTGGAA AGTCCCCAGG CTCCCCAGCA |
| 6121 | GGCAGAAGTA TGCAAAGCAT GCATCTCAAT TAGTCAGCAA CCATAGTCCC GCCCCTAACT |
| 6181 | CCGCCCATCC CGCCCCTAAC TCCGCCCAGT TCCGCCCATT CTCCGCCCCA TGGCTGACTA |
| 6241 | ATTTTTTTTA TTTATGCAGA GGCCGAGGCC GCCTCGGCCT CTGAGCTATT CCAGAAGTAG |
| 6301 | TGAGGAGGCT TTTTTGGAGG CCTAGGCTTT TGCAAAAAGC TTGGACACAA GACAGGCTTG |
| 6361 | CGAGATATGT TTGAGAATAC CACTTTATCC CGCGTCAGGG AGAGGCAGTG CGTAAAAAGA |
| 6421 | CGCGGACTCA TGTGAAATAC TGGTTTTTAG TGCGCCAGAT CTCTATAATC TCGCGCAACC |
| 6481 | TATTTTCCCC TCGAACACTT TTTAAGCCGT AGATAAACAG GCTGGGACAC TTCACATGAG |
| 6541 | CGAAAAATAC ATCGTCACCT GGGACATGTT GCAGATCCAT GCACGTAAAC TCGCAAGCCG |
| 6601 | ACTGATGCCT TCTGAACAAT GGAAAGGCAT TATTGCCGTA AGCCGTGGCG GTCTGTACCG |
| 6661 | GGTGCGTTAC TGGCGCGTGA ACTGGGTATT CGTCATGTCG ATACCGTTTG TATTTCCAGC |
| 6721 | TACGATCACG ACAACCAGCG CGAGCTTAAA GTGCTGAAAC GCGCAGAAGG CGATGGCGAA |
| 6781 | GGCTTCATCG TTATTGATGA CCTGGTGGAT ACCGGTGGTA CTGCGGTTGC GATTCGTGAA |
| 6841 | ATGTATCCAA AAGCGCACTT TGTCACCATC TTCGCAAAAC CGGCTGGTCG TCCGCTGGTT |
| 6901 | GATGACTATG TTGTTGATAT CCCGCAAGAT ACCTGGATTG AACAGCCGTG GGATATGGGC |
| 6961 | GTCGTATTCG TCCCGCCAAT CTCCGGTCGC TAATCTTTTC AACGCCTGGC ACTGCCGGGC |
| 7021 | GTTGTTCTTT TTAACTTCAG GCGGGTTACA ATAGTTTCCA GTAAGTATTC TGGAGGCTGC |
| 7081 | ATCCATGACA CAGGCAAACC TGAGCGAAAC CCTGTTCAAA CCCCGCTTTA AACATCCTGA |
| 7141 | AACCTCGACG CTAGTCCGCC GCTTTAATCA CGGCGCACAA CCGCCTGTGC AGTCGGCCCT |
| 7201 | TGATGGTAAA ACCATCCCTC ACTGGTATCG CATGATTAAC CGTCTGATGT GGATCTGGCG |
| 7261 | CGGCATTGAC CCACGCGAAA TCCTCGACGT CCAGGCACGT ATTGTGATGA GCGATGCCGA |
| 7321 | ACGTACCGAC GATGATTTAT ACGATACGGT GATTGGCTAC CGTGGCGGCA ACTGGATTTA |
| 7381 | TGAGTGGGCC CCGGATCTTT GTGAAGGAAC CTTACTTCTG TGGTGTGACA TAATTGGACA |
| 7441 | AACTACCTAC AGAGATTTAA AGCTCTAAGG TAAATATAAA ATTTTTAAGT GTATAATGTG |
| 7501 | TTAAACTACT GATTCTAATT GTTTGTGTAT TTTAGATTCC AACCTATGGA ACTGATGAAT |
| 7561 | GGGAGCAGTG GTGGAATGCC TTTAATGAGG AAAACCTGTT TTGCTCAGAA GAAATGCCAT |
| 7621 | CTAGTGATGA TGAGGCTACT GCTGACTCTC AACATTCTAC TCCTCCAAAA AAGAAGAGAA |
| 7681 | AGGTAGAAGA CCCCAAGGAC TTTCCTTCAG AATTGCTAAG TTTTTTGAGT CATGCTGTGT |
| 7741 | TTAGTAATAG AACTCTTGCT TGCTTTGCTA TTTACACCAC AAAGGAAAAA GCTGCACTGC |
| 7801 | TATACAAGAA AATTATGGAA AAATATTCTG TAACCTTTAT AAGTAGGCAT AACAGTTATA |
| 7861 | ATCATAACAT ACTGTTTTTT CTTACTCCAC ACAGGCATAG AGTGTCTGCT ATTAATAACT |
| 7921 | ATGCTCAAAA ATTGTGTACC TTTAGCTTTT TAATTTGTAA AGGGGTTAAT AAGGAATATT |
| 7981 | TGATGTATAG TGCCTTGACT AGAGATCATA ATCAGCCATA CCACATTTGT AGAGGTTTTA |
| 8041 | CTTGCTTTAA AAAACCTCCC ACACCTCCCC CTGAACCTGA AACATAAAAT GAATGCAATT |
| 8101 | GTTGTTGTTA ACTTGTTTAT TGCAGCTTAT AATGGTTACA AATAAAGCAA TAGCATCACA |
| 8161 | AATTTCACAA ATAAAGCATT TTTTTCACTG CATTCTAGTT GTGGTTTGTC CAAACTCATC |
| 8221 | AATGTATCTT ATCATGTCTG GATCAACTGG ATAACTCAAG CTAACCAAAA TCATCCCAAA |
| 8281 | CTTCCCACCC CATACCCTAT TACCACTGCC AATTACCTGT GGTTTCATTT ACTCTAAACC |
| 8341 | TGTGATTCCT CTGAATTATT TTCATTTTAA AGAAATTGTA TTTGTTAAAT ATGTACTACA |
| 8401 | AACTTAGTAG TTGGAAGGGC TAATTCACTC CCAAAGAAGA CAAGATATCC TTGATCTGTG |
| 8461 | GATCTACCAC ACACAAGGCT ACTTCCCTGA TTAGCAGAAC TACACACCAG GGCCAGGGGT |
| 8521 | CAGATATCCA CTGACCTTTG GATGGTGCTA CAAGCTAGTA CCAGTTGAGC CAGATAAGGT |
| 8581 | AGAAGAGGCC AATAAAGGAG AGAACACCAG CTTGTTACAC CCTGTGAGCC TGCATGGGAT |
| 8641 | GGATGACCCG GAGAGAGAAG TGTTAGAGTG GAGGTTTGAC AGCCGCCTAG CATTTCATCA |
| 8701 | CGTGGCCCGA GAGCTGCATC CGGAGTACTT CAAGAACTGC TGATATCGAG CTTGCTACAA |
| 8761 | GGGACTTTCC GCTGGGGACT TTCCAGGGAG GCGTGGCCTG GGCGGGACTG GGGAGTGGCG |
| 8821 | AGCCCTCAGA TCCTGCATAT AAGCAGCTGC TTTTTGCCTG TACTGGGTCT CTCTGGTTAG |
| 8881 | ACCAGATCTG AGCCTGGGAG CTCTCTGGCT AACTAGGGAA CCCACTGCTT AAGCCTCAAT |
| 8941 | AAAGCTTGCC TTGAGTGCTT CAAGTAGTGT GTGCCCGTCT GTTGTGTGAC TCTGGTAACT |
| 9001 | AGAGATCCCT CAGACCCTTT TAGTCAGTGT GGAAAATCTC TAGCAGTGGC GCCCGAACAG |
| 9061 | GGACTTGAAA GCGAAAGGGA AACCAGAGGA GCTCTCTCGA CGCAGGACTC GGCTTGCTGA |
| 9121 | AGCGCGCACG GCAAGAGGCG AGGGGCGGCG ACTGGTGAGT ACGCCAAAAA TTTTGACTAG |
| 9181 | CGGAGGCTAG AAGGAGAGAG ATGGGTGCGA GAGCGTCAGT ATTAAGCGGG GGAGAATTAG |
| 9241 | ATCGCGATGG GAAAAAATTC GGTTAAGGCC AGGGGGAAAG AAAAAATATA AATTAAAACA |
| 9301 | TATAGTATGG GCAAGCAGGG AGCTAGAACG ATTCGCAGTT AATCCTGGCC TGTTAGAAAC |
| 9361 | ATCAGAAGGC TGTAGACAAA TACTGGGACA GCTACAACCA TCCCTTCAGA CAGGATCAGA |
| 9421 | AGAACTTAGA TCATTATATA ATACAGTAGC AACCCTCTAT TGTGTGCATC AAAGGATAGA |
| 9481 | GATAAAAGAC ACCAAGGAAG CTTTAGACAA GATAGAGGAA GAGCAAAACA AAAGTAAGAC |
| 9541 | CACCGCACAG CAAGCGGCCG GCCGCTGATC TTCAGACCTG GAGGAGGAGA TATGAGGGAC |
| 9601 | AATTGGAGAA GTGAATTATA TAAATATAAA GTAGTAAAAA TTGAACCATT AGGAGTAGCA |
| 9661 | CCCACCAAGG CAAAGAGAAG AGTGGTGCAG AGAGAAAAAA GAGCAGTGGG AATAGGAGCT |
| 9721 | TTGTTCCTTG GGTTCTTGGG AGCAGCAGGA AGCACTATGG GCGCAGCGTC AATGACGCTG |
| 9781 | ACGGTACAGG CCAGACAATT ATTGTCTGGT ATAGTGCAGC AGCAGAACAA TTTGCTGAGG |
| 9841 | GCTATTGAGG CGCAACAGCA TCTGTTGCAA CTCACAGTCT GGGGCATCAA GCAGCTCCAG |
| 9901 | GCAAGAATCC TGGCTGTGGA AAGATACCTA AAGGATCAAC AGCTCCTGGG GATTTGGGGT |
| 9961 | TGCTCTGGAA AACTCATTTG CACCACTGCT GTGCCTTGGA ATGCTAGTTG GAGTAATAAA |
| 10021 | TCTCTGGAAC AGATTTGGAA TCACACGACC TGGATGGAGT GGGACAGAGA AATTAACAAT |
| 10081 | TACACAAGCT TAATACACTC CTTAATTGAA GAATCGCAAA ACCAGCAAGA AAAGAATGAA |
| 10141 | CAAGAATTAT TGGAATTAGA TAAATGGGCA AGTTTGTGGA ATTGGTTTAA CATAACAAAT |
| 10201 | TGGCTGTGGT ATATAAAATT ATTCATAATG ATAGTAGGAG GCTTGGTAGG TTTAAGAATA |
| 10261 | GTTTTTGCTG TACTTTCTAT AGTGAATAGA GTTAGGCAGG GATATTCACC ATTATCGTTT |
| 10321 | CAGACCCACC TCCCAACCCC GAGGGGACCC GACAGGCCCG AAGGAATAGA AGAAGAAGGT |
| 10381 | GGAGAGAGAG ACAGAGACAG ATCCATTCGA TTAGTGAACG GATCTCGACG GTATCGCCAA |
| 10441 | ATGGCAGTAT TCATCCACAA TTTTAAAAGA AAAGGGGGGA TTGGGGGGTA CAGTGCAGGG |
| 10501 | GAAAGAATAG TAGACATAAT AGCAACAGAC ATACAAACTA AAGAATTACA AAAACAAATT |
| 10561 | ACAAAAATTC AAAATTTTCG GGTTTATTAC AGGGACAGCA GAGATCCAGT TTGGATCGAT |
| 10621 | AAGCTTGATA TCGAATTCCT GCAGCCCCGA TAAAATAAAA GATTTTATTT AGTCTCCAGA |
| 10681 | AAAAGGGGGG AATGAAAGAC CCCACCTGTA GGTTTGGCAA GCTAGCTGCA GTAACGCCAT |
| 10741 | TTTGCAAGGC ATGGAAAAAT ACCAAACCAA GAATAGAGAA GTTCAGATCA AGGGCGGGTA |
| 10801 | CATGAAAATA GCTAACGTTG GGCCAAACAG GATATCTGCG GTGAGCAGTT TCGGCCCCGG |
| 10861 | CCCGGGGCCA AGAACAGATG GTCACCGCAG TTTCGGCCCC GGCCCGAGGC CAAGAACAGA |
| 10921 | TGGTCCCCAG ATATGGCCCA ACCCTCAGCA GTTTCTTAAG ACCCATCAGA TGTTTCCAGG |
| 10981 | CTCCCCCAAG GACCTGAAAT GACCCTGCGC CTTATTTGAA TTAACCAATC AGCCTGCTTC |
| 11041 | TCGCTTCTGT TCGCGCGCTT CTGCTTCCCG AGCTCTATAA AAGAGCTCAC AACCCCTCAC |
| 11101 | TCGGCGCGCC AGTCCTCCGA CAGACTGAGT CGCCCGGGGG GGATCTGGAG CTCTCGAGAA |
| 11161 | TTCTCACGCG TCAAGTGGAG CAAGGCAGGT GGACAGTGAT GGCCTTACCA GTGACCGCCT |
| 11221 | TGCTCCTGCC GCTGGCCTTG CTGCTCCACG CCGCCAGGCC GGAGCAGAAG CTGATCAGCG |
| 11281 | AGGAGGACCT GGAGGAGGAC CTG |

(SEQ ID NO: 128)
//

| | |
|---|---|
| LOCUS | TM4SF1_01VLVH in NB-ready-CAR-bb 11303 bp DNA circular 22- |
| | JUN-2023 |
| DEFINITION | AGX01VLVH in NB-ready-CAR-bb |
| COMMENT | Resistance markers: Ampicillin |
| COMMENT | This file was created using tools provided by Twist |
| | Bioscience |
| FEATURES | Location/Qualifiers |
| source | 1..11303 |
| | /organism=″TM4SF1_01VLVH in NB-ready-CAR-bb″ |
| misc_feature_ | 1..747 |
| | /label=″insert″ |
| misc_feature_ | 748..11303 |
| | /label=″vector_backbone-747-11303″ |
| misc_feature_ | 1450..2166 |
| | /label=″EGFP″ |
| misc_feature_ | 2973..3025 |
| | /label=″delta_U3″ |
| misc_feature_ | 3233..3249 |
| | /label=″Sp6″ |
| misc_feature_ | 4037..4141 |
| | /label=″AmpR_promoter″ |
| misc_feature_ | 4142..5002 |
| | /label=″ampR″ |
| misc_feature_ | 5173..5761 |
| | /label=″colE1_high_copy″ |
| misc_feature_ | 5997..6233 |
| | /label=″SV40ER″ |
| misc_feature_ | 6020..6288 |
| | /label=″SV40″ |
| misc_feature_ | 7469..7484 |
| | /label=″SV40_int″ |
| misc_feature_ | 7480..7537 |
| | /label=″SV40_3_splice″ |
| misc_feature_ | 8004..8244 |
| | /label=″SV40_PA″ |
| misc_feature_ | 8412..9045 |
| | /label=″3_prime_LTR″ |
| misc_feature_ | 9092..9217 |
| | /label=″HIV-1_pack″ |
| misc_feature_ | 9156..9200 |
| | /label=″HIV-1_psi_pack″ |
| misc_feature_ | 9714..9947 |
| | /label=″RRE″ |

ORIGIN

| | |
|---|---|
| 1 | GAGGTGATCC TGGTCGAGAG TGGAGGTGGG TTGGTTAAGC CCGGAGGTAG CCTGAAGTTA |
| 61 | TCCTGTGCCG CCAGCGGCTT TACTTTCAGT TCTTTCGCTA TGTCCTGGGT GCGCCAAACG |
| 121 | CCGGAAAAGC GGCTGGAGTG GGTGGCTACC ATCTCATCCG GGAGCATTTA TATATATTAT |
| 181 | ACAGATGGTG TGAAAGGCCG ATTTACCATC AGCCGGGACA ACGCGAAGAA CACCGTCCAC |
| 241 | CTGCAAATGT CTTCTTTACG ATCAGAAGAC ACAGCCATGT ACTACTGTGC TAGGCGAGGA |
| 301 | ATCTATTATG GCTATGATGG GTACGCCATG GATTATTGGG GCCAGGGCAC CAGTGTGACC |
| 361 | GTCTCAGGCG GCGGCGGATC CGGAGGAGGA GGCAGCGGGG GCGGCGGTTC CGCTGTGGTG |
| 421 | ATGACTCAAA CGCCCCTCTC CCTGCCTGTC TCACTCGGGG ACCAGGCTTC CATTAGTTGC |
| 481 | AGGAGCAGCC AGTCTTTAGT GCACTCCAAC GGCAACACAT ATCTGCATTG GTATATGCAA |
| 541 | AAACCAGGAC AATCCCCTAA AGTGCTCATT TACAAAGTGT CAAACCGCTT CAGCGGAGTG |
| 601 | CCCGACAGAT TTTCCGGCTC AGGCTCAGGG ACCGACTTCA CTTTAAAGAT TTCCAGAGTG |
| 661 | GAAGCCGACG ATCTGGGGAT CTACTTCTGC TCCCAAAGCA CTCATATTCC CCTGGCCTTC |
| 721 | GGAGCTGGAA CTAAACTGGA ACTCAAAACC ACGACGCCAG CGCCGCGACC ACCAACACCG |
| 781 | GCGCCCACCA TCGCGTCGCA GCCCCTGTCC CTGCGCCCAG AGGCGTGCCG GCCAGCGGCG |
| 841 | GGGGGCGCAG TGCACACGAG GGGGCTGGAC TTCGCCTGTG ATATCTACAT CTGGGCGCCC |
| 901 | TTGGCCGGGA CTTGTGGGGT CCTTCTCCTG TCACTGGTTA TCACCCTTTA CTGCTCCCTA |
| 961 | AAACGGGGCA GAAAGAAACT CCTGTATATA TTCAAACAAC CATTTATGAG ACCAGTACAA |
| 1021 | ACTACTCAAG AGGAAGATGG CTGTAGCTGC CGATTTCCAG AAGAAGAAGA AGGAGGATGT |
| 1081 | GAACTGAGAG TGAAGTTCAG CAGGAGCGCA GACGCCCCCG CGTACAAGCA GGGCCAGAAC |
| 1141 | CAGCTCTATA ACGAGCTCAA TCTAGGACGA AGAGAGGAGT ACGATGTTTT GGACAAGAGA |
| 1201 | CGTGGCCGGG ACCCTGAGAT GGGGGGAAAG CCGAGAAGGA AGAACCCTCA GGAAGGCCTG |
| 1261 | TACAATGAAC TGCAGAAAGA TAAGATGGCG GAGGCCTACA GTGAGATTGG GATGAAAGGC |
| 1321 | GAGCGCCGGA GGGGCAAGGG GCACGATGGC CTTTACCAGG GTCTCAGTAC AGCCACCAAG |
| 1381 | GACACCTACG ACGCCCTTCA CATGCAGGCC CTGCCTCCTC GCTCGGGAAG CGGGTCCGGT |
| 1441 | AGCGGATCTA TGGTGAGCAA GGGCGAGGAG CTGTTCACCG GGGTGGTGCC CATCCTGGTC |
| 1501 | GAGCTGGACG GCGACGTAAA CGGCCACAAG TTCAGCGTGT CCGGCGAGGG CGAGGGCGAT |
| 1561 | GCCACCTACG GCAAGCTGAC CCTGAAGTTC ATCTGCACCA CCGGCAAGCT GCCCGTGCCC |
| 1621 | TGGCCCACCC TCGTGACCAC CCTGACCTAC GGCGTGCAGT GCTTCAGCCG CTACCCCGAC |
| 1681 | CACATGAAGC AGCACGACTT CTTCAAGTCC GCCATGCCCG AAGGCTACGT CCAGGAGCGC |
| 1741 | ACCATCTTCT TCAAGGACGA CGGCAACTAC AAGACCCGCG CCGAGGTGAA GTTCGAGGGC |
| 1801 | GACACCCTGG TGAACCGCAT CGAGCTGAAG GGCATCGACT TCAAGGAGGA CGGCAACATC |
| 1861 | CTGGGGCACA AGCTGGAGTA CAACTACAAC AGCCACAACG TCTATATCAT GGCCGACAAG |
| 1921 | CAGAAGAACG GCATCAAGGT GAACTTCAAG ATCCGCCACA ACATCGAGGA CGGCAGCGTG |
| 1981 | CAGCTCGCCG ACCACTACCA GCAGAACACC CCCATCGGCG ACGGCCCCGT GCTGCTGCCC |
| 2041 | GACAACCACT ACCTGAGCAC CCAGTCCGCC CTGAGCAAAG ACCCCAACGA GAAGCGCGAT |
| 2101 | CACATGGTCC TGCTGGAGTT CGTGACCGCC GCCGGGATCA CTCTCGGCAT GGACGAGCTG |
| 2161 | TACAAGTAAC TTGACTTGCG GCCGCAACTC CCACCTGCAA CATGCGTGAC TGACTGAGGC |
| 2221 | CGCGACTCTA GAGTCGACCT GCAGGCATGC AAGCTTGATA TCAAGCTTAT CGATAATCAA |
| 2281 | CCTCTGGATT ACAAAATTTG TGAAAGATTG ACTGGTATTC TTAACTATGT TGCTCCTTTT |
| 2341 | ACGCTATGTG GATACGCTGC TTTAATGCCT TTGTATCATG CTATTGCTTC CCGTATGGCT |
| 2401 | TTCATTTTCT CCTCCTTGTA TAAATCCTGG TTGCTGTCTC TTTATGAGGA GTTGTGGCCC |
| 2461 | GTTGTCAGGC AACGTGGCGT GGTGTGCACT GTGTTTGCTG ACGCAACCCC CACTGGTTGG |
| 2521 | GGCATTGCCA CCACCTGTCA GCTCCTTTCC GGGACTTTCG CTTTCCCCCT CCCTATTGCC |
| 2581 | ACGGCGGAAC TCATCGCCGC CTGCCTTGCC CGCTGCTGGA CAGGGGCTCG GCTGTTGGGC |
| 2641 | ACTGACAATT CCGTGGTGTT GTCGGGGAAA TCATCGTCCT TTCCTTGGCT GCTCGCCTGT |
| 2701 | GTTGCCACCT GGATTCTGCG CGGGACGTCC TTCTGCTACG TCCCTTCGGC CCTCAATCCA |
| 2761 | GCGGACCTTC CTTCCCGCGG CCTGCTGCCG GCTCTGCGGC CTCTTCCGCG TCTTCGCCTT |
| 2821 | CGCCCTCAGA CGAGTCGGAT CTCCCTTTGG GCCGCCTCCC CGCATCGATA CCGTCGACCT |
| 2881 | CGAGGGAATT AATTCGAGCT CGGTACCTTT AAGACCAATG ACTTACAAGG CAGCTGTAGA |
| 2941 | TCTTAGCCAC TTTTTAAAAG AAAAGGGGGG ACTGGAAGGG CTAATTCACT CCCAACGAAG |
| 3001 | ACAAGATCTG CTTTTTGCTT GTACTGGGTC TCTCTGGTTA GACCAGATCT GAGCCTGGGA |
| 3061 | GCTCTCTGGC TAACTAGGGA ACCCACTGCT TAAGCCTCAA TAAAGCTTGC CTTGAGTGCT |
| 3121 | TCAAGTAGTG TGTGCCCGTC TGTTGTGTGA CTCTGGTAAC TAGAGATCCC TCAGACCCTT |
| 3181 | TTAGTCAGTG TGGAAAATCT CTAGCAGCAT CTAGAATTAA TTCCGTGTAT TCTATAGTGT |
| 3241 | CACCTAAATC GTATGTGTAT GATACATAAG GTTATGTATT AATTGTAGCC GCGTTCTAAC |
| 3301 | GACAATATGT ACAAGCCTAA TTGTGTAGCA TCTGGCTTAC TGAAGCAGAC CCTATCATCT |
| 3361 | CTCTCGTAAA CTGCCGTCAG AGTCGGTTTG GTTGGACGAA CCTTCTGAGT TTCTGGTAAC |
| 3421 | GCCGTCCCGC ACCCGGAAAT GGTCAGCGAA CCAATCAGCA GGGTCATCGC TAGCCAGATC |
| 3481 | CTCTACGCCG GACGCATCGT GGCCGGCATC ACCGGCGCCA CAGGTGCGGT TGCTGGCGCC |
| 3541 | TATATCGCCG ACATCACCGA TGGGGAAGAT CGGGCTCGCC ACTTCGGGCT CATGAGCGCT |
| 3601 | TGTTTCGGCG TGGGTATGGT GGCAGGCCCC GTGGCCGGGG GACTGTTGGG CGCCATCTCC |
| 3661 | TTGCATGCAC CATTCCTTGC GGCGGCGGTG CTCAACGGCC TCAACCTACT ACTGGGCTGC |
| 3721 | TTCCTAATGC AGGAGTCGCA TAAGGGAGAG CGTCGAATGG TGCACTCTCA GTACAATCTG |
| 3781 | CTCTGATGCC GCATAGTTAA GCCAGCCCCG ACACCCGCCA ACACCCGCTG ACGCGCCCTG |
| 3841 | ACGGGCTTGT CTGCTCCCGG CATCCGCTTA CAGACAAGCT GTGACCGTCT CCGGGAGCTG |
| 3901 | CATGTGTCAG AGGTTTTCAC CGTCATCACC GAAACGCGCG AGACGAAAGG GCCTCGTGAT |
| 3961 | ACGCCTATTT TTATAGGTTA ATGTCATGAT AATAATGGTT TCTTAGACGT CAGGTGGCAC |
| 4021 | TTTTCGGGGA AATGTGCGCG GAACCCCTAT TTGTTTATTT TTCTAAATAC ATTCAAATAT |
| 4081 | GTATCCGCTC ATGAGACAAT AACCCTGATA AATGCTTCAA TAATATTGAA AAAGGAAGAG |
| 4141 | TATGAGTATT CAACATTTCC GTGTCGCCCT TATTCCCTTT TTTGCGGCAT TTTGCCTTCC |
| 4201 | TGTTTTTGCT CACCCAGAAA CGCTGGTGAA AGTAAAAGAT GCTGAAGATC AGTTGGGTGC |
| 4261 | ACGAGTGGGT TACATCGAAC TGGATCTCAA CAGCGGTAAG ATCCTTGAGA GTTTTCGCCC |
| 4321 | CGAAGAACGT TTTCCAATGA TGAGCACTTT TAAAGTTCTG CTATGTGGCG CGGTATTATC |
| 4381 | CCGTATTGAC GCCGGGCAAG AGCAACTCGG TCGCCGCATA CACTATTCTC AGAATGACTT |
| 4441 | GGTTGAGTAC TCACCAGTCA CAGAAAAGCA TCTTACGGAT GGCATGACAG TAAGAGAATT |
| 4501 | ATGCAGTGCT GCCATAACCA TGAGTGATAA CACTGCGGCC AACTTACTTC TGACAACGAT |
| 4561 | CGGAGGACCG AAGGAGCTAA CCGCTTTTTT GCACAACATG GGGGATCATG TAACTCGCCT |
| 4621 | TGATCGTTGG GAACCGGAGC TGAATGAAGC CATACCAAAC GACGAGCGTG ACACCACGAT |
| 4681 | GCCTGTAGCA ATGGCAACAA CGTTGCGCAA ACTATTAACT GGCGAACTAC TTACTCTAGC |
| 4741 | TTCCCGGCAA CAATTAATAG ACTGGATGGA GGCGGATAAA GTTGCAGGAC CACTTCTGCG |
| 4801 | CTCGGCCCTT CCGGCTGGCT GGTTTATTGC TGATAAATCT GGAGCCGGTG AGCGTGGGTC |
| 4861 | TCGCGGTATC ATTGCAGCAC TGGGGCCAGA TGGTAAGCCC TCCCGTATCG TAGTTATCTA |
| 4921 | CACGACGGGG AGTCAGGCAA CTATGGATGA ACGAAATAGA CAGATCGCTG AGATAGGTGC |
| 4981 | CTCACTGATT AAGCATTGGT AACTGTCAGA CCAAGTTTAC TCATATATAC TTTAGATTGA |
| 5041 | TTTAAAACTT CATTTTTAAT TTAAAAGGAT CTAGGTGAAG ATCCTTTTTG ATAATCTCAT |
| 5101 | GACCAAAATC CCTTAACGTG AGTTTTCGTT CCACTGAGCG TCAGACCCCG TAGAAAAGAT |
| 5161 | CAAAGGATCT TCTTGAGATC CTTTTTTTCT GCGCGTAATC TGCTGCTTGC AAACAAAAAA |
| 5221 | ACCACCGCTA CCAGCGGTGG TTTGTTTGCC GGATCAAGAG CTACCAACTC TTTTTCCGAA |
| 5281 | GGTAACTGGC TTCAGCAGAG CGCAGATACC AAATACTGTT CTTCTAGTGT AGCCGTAGTT |
| 5341 | AGGCCACCAC TTCAAGAACT CTGTAGCACC GCCTACATAC CTCGCTCTGC TAATCCTGTT |
| 5401 | ACCAGTGGCT GCTGCCAGTG GCGATAAGTC GTGTCTTACC GGGTTGGACT CAAGACGATA |
| 5461 | GTTACCGGAT AAGGCGCAGC GGTCGGGCTG AACGGGGGGT TCGTGCACAC AGCCCAGCTT |
| 5521 | GGAGCGAACG ACCTACACCG AACTGAGATA CCTACAGCGT GAGCTATGAG AAAGCGCCAC |
| 5581 | GCTTCCCGAA GGGAGAAAGG CGGACAGGTA TCCGGTAAGC GGCAGGGTCG GAACAGGAGA |
| 5641 | GCGCACGAGG GAGCTTCCAG GGGGAAACGC CTGGTATCTT TATAGTCCTG TCGGGTTTCG |
| 5701 | CCACCTCTGA CTTGAGCGTC GATTTTTGTG ATGCTCGTCA GGGGGGCGGA GCCTATGGAA |
| 5761 | AAACGCCAGC AACGCGGCCT TTTTACGGTT CCTGGCCTTT TGCTGGCCTT TTGCTCACAT |
| 5821 | GTTCTTTCCT GCGTTATCCC CTGATTCTGT GGATAACCGT ATTACCGCCT TTGAGTGAGC |
| 5881 | TGATACCGCT CGCCGCAGCC GAACGACCGA GCGCAGCGAG TCAGTGAGCG AGGAAGCGGA |
| 5941 | AGAGCGCCCA ATACGCAAAC CGCCTCTCCC CGCGCGTTGG CCGATTCATT AATGCAGCTG |
| 6001 | TGGAATGTGT GTCAGTTAGG GTGTGGAAAG TCCCCAGGCT CCCCAGCAGG CAGAAGTATG |
| 6061 | CAAAGCATGC ATCTCAATTA GTCAGCAACC AGGTGTGGAA AGTCCCCAGG CTCCCCAGCA |
| 6121 | GGCAGAAGTA TGCAAAGCAT GCATCTCAAT TAGTCAGCAA CCATAGTCCC GCCCCTAACT |
| 6181 | CCGCCCATCC CGCCCCTAAC TCCGCCCAGT TCCGCCCATT CTCCGCCCCA TGGCTGACTA |
| 6241 | ATTTTTTTTA TTTATGCAGA GGCCGAGGCC GCCTCGGCCT CTGAGCTATT CCAGAAGTAG |
| 6301 | TGAGGAGGCT TTTTTGGAGG CCTAGGCTTT TGCAAAAAGC TTGGACACAA GACAGGCTTG |
| 6361 | CGAGATATGT TTGAGAATAC CACTTTATCC CGCGTCAGGG AGAGGCAGTG CGTAAAAAGA |
| 6421 | CGCGGACTCA TGTGAAATAC TGGTTTTTAG TGCGCCAGAT CTCTATAATC TCGCGCAACC |
| 6481 | TATTTTCCCC TCGAACACTT TTTAAGCCGT AGATAAACAG GCTGGGACAC TTCACATGAG |
| 6541 | CGAAAAATAC ATCGTCACCT GGGACATGTT GCAGATCCAT GCACGTAAAC TCGCAAGCCG |
| 6601 | ACTGATGCCT TCTGAACAAT GGAAAGGCAT TATTGCCGTA AGCCGTGGCG GTCTGTACCG |
| 6661 | GGTGCGTTAC TGGCGCGTGA ACTGGGTATT CGTCATGTCG ATACCGTTTG TATTTCCAGC |
| 6721 | TACGATCACG ACAACCAGCG CGAGCTTAAA GTGCTGAAAC GCGCAGAAGG CGATGGCGAA |
| 6781 | GGCTTCATCG TTATTGATGA CCTGGTGGAT ACCGGTGGTA CTGCGGTTGC GATTCGTGAA |
| 6841 | ATGTATCCAA AAGCGCACTT TGTCACCATC TTCGCAAAAC CGGCTGGTCG TCCGCTGGTT |
| 6901 | GATGACTATG TTGTTGATAT CCCGCAAGAT ACCTGGATTG AACAGCCGTG GGATATGGGC |
| 6961 | GTCGTATTCG TCCCGCCAAT CTCCGGTCGC TAATCTTTTC AACGCCTGGC ACTGCCGGGC |
| 7021 | GTTGTTCTTT TTAACTTCAG GCGGGTTACA ATAGTTTCCA GTAAGTATTC TGGAGGCTGC |
| 7081 | ATCCATGACA CAGGCAAACC TGAGCGAAAC CCTGTTCAAA CCCCGCTTTA AACATCCTGA |
| 7141 | AACCTCGACG CTAGTCCGCC GCTTTAATCA CGGCGCACAA CCGCCTGTGC AGTCGGCCCT |
| 7201 | TGATGGTAAA ACCATCCCTC ACTGGTATCG CATGATTAAC CGTCTGATGT GGATCTGGCG |
| 7261 | CGGCATTGAC CCACGCGAAA TCCTCGACGT CCAGGCACGT ATTGTGATGA GCGATGCCGA |
| 7321 | ACGTACCGAC GATGATTTAT ACGATACGGT GATTGGCTAC CGTGGCGGCA ACTGGATTTA |
| 7381 | TGAGTGGGCC CCGGATCTTT GTGAAGGAAC CTTACTTCTG TGGTGTGACA TAATTGGACA |
| 7441 | AACTACCTAC AGAGATTTAA AGCTCTAAGG TAAATATAAA ATTTTTAAGT GTATAATGTG |
| 7501 | TTAAACTACT GATTCTAATT GTTTGTGTAT TTTAGATTCC AACCTATGGA ACTGATGAAT |
| 7561 | GGGAGCAGTG GTGGAATGCC TTTAATGAGG AAAACCTGTT TTGCTCAGAA GAAATGCCAT |
| 7621 | CTAGTGATGA TGAGGCTACT GCTGACTCTC AACATTCTAC TCCTCCAAAA AAGAAGAGAA |
| 7681 | AGGTAGAAGA CCCCAAGGAC TTTCCTTCAG AATTGCTAAG TTTTTTGAGT CATGCTGTGT |
| 7741 | TTAGTAATAG AACTCTTGCT TGCTTTGCTA TTTACACCAC AAAGGAAAAA GCTGCACTGC |
| 7801 | TATACAAGAA AATTATGGAA AAATATTCTG TAACCTTTAT AAGTAGGCAT AACAGTTATA |
| 7861 | ATCATAACAT ACTGTTTTTT CTTACTCCAC ACAGGCATAG AGTGTCTGCT ATTAATAACT |
| 7921 | ATGCTCAAAA ATTGTGTACC TTTAGCTTTT TAATTTGTAA AGGGGTTAAT AAGGAATATT |
| 7981 | TGATGTATAG TGCCTTGACT AGAGATCATA ATCAGCCATA CCACATTTGT AGAGGTTTTA |
| 8041 | CTTGCTTTAA AAAACCTCCC ACACCTCCCC CTGAACCTGA AACATAAAAT GAATGCAATT |
| 8101 | GTTGTTGTTA ACTTGTTTAT TGCAGCTTAT AATGGTTACA AATAAAGCAA TAGCATCACA |
| 8161 | AATTTCACAA ATAAAGCATT TTTTTCACTG CATTCTAGTT GTGGTTTGTC CAAACTCATC |
| 8221 | AATGTATCTT ATCATGTCTG GATCAACTGG ATAACTCAAG CTAACCAAAA TCATCCCAAA |
| 8281 | CTTCCCACCC CATACCCTAT TACCACTGCC AATTACCTGT GGTTTCATTT ACTCTAAACC |
| 8341 | TGTGATTCCT CTGAATTATT TTCATTTTAA AGAAATTGTA TTTGTTAAAT ATGTACTACA |
| 8401 | AACTTAGTAG TTGGAAGGGC TAATTCACTC CCAAAGAAGA CAAGATATCC TTGATCTGTG |
| 8461 | GATCTACCAC ACACAAGGCT ACTTCCCTGA TTAGCAGAAC TACACACCAG GGCCAGGGGT |
| 8521 | CAGATATCCA CTGACCTTTG GATGGTGCTA CAAGCTAGTA CCAGTTGAGC CAGATAAGGT |
| 8581 | AGAAGAGGCC AATAAAGGAG AGAACACCAG CTTGTTACAC CCTGTGAGCC TGCATGGGAT |
| 8641 | GGATGACCCG GAGAGAGAAG TGTTAGAGTG GAGGTTTGAC AGCCGCCTAG CATTTCATCA |
| 8701 | CGTGGCCCGA GAGCTGCATC CGGAGTACTT CAAGAACTGC TGATATCGAG CTTGCTACAA |
| 8761 | GGGACTTTCC GCTGGGGACT TTCCAGGGAG GCGTGGCCTG GGCGGGACTG GGGAGTGGCG |
| 8821 | AGCCCTCAGA TCCTGCATAT AAGCAGCTGC TTTTTGCCTG TACTGGGTCT CTCTGGTTAG |
| 8881 | ACCAGATCTG AGCCTGGGAG CTCTCTGGCT AACTAGGGAA CCCACTGCTT AAGCCTCAAT |
| 8941 | AAAGCTTGCC TTGAGTGCTT CAAGTAGTGT GTGCCCGTCT GTTGTGTGAC TCTGGTAACT |
| 9001 | AGAGATCCCT CAGACCCTTT TAGTCAGTGT GGAAAATCTC TAGCAGTGGC GCCCGAACAG |
| 9061 | GGACTTGAAA GCGAAAGGGA AACCAGAGGA GCTCTCTCGA CGCAGGACTC GGCTTGCTGA |
| 9121 | AGCGCGCACG GCAAGAGGCG AGGGGCGGCG ACTGGTGAGT ACGCCAAAAA TTTTGACTAG |
| 9181 | CGGAGGCTAG AAGGAGAGAG ATGGGTGCGA GAGCGTCAGT ATTAAGCGGG GGAGAATTAG |
| 9241 | ATCGCGATGG GAAAAAATTC GGTTAAGGCC AGGGGGAAAG AAAAAATATA AATTAAAACA |
| 9301 | TATAGTATGG GCAAGCAGGG AGCTAGAACG ATTCGCAGTT AATCCTGGCC TGTTAGAAAC |
| 9361 | ATCAGAAGGC TGTAGACAAA TACTGGGACA GCTACAACCA TCCCTTCAGA CAGGATCAGA |
| 9421 | AGAACTTAGA TCATTATATA ATACAGTAGC AACCCTCTAT TGTGTGCATC AAAGGATAGA |
| 9481 | GATAAAAGAC ACCAAGGAAG CTTTAGACAA GATAGAGGAA GAGCAAAACA AAAGTAAGAC |
| 9541 | CACCGCACAG CAAGCGGCCG GCCGCTGATC TTCAGACCTG GAGGAGGAGA TATGAGGGAC |
| 9601 | AATTGGAGAA GTGAATTATA TAAATATAAA GTAGTAAAAA TTGAACCATT AGGAGTAGCA |
| 9661 | CCCACCAAGG CAAAGAGAAG AGTGGTGCAG AGAGAAAAAA GAGCAGTGGG AATAGGAGCT |
| 9721 | TTGTTCCTTG GGTTCTTGGG AGCAGCAGGA AGCACTATGG GCGCAGCGTC AATGACGCTG |
| 9781 | ACGGTACAGG CCAGACAATT ATTGTCTGGT ATAGTGCAGC AGCAGAACAA TTTGCTGAGG |
| 9841 | GCTATTGAGG CGCAACAGCA TCTGTTGCAA CTCACAGTCT GGGGCATCAA GCAGCTCCAG |
| 9901 | GCAAGAATCC TGGCTGTGGA AAGATACCTA AAGGATCAAC AGCTCCTGGG GATTTGGGGT |
| 9961 | TGCTCTGGAA AACTCATTTG CACCACTGCT GTGCCTTGGA ATGCTAGTTG GAGTAATAAA |
| 10021 | TCTCTGGAAC AGATTTGGAA TCACACGACC TGGATGGAGT GGGACAGAGA AATTAACAAT |
| 10081 | TACACAAGCT TAATACACTC CTTAATTGAA GAATCGCAAA ACCAGCAAGA AAAGAATGAA |
| 10141 | CAAGAATTAT TGGAATTAGA TAAATGGGCA AGTTTGTGGA ATTGGTTTAA CATAACAAAT |
| 10201 | TGGCTGTGGT ATATAAAATT ATTCATAATG ATAGTAGGAG GCTTGGTAGG TTTAAGAATA |
| 10261 | GTTTTTGCTG TACTTTCTAT AGTGAATAGA GTTAGGCAGG GATATTCACC ATTATCGTTT |
| 10321 | CAGACCCACC TCCCAACCCC GAGGGGACCC GACAGGCCCG AAGGAATAGA AGAAGAAGGT |
| 10381 | GGAGAGAGAG ACAGAGACAG ATCCATTCGA TTAGTGAACG GATCTCGACG GTATCGCCAA |
| 10441 | ATGGCAGTAT TCATCCACAA TTTTAAAAGA AAAGGGGGGA TTGGGGGGTA CAGTGCAGGG |
| 10501 | GAAAGAATAG TAGACATAAT AGCAACAGAC ATACAAACTA AAGAATTACA AAAACAAATT |
| 10561 | ACAAAAATTC AAAATTTTCG GGTTTATTAC AGGGACAGCA GAGATCCAGT TTGGATCGAT |
| 10621 | AAGCTTGATA TCGAATTCCT GCAGCCCCGA TAAAATAAAA GATTTTATTT AGTCTCCAGA |
| 10681 | AAAAGGGGGG AATGAAAGAC CCCACCTGTA GGTTTGGCAA GCTAGCTGCA GTAACGCCAT |
| 10741 | TTTGCAAGGC ATGGAAAAAT ACCAAACCAA GAATAGAGAA GTTCAGATCA AGGGCGGGTA |
| 10801 | CATGAAAATA GCTAACGTTG GGCCAAACAG GATATCTGCG GTGAGCAGTT TCGGCCCCGG |
| 10861 | CCCGGGGCCA AGAACAGATG GTCACCGCAG TTTCGGCCCC GGCCCGAGGC CAAGAACAGA |
| 10921 | TGGTCCCCAG ATATGGCCCA ACCCTCAGCA GTTTCTTAAG ACCCATCAGA TGTTTCCAGG |
| 10981 | CTCCCCCAAG GACCTGAAAT GACCCTGCGC CTTATTTGAA TTAACCAATC AGCCTGCTTC |
| 11041 | TCGCTTCTGT TCGCGCGCTT CTGCTTCCCG AGCTCTATAA AAGAGCTCAC AACCCCTCAC |
| 11101 | TCGGCGCGCC AGTCCTCCGA CAGACTGAGT CGCCCGGGGG GGATCTGGAG CTCTCGAGAA |
| 11161 | TTCTCACGCG TCAAGTGGAG CAAGGCAGGT GGACAGTGAT GGCCTTACCA GTGACCGCCT |
| 11221 | TGCTCCTGCC GCTGGCCTTG CTGCTCCACG CCGCCAGGCC GGAGCAGAAG CTGATCAGCG |
| 11281 | AGGAGGACCT GGAGGAGGAC CTG |

//
(SEQ ID NO: 29)

1. A chimeric antigen receptor (CAR) polypeptide comprising a TM4SF1 antigen binding domain, a transmembrane domain, and an intracellular signaling domain.

2. The CAR polypeptide of claim 1, wherein the TM4SF1 antigen binding domain is an antibody fragment or an antigen-binding fragment that specifically binds to TM4SF1.

3. The CAR polypeptide of claim 1, wherein the TM4SF1 antigen binding domain is a Fab or a single-chain variable fragment (scFv) of an antibody that specifically binds TM4SF1.

4. The CAR polypeptide of claim 1, wherein the TM4SF1 antigen binding domain comprises the amino acid sequence of
(SEQ ID NO: 86)
EVILVESGGGLVKPGGSLKLSCAASGFTFSSFAMSWVRQTPEKRLEWVA
TISSGSIYIYYTDGVKGRFTISRDNAKNTVHLQMSSLRSEDTAMYYCAR
RGIYYGYDGYAMDYWGQGTSVTVSGGGGSGGGGSGGGGSAVVMTQTPLS
LPVSLGDQASISCRSSQSLVHSNGNTYLHWYMQKPGQSPKVLIYKVSNR
FSGVPDRFSGSGSGTDFTLKISRVEADDLGIYFCSQSTHIPLAFGAGTK
LELK
or
(SEQ ID NO: 87)
AVVMTQTPLSLPVSLGDQASISCRSSQSLVHSNGNTYLHWYMQKPGQSP
KVLIYKVSNRFSGVPDRFSGSGSGTDFTLKISRVEADDLGIYFCSQSTH
IPLAFGAGTKLELKGGGGSGGGGSGGGGSEVILVESGGGLVKPGGSLKL
SCAASGFTFSSFAMSWVRQTPEKRLEWVATISSGSIYIYYTDGVKGRFT
ISRDNAKNTVHLQMSSLRSEDTAM.

5. The CAR polypeptide of claim 1, wherein the TM4SF1 binding domain comprises a heavy chain variable domain comprising a CDR3 domain comprising an amino acid sequence that has at least 75% identity to SEQ ID NO: 1, 2, 3, 4, 5, 6, 7, 8, 9, 10, 11, or 12; a CDR2 domain comprising an amino acid sequence that has at least 75% identity to SEQ ID NO: 13, 14, 15, 16, 17, 18, 19, 20, 21, 22; and a CDR1 domain comprising an amino acid sequence that has at least 75% identity to SEQ ID NO: 23, 24, 25, 26, 27, 28, 29, 30, 31; and
a light chain variable domain comprising a CDR3 domain comprising an amino acid sequence that has at least 75% identity to SEQ ID NO: 32, 33, 34, 35, 36, 37, 38, 39, 40;
a CDR2 domain comprising an amino acid sequence that has at least 75% identity to SEQ ID NO: 41, 42, 43, 44, 45, 46, 47, 48, 49; and a CDR1 comprising an amino acid sequence that has at least 75% identity to SEQ ID NO: 50, 51, 52, 53, 54, 55, 56, 57, 58, 59, 60, 61, 62.

6. The CAR polypeptide of claim 1, wherein the intracellular signaling domain further comprises a co-stimulatory signaling region.

7. The CAR polypeptide of claim 1, wherein the co-stimulatory signaling region comprises the cytoplasmic domain of a costimulatory molecule selected from the group consisting of 4-1BB, CD28, CD27, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

8. The CAR polypeptide of claim 1, wherein the intracellular signaling domain is a T cell signaling domain.

9. The CAR polypeptide of claim 1, wherein the intracellular signaling domain comprises a CD3 zeta (CD3ζ) signaling domain.

10. The CAR polypeptide of claim 1, wherein the intracellular signaling domain comprises a CD3ζ signaling domain and a co-stimulatory signaling region, wherein the co-stimulatory signaling region comprises the cytoplasmic domain of CD28 or 4-1BB.

11. The CAR polypeptide of claim 1, wherein the transmembrane domain comprises a transmembrane domain of a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain.

12. The CAR polypeptide of claim 1, wherein the transmembrane domain is located between the TM4SF1 antigen binding domain and the intracellular signaling domain.

13. The CAR polypeptide of claim 1, further comprising a tag.

14. (canceled)

15. (canceled)

16. The CAR polypeptide of claim 1 further comprising a hinge region.

17. (canceled)

18. (canceled)

19. The CAR polypeptide of claim 1, wherein the CAR polypeptide comprises:
a TM4SF1 antigen binding domain comprising
a heavy chain variable domain comprising a CDR3 domain comprising an amino acid sequence that has at least 75% identity to SEQ ID NO: 1, 2, 3, 4, 5, 6, 7, 8, 9, 10, 11, or 12; a CDR2 domain comprising an amino acid sequence that has at least 75% identity to SEQ ID NO: 13, 14, 15, 16, 17, 18, 19, 20, 21, 22; and a CDR1 domain comprising an amino acid sequence that has at least 75% identity to SEQ ID NO: 23, 24, 25, 26, 27, 28, 29, 30, 31; and
a light chain variable domain comprising a CDR3 domain comprising an amino acid sequence that has at least 75% identity to SEQ ID NO: 32, 33, 34, 35, 36, 37, 38, 39, 40; a CDR2 domain comprising an amino acid sequence that has at least 75% identity to SEQ ID NO: 41, 42, 43, 44, 45, 46, 47, 48, 49; and a CDR1 comprising an amino acid sequence that has at least 75% identity to SEQ ID NO: 50, 51, 52, 53, 54, 55, 56, 57, 58, 59, 60, 61, 62;
an IgG4 spacer,
a CD8 hinge,
a CD8 transmembrane domain,
a 4-1BB costimulatory domain; and
a CD3ζ chain,

20. A nucleic acid sequence capable of encoding the CAR polypeptide of claim 1.

21. A vector comprising the nucleic acid sequence of claim 20.

22.-28. (canceled)

23. (canceled)

29. A T cell expressing the CAR polypeptide of claim 1.

30. (canceled)

31. (canceled)

32. A method of treating a subject having a cancer associated with increased TM4SF1 expression comprising administering an effective amount of a composition comprising a T cell genetically modified to express the CAR polypeptide of claim 1 to a subject in need thereof.

33.-36. (canceled)

37. A method of killing TM4SF1 positive cells comprising administering an effective amount of a T cell genetically modified to express the CAR polypeptide of claim 1 to a sample comprising TM4SF1 positive cells.

38. (canceled)
